(19) United States
(12) Patent Application Publication
(10) Pub. No.: US 2024/0132880 A1
(43) Pub. Date: Apr. 25, 2024

(54) TRANSCRIPTIONAL PROGRAMMING IN A BACTEROIDES CONSORTIUM

(71) Applicant: Georgia Tech Research Corporation, Atlanta, GA (US)

(72) Inventor: Corey J. Wilson, Atlanta, GA (US)

(21) Appl. No.: 18/490,880

(22) Filed: Oct. 19, 2023

Related U.S. Application Data
(60) Provisional application No. 63/380,434, filed on Oct. 21, 2022.

(51) Int. Cl. C12N 15/11; C12N 9/22; C12N 15/10

(52) U.S. Cl. CPC C12N 15/11; C12N 9/22; C12N 15/102; C12N 2310/20

(57) ABSTRACT

The present disclosure provides nucleic acid constructs and cell compositions for transcriptionally modifying a bacterial population within the gastrointestinal tract of a subject, and methods of use thereof.

### CROSS-REFERENCE TO RELATED APPLICATIONS

This application claims the benefit of U.S. Provisional Application No. 63/380,434, filed on Oct. 21, 2022, which is expressly incorporated herein by reference in their entirety.

### STATEMENT REGARDING FEDERALLY SPONSORED RESEARCH

This invention was made with government support under Grant No. 1934836 awarded by the National Science Foundation. The government has certain rights in the invention.

### REFERENCE TO SEQUENCE LISTING

A Sequence Listing conforming to the rules of WIPO Standard ST.26 is hereby incorporated by reference. Said Sequence Listing has been filed as an electronic document via Patent Center in ASCII format encoded as XML. The electronic document, created on Oct. 17, 2023, is entitled “10034-187US1_ST26.xml”, and is 583,550 bytes in size.

### FIELD

The present disclosure relates to nucleic acid constructs and uses thereof.

### BACKGROUND

The human gastrointestinal (GI) tract harbors a microbial ecosystem of enormous complexity that contributes significantly to the health of the host. Evidence continues to emerge connecting the GI microbiota with health and disease states not only in the immediate vicinity of the GI tract, but systemically as well. Many studies involving the GI microbiota leverage metagenomic data to investigate how its highly variable composition across age and demographics can be connected to health conditions. In contrast, several studies have investigated the impact of individual species on the microbiota through functional genomics and targeted manipulation of GI communities. As the understanding of the gut microbiota expands in scope and depth, it is conceivable to engineer intelligent microbial consortia that reside in the human body. However, the vast majority of microbes inhabiting the GI tract are obligate anaerobes that are not readily amenable to genetic manipulation. This poses a challenge to synthetic biologists who seek to reprogram these microbes to perform useful functions beyond native capabilities. Therefore, what is needed are compositions and cells for programming GI microbiota.

### SUMMARY

The present disclosure provides constructs and cell compositions for reprogramming a GI microbiome. The present disclosure also provides methods using constructs and cell compositions to modify and/or monitor a GI microbiome.

In one aspect, disclosed herein is a construct comprising a plurality of nucleic acid sequences encoding a first group of one or more regulatory core domains, a second group of one or more regulatory core domains, one or more DNA binding domains, wherein the first group of the one or more regulatory core domains and the second group of the one or more regulatory core domains are each linked to one of the DNA binding domains, and one or more DNA operator elements, wherein the one or more DNA operator elements are each specifically recognized by one of the DNA binding domains.

In some embodiments, the construct further comprises SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or variants thereof.

Ins some embodiments, the plurality of nucleic acid sequences comprises any combination of SEQ ID NO: 1, SEQ ID NO: 2, SEQ ID NO: 3, SEQ ID NO: 4, SEQ ID NO: 5, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or variants thereof.

In some embodiments, the first group of one or more regulatory core domains comprises at least one repressor or at least one anti-repressor, or a combination thereof. In some embodiments, the second group of one or more regulatory core domains comprise at least one repressors, at least one anti-repressors, or a combination thereof.

In some embodiments, the first group of one or more regulatory core domains are specifically recognized by a first agent. In some embodiments, the first agent is isopropyl-β-D-1-thiogalactopyranoside.

In some embodiments, the second group of one or more regulatory core domains are specifically recognized by a second agent. In some embodiments, the second agent is D-ribose.

In some embodiments, the first and second group of the one or more regulatory core domains are linked to a same DNA binding domain. In some embodiments, the first and second group of the one or more regulatory core domains are linked to different DNA binding domains.

In some embodiments, the construct comprises a plurality of nucleic acid sequences encoding a first group of two regulatory core domains, a second group of two regulatory core domains, three DNA binding domains, wherein the first group of the regulatory core domains and second group of the regulatory core domains are each linked to one of the three DNA binding domains, and three DNA operator elements that are each specifically recognized by one of the three DNA binding domains.

In some embodiments, the construct further comprises a nucleic acid sequence encoding a reporter. In some embodiments, the construct further comprises a nucleic acid sequence encoding a dead Cas9 endonuclease (dCas9) and a single guide RNA (sgRNA).

In some embodiments, the nucleic acid sequence encodes any combination of SEQ ID NO: 6, SEQ ID NO: 16, SEQ ID NO: 17, SEQ ID NO: 18, SEQ ID NO: 19, or variants thereof. In some embodiments, the construct further comprises SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, or variants thereof.

In one aspect, disclosed herein is a cell comprising a construct comprising a plurality of nucleic acid sequences encoding a first group of one or more regulatory core domains, a second group of one or more regulatory core domains, one or more DNA binding domains, wherein the first group of the one or more regulatory core domains and the second group of the one or more regulatory core domains are each linked to one of the DNA binding domains, and one or more DNA operator elements, wherein the one or more DNA operator elements are each specifically recognized by one of the DNA binding domains.

In some embodiments, the cell comprises the construct of any preceding aspect.

In some embodiments, the cell is a bacterial cell. In some embodiments, the bacterial cell is a bacterium of gastrointestinal tract microbiota. In some embodiments, the bacterial cell is a species of Bacteroides genus selected from B. thetaiotaomicron (Bt), B. fragilis (Bf), B. vulgatus (Bv), B. ovatus (Bo), or B. uniformis (Bu).

In one aspect, disclosed herein is a method of modifying a gastrointestinal tract microbiome in a subject, comprising administering to the subject an effective amount of a cell comprising a construct comprising a plurality of nucleic acid sequences encoding a first group of one or more regulatory core domains, a second group of one or more regulatory core domains, one or more DNA binding domains, wherein the first group of the one or more regulatory core domains and the second group of the one or more regulatory core domains are each linked to one of the DNA binding domains, and one or more DNA operator elements, wherein the one or more DNA operator elements are each specifically recognized by one of the DNA binding domains.

In some embodiments, the method of modifying a gastrointestinal tract microbiome in a subject, comprising administering to the subject an effective amount of the cell of any preceding aspect, wherein the cell comprises the construct of any preceding aspect.

In one aspect, disclosed herein is a method of monitoring a gastrointestinal tract microbiome in a subject, comprising administering to the subject an effective amount of a cell comprising a construct comprising a plurality of nucleic acid sequences encoding a first group of one or more regulatory core domains, a second group of one or more regulatory core domains, one or more DNA binding domains, wherein the first group of the one or more regulatory core domains and the second group of the one or more regulatory core domains are each linked to one of the DNA binding domains, and one or more DNA operator elements, wherein the one or more DNA operator elements are each specifically recognized by one of the DNA binding domains.

In some embodiments, the method of monitoring a gastrointestinal tract microbiome in a subject, comprising administering to the subject an effective amount of the cell of any preceding aspect, wherein the cell comprises the construct of any preceding aspect.

### BRIEF DESCRIPTION OF FIGURES

The accompanying figures, which are incorporated in and constitute a part of this specification, illustrate several aspects described below.

FIGS. 1A, 1B, 1C, 1D, and 1E show the regulatory performance of transcription factors in Bacteroides species. FIG. 1A shows the initial set of transcription factors (TFs) tested in B. thetaiotaomicron (left box). The four signal-distinct regulatory core domains (RCDs) are shown with their repressor and anti-repressor cartoons. The cognate ligand for each RCD is shown as a colored hexagon. Each RCD can be paired with one of seven DNA-binding domains (DBDs) that each recognize and bind to a unique cognate DNA operator. Each DBD is abbreviated with a three-letter code where the three letters correspond to the residues located at positions 17, 18, and 22 of the LacI DBD. Cognate DBD-operator interactions are shown as the same color. The reduced set of functional TFs in B. thetaiotaomicron is shown in the middle box. The repressor and anti-repressor phenotypes are illustrated in the right boxes. FIG. 1B shows the dynamic range of LacI (I⁺) TFs with alternate DNA recognition (ADR) when paired with cognate operators. Regulated promoters are illustrated at the bottom of the figure. Each box corresponds to the inducible promoter shown in the left columns when deployed in the Bacteroides species labeled below (B. thetaiotaomicron (Bt), B. fragilis (Bf), B. vulgatus (Bv), B. ovatus (Bo), B. uniformis (Bu)). The dynamic range of each promoter is presented next to its corresponding box which is shaded according to the legends at the top of each panel. Strains harboring inducible promoters were grown in the absence and presence of 10 mM inducer and assayed for luciferase activity (Methods). Dynamic range is presented as the high output state divided by the low output state. FIG. 1C shows the dynamic range of RbsR (R⁺) transcription factors when paired with cognate operators. FIG. 1D shows the dynamic range of anti-LacI (I^{A}) transcription factors when paired with cognate operators. FIG. 1E show the dynamic range of anti-RbsR (R^{A}) transcription factors when paired with cognate operators. See Supplementary FIGS. 1-2 for extended data. Source data are provided as a Source Data file. Data represent the average of n=6 biological replicates.

FIGS. 2A, 2B, 2C, and 2D show the single-promoter logic gates constructed in B. thetaiotaomicron. FIG. 2A shows the AND gate is constructed by directing two repressors to the same operator. We refer to the genetic architecture of directing two or more TFs to a single operator as series-parallel (SE-PA). Bar charts show luciferase activity presented as luminescence per colony forming unit (CFU) (Methods). Each bar corresponds to the ligand condition shown to its left (empty, no ligand; grey, IPTG; purple, D-ribose; grey and purple, IPTG and D-ribose). Strains harboring logic gates were grown in the presence of all combinations of both inducers (each at a final concentration of 10 mM) and assayed for luciferase activity (Methods). Adjacent to each bar chart, the fold-change is presented (blue for repression and purple for anti-repression). FIG. 2B shows the NOR gate constructed by directing two anti-repressors to the same operator. FIG. 2C shows the A NIMPLY B gate is constructed by directing an I⁺ and a R^{A} to the same promoter. FIG. 2D shows the B NIMPLY A gate is constructed by directing an I^{A} and a R⁺ to the same promoter. Source data are provided as a Source Data file. Data represent the average of n=6 biological replicates. Error bars correspond to the SEM of these measurements.

FIGS. 3A, 3B, 3C, 3D, 3E, 3F, and 3G show the logic gates and circuit compression in five Bacteroides species. FIG. 3A shows the degree of AND gate circuit compression compared to published circuits (left) along with AND gate performance in five Bacteroides species (right). Degree of circuit compression is represented by the number of regulated promoters required to construct the logic gate (TP, transcriptional programming; CP, Cello programming; WP, multicellular wires programming; NP, Boolean NOR programming). Note, the gray and (*) bars in the circuit compression summaries indicate an apparent or ad hoc gate. Strains harboring circuits were grown in the presence of all combinations of both inducers (each at a final concentration of 10 mM) and assayed for luciferase activity (Methods). FIG. 3B shows the NOR gate compression and performance in five Bacteroides. FIG. 3C shows the OR gate compression and performance in five Bacteroides (see Supplementary FIG. 15G for corresponding genetic architecture). FIG. 3D shows the NAND gate compression and performance in five Bacteroides (see Supplementary FIG. 15H for corresponding genetic architecture). FIG. 3E shows the XNOR gate compression and performance in five Bacteroides (see Supplementary FIG. 15I for corresponding genetic architecture). FIG. 3F shows the XOR gate compression and performance in five Bacteroides. FIG. 3G shows the wiring diagram for XOR construction using transcriptional programming (top) compared to Bt Cello programming (bottom). Regulated promoters used for each logic gate are shown in the bottom right corner of each left-hand box (also see FIG. 1 for more detail). Source data are provided as a Source Data file. Data represent the average of n=6 biological replicates. Error bars correspond to the SEM of these measurements.

FIGS. 4A, 4B, 4C, 4D, 4E, and 4F show the coupling transcriptional programming with CRISPR interference. FIG. 4A shows the wiring diagram for NanoLuc knockdown based on a BUFFER gate controlling a sgRNA (top). I⁺_{YQR} was used to regulate a sgRNA targeting the NanoLuc gene. Strains harboring the circuit were grown in the absence and presence of 10 mM IPTG and assayed for luciferase activity (bottom) (Methods). Dynamic range is illustrated with shading according to the legend below the graph (purple representing anti-induction, blue representing induction) and is calculated as described previously. FIG. 4B shows the wiring diagram for NanoLuc knockdown based on a NOT gate controlling a sgRNA (top). I^{A}_{YQR} was used to regulate a sgRNA targeting the NanoLuc gene. Strains harboring the circuit were assayed as in FIG. 4A to determine performance (bottom). FIG. 4C shows the wiring diagram of CRISPRi circuit targeting endogenous SusC-like genes. Strains were created with X⁺_{YQR} (X=I or R) regulating a sgRNA specific to the SusC homologue of interest. FIG. 4D shows the cartoon of PUL organization, highlighting the function of the SusC-like importer. Colors of proteins and complexes correspond to the genes shown in c and are listed in the legend at the top right corner. FIG. 4E shows the growth curves of B. ovatus (left) and B. uniformis (right) harboring circuits shown in c with I⁺_{YQR} as the sgRNA regulator. Strains harboring CRISPRi circuits were grown in the absence and presence of 10 mM IPTG in minimal media containing inulin as the only carbon source (Methods). FIG. 4F is similar to FIG. 4E, but shows the sgRNA regulator is now R⁺_{YQR}. Strains harboring these circuits were grown in the absence and presence of 1 mM D-ribose in inulin minimal media. Source data are provided as a Source Data file. For luciferase assays, data represent the average of n=6 biological replicates. For OD600 growth curves, data represent the average of n=3 biological replicates. Error bars correspond to the SEM of these measurements.

FIGS. 5A, 5B, 5C, 5D, and 5E show the advanced regulation of endogenous carbon utilization genes. FIG. 5A shows the wiring diagram for an AND gate controlling a sgRNA output targeted to an endogenous gene (left). The sgRNA is the same as in FIG. 4E. Growth curves are shown for B. uniformis harboring this circuit when grown in inulin minimal media in the presence of all combinations of both inducers (10 mM IPTG and 1 mM D-ribose) (right). FIG. 5B is similar to 5A, but shows a NOR gate controlling a sgRNA targeted to the B. ovatus SusC-like gene involved in inulin import. FIG. 5C is similar to 5B, but shows an OR gate controlling the sgRNA output. Source data are provided as a Source Data file. For OD600 growth curves, data represent the average of n=3 biological replicates. Error bars correspond to the SEM of these measurements.

FIGS. 6A, 6B, 6C, 6D, and 6E show the controlling community composition in co-culture. FIG. 6A shows the demonstration of asymmetric programming of two species in co-culture. The B. uniformis strain described in FIG. 5A was co-cultured with the B. ovatus strain described in FIG. 5C in inulin minimal media. Each strain was intentionally constructed to harbor a different antibiotic resistance so that they could be differentiated when plated on BHI agar with appropriate antibiotics. The left bar chart shows the density of the B. uniformis strain over time as calculated by dilution plating (Methods). The right bar chart shows the density of B. ovatus over time. Four co-cultures were grown in parallel, each with a different combination of inducers. White bars represent medium with no ligand, grey bars represent medium with 10 mM IPTG, purple bars represent medium with 1 mM D-ribose, and striped bars represent medium with both inducers. FIG. 6B shows a cartoon illustrating the co-culture composition with no ligand present in the medium. FIG. 6C shows a cartoon illustrating the co-culture composition with IPTG present in the medium. FIG. 6D shows a cartoon illustrating the co-culture composition with D-ribose present in the medium. FIG. 6E shows a cartoon illustrating the co-culture composition with both IPTG and D-ribose present in the medium. Source data are provided as a Source Data file. Data represent the average of n=3 technical replicates (see FIG. 16 for additional data and information). Error bars correspond to the SEM of these measurements.

FIGS. 7A, 7B, 7C, 7D, 7E, and 7F show the sequential programming in co-culture. FIG. 7A shows a co-culture composed of B. thetaiotaomicron and B. ovatus harboring different logic gates. B. thetaiotaomicron integrated with an AND gate was grown in the same TYG culture as B. ovatus integrated with a NOR gate (both gates regulating luciferase expression). Co-cultures were grown in the presence of all combinations of inducers (each at a final concentration of 10 mM) yielding four total cultures which were independently assayed for luciferase activity (Methods). FIG. 7B shows a sequential programs derived from the “No Ligand” culture in FIG. 7A. The initial co-culture was used to seed fresh media containing additional inducers (i.e., IPTG, ribose, or both ligands). These new co-cultures were grown and assayed for luciferase activity (FIG. 7F, left bar chart). FIG. 7C shows the sequential program derived from the “+IPTG” culture in FIG. 7A. The initial co-culture was used to seed fresh medium containing both ligands. This new co-culture was subsequently assayed for luciferase activity (FIG. 7F, middle bar chart). FIG. 7D shows the sequential program derived from the “+Ribose” culture in FIG. 7A. The initial co-culture was used to seed fresh medium containing both ligands. This new co-culture was subsequently assayed for luciferase activity (FIG. 7F, right bar chart). FIG. 7E shows the luciferase activity of co-cultures on day 1. Numbered circles correspond to the co-cultures illustrated in FIG. 7A. FIG. 7F shows the luciferase activity of co-cultures on day 2. Numbered circles correspond to the co-cultures illustrated in FIGS. 7B, 7C, and 7D. Source data are provided as a Source Data file. Data represent the average of n=3 biological replicates. Error bars correspond to the SEM of these measurements.

FIGS. 8A and 8B show the components of modular transcription factors used for transcriptional programming. FIG. 8A shows the summary of transcription factor architecture and nomenclature used in this study. Each transcription factor is composed of a regulatory core domain (RCD) that binds to a unique small molecule ligand and a DNA-binding domain (DBD) that binds to a specific operator (op). The cognate ligand for each RCD is shown as a colored hexagon. The repressor and anti-repressor phenotypes are illustrated in the middle. Transcription factors utilize alternate DNA recognition (ADR) which is comprised of 5 DBDs that recognize 5 unique operators which are color-coded. Each DBD is abbreviated with a three-letter code where the three letters correspond to the residues located at positions 17, 18, and 22 of the LacI DBD. Each operator is abbreviated with a three-letter code that corresponds to the critically-recognized bases of the synthetic DNA sequence. FIG. 8B shows the full panel of 20 transcription factors used. All 20 transcription factors are illustrated with corresponding abbreviations used.

FIGS. 9A and 9B show the regulatory performance of transcription factors in Bacteroides species. Extended data related to FIG. 1. Low and high states for every cognate TF-promoter pair are shown for the five Bacteroides species. Bar pairs correspond to the squares in FIG. 1 with the dynamic range being the ratio of high and low states. To direct each transcription factor, in-tandem operator-promoters composed of two DNA operators were used, one intercalated between the −33 and −7 hexamer and the other proximal to the TSS, also see Example 2. Each set of transcription factors for a given logical operation could be independently directed to five separate cognate operator-promoters—i.e., PO1, Ptta, Pttg, Pagg, or Pgac—without cross interaction (also see FIG. 10). Data represent the average of n=6 biological replicates. Error bars correspond to the SEM of these measurements. Induction of each promoter was determined to be statistically significant (P<0.001) using Welch's two-tailed unequal variances t-test. Exact P-values can be found in the Source Data file.

FIGS. 10A, 10B, 10C, 10D, 10E, and 10F show the demonstration of orthogonality between DNA-binding domains and synthetic operators. FIG. 10A shows the PO1 orthogonality test. The 16 non-cognate TFs were tested for their ability to regulate PO1. Separate strains were created harboring the inducible promoter and each of the 16 non-cognate TFs. These strains were grown in the absence and presence of inducer and assayed for luciferase activity (Methods). The bars on the far right correspond to a strain harboring the promoter but no TF, serving as a constitutive control. FIG. 10B shows the Pagg orthogonality test. FIG. 10C shows the Ptta orthogonality test. FIG. 10D shows the Pttg orthogonality test. FIG. 10E shows the Pgac orthogonality test. FIG. 10F shows the illustration of the 20 transcription factors used in this study. Data represent the average of n=6 biological replicates. Error bars correspond to the SEM of these measurements.

FIGS. 11A, 11B, 11C, 11D, 11E, and 11F show the transcription factor sensitivity and OD-CFU comparison. FIG. 11A shows the dose response curve for I⁺_{YQR} regulating PO1 in B. thetaiotaomicron. Cells were grown in TYG medium containing various concentrations of IPTG and assayed for luciferase activity (Methods) to assess transcription factor sensitivity. FIG. 11b shows the dose response curve for R⁺_{YQR} regulating PO1 in B. thetaiotaomicron. Cells were grown in TYG medium containing various concentrations of D-ribose and assayed for luciferase activity. FIG. 11C shows the dose response curve for I^{A}_{YQR} regulating PO1 in B. thetaiotaomicron. Cells were grown in TYG medium containing various concentrations of IPTG and assayed for luciferase activity. FIG. 11D shows the dose response curve for R^{A}_{YQR} regulating PO1 in B. thetaiotaomicron. Cells were grown in TYG medium containing various concentrations of D-ribose and assayed for luciferase activity. FIG. 11E shows the autoluminescence of wildtype Bacteroides. Each species was grown in TYG medium and assayed for luminescence (Methods). FIG. 11F shows the OD-CFU curve. An example plot of OD600 of wildtype B. thetaiotaomicron grown in TYG medium converted to colony forming units (CFU). Cultures were grown for 16 hours with samples taken at regular intervals. Samples were serially diluted and plated on BHI agar to determine CFU. For luciferase assays, data represent the average of n=6 biological replicates. Error bars correspond to the SEM of these measurements. For OD-CFU curves, data represent the average of n=3 biological replicates. Error bars correspond to the SEM of these measurements.

FIGS. 12A, 12B, 12C, and 12D show the additional single-promoter logic gates constructed in B. thetaiotaomicron. FIG. 12A shows the additional AND gates created with different promoters. Each ligand condition is compared to the ON state with the corresponding fold-change in luciferase activity shown below. Cultures were assayed as described in FIG. 2. The ADR of the transcription factors used in each circuit are shown to the right of each row. FIG. 12B shows the additional NOR gates created with different promoters. Each ligand condition is compared to the ON state with the corresponding fold-change in luciferase activity shown below. FIG. 12C shows the additional A NIMPLY B gates created with different promoters. Each ligand condition is compared to the ON state with the corresponding foldchange in luciferase activity shown below. FIG. 12D shows the additional B NIMPLY A gates created with different promoters. Each ligand condition is compared to the ON state with the corresponding fold-change in luciferase activity shown below. Data represent the average of n=6 biological replicates. Error bars correspond to the SEM of these measurements.

FIGS. 13A, 13B, and 13C show a complete set of 16 logic gates and consortium transcriptional programming charts. FIG. 12A shows the full set of 16 two-input logic gates presented for reference. Each gate has a corresponding truth table shown to the right. FIG. 13B shows the gain-of-function programming chart for co-culture. Each logic gate in FIG. 13A is listed vertically and horizontally to denote their potential use in chassis cell 1 or 2, respectively. The four input conditions (ligand combinations) are shown adjacent to the first column. A yellow box indicates that under the specified ligand condition gene expression will be activated in the corresponding chassis cell (chassis 1 is on the left and chassis 2 is on the right for each 2×4 grid). A white box indicates gene expression is off. Considering the co-culture experiment described in FIG. 6, Bo is designated as chassis cell 1 and Bt as chassis cell 2. The red rectangle thus indicates the program used in FIG. 6. FIG. 13C shows the loss-of-function programming chart for co-culture. This chart is analogous to FIG. 13B, but in the context of CRISPRi knockdown of a target gene. A blue box indicates a state of high gene expression while a white box indicates a knockdown state of gene expression. Considering the co-culture experiment described in FIG. 7, Bo is designated as chassis cell 1 and Bu as chassis cell 2. The red rectangle indicates the program used in FIG. 7.

FIGS. 14A, 14B, 14C, and 14D show the circuit compression of NIMPLY and IMPLY gates. Related to FIG. 3, degree of compression (left) is presented alongside performance of logic gates in five Bacteroides species (right). FIGS. 14A and 14B show the NIMPLY gate compression and performance in five Bacteroides. See FIG. 2 and FIGS. 15E and 15F for more details regarding gate construction. Degree of circuit compression is represented by the number of regulated promoters required to construct the logic gate. Strains harboring circuits were grown in the presence of all combinations of both inducers and assayed for luciferase activity (Methods). FIGS. 14C and 14D show the IMPLY gate compression and performance in five Bacteroides. See FIGS. 81 and 8J for details regarding gate construction. Regulated promoters used for each logic gate are shown in the bottom right corner of each left-hand box (also see FIG. 1 for more detail). Data represent the average of n=6 biological replicates. Error bars correspond to the SEM of these measurements.

FIGS. 15A, 15B, 15C, 15D, 15E, 15F, 15G, 15H, 15I, 15J, 15K, and 15L show the direct comparison of compressed circuits with Cello programming (3 pages). Wiring diagrams are presented for all logic gates constructed in this study (left) as well as the equivalent logic gates reported in Nielsen et al. 3 (right). For circuits built in this study, the promoters correspond to those shown in FIG. 1. The binary truth table for the logic gate is shown to the right. For Cello circuits, X indicates a generic TF with the subscript indicating the cognate promoter it recognizes. Only X1 and X2 are used as inducible TFs, while all other TFs are used only to repress their cognate promoters. FIG. 15A shows the BUFFER gate. FIG. 15B shows the NOT gate. FIG. 15C shows the AND gate. FIG. 15D shows the NOR gate. The reported Cello NOR gate is shown (middle) along with a theoretical NOR gate constructed without tandem promoters (right). FIG. 15E shows the A NIMPLY B gate. FIG. 15F shows the B NIMPLY A gate. FIG. 15G shows the OR gate. The reported Cello OR gate is shown (middle) along with a theoretical OR gate constructed without tandem promoters (right). h, NAND gate. FIG. 15I shows the A IMPLY B gate. FIG. 15J shows the B IMPLY A gate. FIG. 15K shows the XOR gate. An additional apparent XOR gate reported by Taketani et al. 4 is displayed at the bottom. This circuit utilizes CRISPRi and requires a constitutively expressed dCas9 gene. X1 and X2 are inducible TFs that recognize P1 and P2, respectively. The circuit relies on sgRNAs to repress synthetic promoters and utilizes two output genes to achieve apparent XOR phenotype. FIG. 15L shows the XNOR gate.

FIGS. 16A, 16B, 16C, 16D, 16E, 16F, and 16G show the demonstration of SusC knockdown in B. thetaiotaomicron. FIG. 16A shows the wiring diagram of CRISPRi circuit targeting endogenous SusC-like gene. Strains were created with X⁺_{YQR} regulating a sgRNA specific to the B. thetaiotaomicron amylopectin SusC gene. FIG. 16B shows the cartoon of PUL organization, highlighting the function of the SusC-like importer. FIG. 16C shows the growth curves of B. thetaiotaomicron harboring circuit shown in a I⁺_{YQR} as the sgRNA regulator. FIG. 16D shows the growth curves of B. thetaiotaomicron harboring circuit shown in a with R⁺_{YQR} as the sgRNA regulator. Strains harboring CRISPRi circuits were grown in the absence and presence of inducer in minimal media containing amylopectin as the only carbon source (Methods). FIG. 16E shows the wiring diagram for an AND gate controlling the sgRNA targeting B. thetaiotaomicron amylopectin SusC gene (left). Growth curves of B. thetaiotaomicron harboring this circuit when grown in amylopectin minimal media containing all combinations of both inducers (right). For OD600 growth curves, data represent the average of n=3 biological replicates. Error bars correspond to the SEM of these measurements. FIGS. 16F and 16G are related to FIG. 6. The monoculture growth curve experiments presented in FIGS. 5A and 5C were repeated using dilution plating to quantify culture density instead of OD600. Data represent the average of n=3 technical replicates. Error bars correspond to the SEM of these measurements. This provides a direct comparison to the co-culture data presented in FIG. 6.

FIG. 17 shows the maps of plasmids constructed. Plasmid names correspond to descriptions in Table 1.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more, increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred to.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “promoter,” as used herein, refers to a sequence in DNA that mediates the initiation of transcription by an RNA polymerase. Transcriptional promoters may comprise one or more of a number of different sequence elements as follows: 1) sequence elements present at the site of transcription initiation; 2) sequence elements present upstream of the transcription initiation site and; 3) sequence elements down-stream of the transcription initiation site. The individual sequence elements function as sites on the DNA, where RNA polymerases and transcription factors that facilitate positioning of RNA polymerases on the DNA bind.

A “transcription factor” refers to a sequence-specific DNA-binding protein that controls the rate of transcription of genetic information from DNA to messenger RNA, by binding to a specific DNA sequence.

As used herein, a “transcription terminator” or a “terminator” refers to a segment of a nucleic acid sequence that marks the end of gene in genomic DNA during the transcription process, or gene expression. This sequence mediates or signals the end of transcription by providing signaling nucleotides in newly synthesized RNA transcripts that trigger an RNA polymerase to release the DNA and newly synthesized RNA.

The word “vector” refers to any vehicle that carries a polynucleotide into a cell for the expression of the polynucleotide in the cell. The vector may be, for example, a plasmid, a virus, a phage particle, or a nanoparticle. A “bacterial plasmid” is a small extrachromosomal DNA molecule that can be incorporated into another cell that is physically separated from the chromosomal DNA and is easily replicated. Once transformed into a suitable host, the vector may replicate and function independently of the host genome, or may in some instances, integrate into the genome itself. In some embodiments, the vector is a DNA construct containing a DNA sequence which is operably linked to a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

An “effective amount” is an amount sufficient to affect beneficial or desired results. An effective amount can be administered in one or more administrations, applications or dosages. “Effective amount” encompasses, without limitation, an amount that can ameliorate, reverse, mitigate, prevent, or diagnose a symptom or sign of a medical condition or disorder (e.g., HIV-1 infection). Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter.

The term “microbiota” refers to the range of microorganisms that may be commensal, symbiotic, or pathogenic found in and on all multicellular organisms, including plants and animals. These include bacteria, archaea, protists, fungi, and viruses and have been found to be crucial for immunologic, hormonal, and metabolic homeostasis of the host.

As used herein, “monitoring” refers to the actions of observing and checking the progress or quality of a treatment or procedure over a period of time. Herein, “monitoring” refers to the actions of observing and checking for changes to the GI tract microbiome following administration of a cell comprising a construct to (re)program to transcriptional regulation of the microbiome.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA).

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

As used herein, “upstream” refers to the relative position of a genetic sequence, either DNA or RNA. Upstream relates to the 5′ to 3′ direction relative to the start site of transcription, wherein upstream is usually closer to the 5′ end of a genetic sequence.

As used herein, “downstream” refers to the relative position of a genetic sequence, either DNA or RNA. Downstream relates to the 5′ to 3′ direction relative the start site of transcription, wherein downstream is usually closer to the 3′ end of a genetic sequence.

“Gene” includes a nucleic acid fragment that expresses a functional molecule such as, but not limited to, a specific protein, including regulatory sequences preceding (5′ noncoding sequences) and following (3′ non-coding sequences) the coding sequence. “Native gene” refers to a gene as found in its natural endogenous location with its own regulatory sequences.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used) examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

An “enhancer” is a DNA sequence that can stimulate promoter activity and may be an innate element of the promoter or a heterologous element inserted to enhance the level or tissue-specificity of a promoter. Promoters may be derived in their entirety from a native gene or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

### Nucleic Acid Constructs and Cell Compositions

The microbial community, or microbiome, residing in the human gastrointestinal tract (GI tract) comprise a relatively undiscovered area for understanding the development of GI tract functions, and better understanding of health disorders and diseases, and development of associated treatments and therapies. Herein, it should be noted that “microbial community”, “microbiome”, and “microbiota” are used interchangeably to refer to the bacterial populations, including the bacterial organisms and the genetic material within the bacteria residing in the human GI tract. The microbiota is now recognized as a human organ comprising its own functions, including but not limited to regulating gene expression for mucosal barrier fortification, angiogenesis, and intestinal maturation. The microbiota is also involved in normal digestion and impacts the energy harvest from the diet and energy storage in the host.

The diversity of the GI tract microbiome has been revealed to be represented by over 1500 bacterial species. From birth, the normal GI tract microbiome contributes to the development of GI tract function, influences the immune system, contributes to the regulation and maintenance of the intestinal barrier, and promotes tolerance of foods. It is now recognized that a symbiotic relationship exists between human host and the microbiota that is fundamental to human health. Disruption of the stability of the GI tract microbiota is associated with, or may even contribute to the pathogenesis of diseases. Unfavorable changes to the microbiota, often caused dysregulation of microbiome gene expression, is associated with several childhood and adult diseases, including but not limited to nosocomial infections, necrotizing enterocolitis (NEC), inflammatory bowel disease (IBD), obesity, autoimmune diseases, and allergies. Because of the interactions/relationships between the human hosts and GI tract microbiota impacting human health and disease, there is a need to develop constructs, compositions, and methods for (re)programming the GI tract microbiota.

The present disclosure provides constructs and cell compositions for reprogramming a GI microbiome.

In one aspect, disclosed herein is a construct comprising a plurality of nucleic acid sequences encoding a first group of one or more regulatory core domains, a second group of one or more regulatory core domains, one or more DNA binding domains, wherein the first group of the one or more regulatory core domains and the second group of the one or more regulatory core domains are each linked to one of the DNA binding domains, and one or more DNA operator elements, wherein the one or more DNA operator elements are each specifically recognized by one of the DNA binding domains.

In some embodiments, the first group comprises 1, 2, 3, 4, 5, 6, 7, 8, 9, 10, or more regulatory core domains. In some embodiments, the second group comprises 1, 2, 3, 4, 5, 6, 7, 8, 9, 10, or more regulatory core domains. In some embodiments, construct comprises 1, 2, 3, 4, 5, 6, 7, 8, 9, 10, or more DNA binding domains.

In some embodiments, the construct further comprises SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or variants thereof.

It should be understood that that term “variant” refers to the construct having at least 50% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

Thus, in some embodiments, the construct comprises at least 50% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises at least 55% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises at least 60% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises at least 65% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises at least 70% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises at least 75% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises at least 80% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises at least 85% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises at least 90% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises at least 95% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises at least 99% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the construct comprises SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

Ins some embodiments, the plurality of nucleic acid sequences comprises any combination of SEQ ID NO: 1, SEQ ID NO: 2, SEQ ID NO: 3, SEQ ID NO: 4, SEQ ID NO: 5, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or variants thereof.

In some embodiments, the first group of one or more regulatory core domains comprises at least one repressor or at least one anti-repressor, or a combination thereof. In some embodiments, the first group of one or more regulatory core domains comprises one, two, three, four, five or more repressors or one, two, three, four, five or more anti-repressor, or a combination thereof. In some embodiments, the first group of one or more regulatory core domains comprises at least two repressor or at least two anti-repressor, or a combination thereof.

In some embodiments, the second group of one or more regulatory core domains comprise at least one repressors, at least one anti-repressors, or a combination thereof. In some embodiments, the second group of one or more regulatory core domains comprises one, two, three, four, five or more repressors or one, two, three, four, five or more anti-repressor, or a combination thereof. In some embodiments, the second group of one or more regulatory core domains comprises at least two repressor or at least two anti-repressor, or a combination thereof.

In some embodiments, the first group of one or more regulatory core domains are specifically recognized by a first agent. In some embodiments, the first agent is isopropyl-β-D-1-thiogalactopyranoside.

In some embodiments, the second group of one or more regulatory core domains are specifically recognized by a second agent. In some embodiments, the second agent is D-ribose.

In some embodiments, the first and second group of the one or more regulatory core domains are linked to a same DNA binding domain. In some embodiments, the first and second group of the one or more regulatory core domains are linked to different DNA binding domains.

In some embodiments, the construct comprises a plurality of nucleic acid sequences encoding a first group of two regulatory core domains, a second group of two regulatory core domains, three DNA binding domains, wherein the first group of the regulatory core domains and second group of the regulatory core domains are each linked to one of the three DNA binding domains, and three DNA operator elements that are each specifically recognized by one of the three DNA binding domains.

In some embodiments, the construct further comprises a nucleic acid sequence encoding a reporter including, but not limited to green fluorescent protein (GFP), yellow fluorescent protein (YFP), blue fluorescent protein (BFP), cyane fluorescent protein (CFP), monomeric red fluorescent protein (mRFP), Discosoma svriata (DsRed), mCherry, mOrange, tdTomato, mSTrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), luciferase, Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, and/or EBFP2.

In some embodiments, the construct is coupled to a nucleic acid sequence encoding components of a CRISPR gene editing system.

Clustered regularly interspaced short palindromic repeats (CRISPR) and CRISPR-associated system (CRISPR/-Cas9) is a popular tool for genome editing. However, use of CRISPR-Cas9 as a programmable genome editing tool is hindered by off-target DNA cleavage (Cong et al., 2013; Doudna, 2020; Fu et al., 2013; Jinek et al., 2013), and the underlying mechanisms by which Cas9 recognizes mismatches are poorly understood (Kim et al., 2019; Liu et al., 2020; Slaymaker and Gaudelli, 2021). Although Cas9 variants with greater discrimination against mismatches have been designed (Chen et al., 2017; Kleinstiver et al., 2016; Slaymaker et al., 2016), these suffer from significantly reduced on-target DNA cleavage rates (Kim et al., 2020; Liu et al., 2020).

In some embodiments, the construct further comprises a nucleic acid sequence encoding a dead Cas9 endonuclease (dCas9) and a single guide RNA (sgRNA).

The dCas9, also known as an endonuclease deficient Cas, is a variant form of the parent Cas9, whose endonuclease activity is removed by mutating the endonuclease domains. It should be understood however that dCas9 may still possess binding activity to guide RNA and targeted DNA strands.

Disclosed herein is an isolated Cas9 variant or a fragment. By “variant” or “fragment” is meant a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, or at least 99% sequence identity to a parent Cas9 polypeptide. It is noted that “parent” and “native” are referred to alternatively herein, and have the same meaning, which is the naturally occurring Cas9 on which the variant or fragment thereof is based.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the CRISPR/Cas system that can form a complex with a Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

In some embodiments, the nucleic acid sequence encodes any combination of SEQ ID NO: 6, SEQ ID NO: 16, SEQ ID NO: 17, SEQ ID NO: 18, SEQ ID NO: 19, or variants thereof.

In some embodiments, the construct further comprises at least 50% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises at least 55% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises at least 60% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises at least 65% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises at least 70% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises at least 75% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises at least 80% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises at least 85% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises at least 90% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises at least 95% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises at least 99% sequence identity to SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 81.

In some embodiments, the construct further comprises SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, or variants thereof.

In one aspect, disclosed herein is a cell comprising a construct comprising a plurality of nucleic acid sequences encoding a first group of one or more regulatory core domains, a second group of one or more regulatory core domains, one or more DNA binding domains, wherein the first group of the one or more regulatory core domains and the second group of the one or more regulatory core domains are each linked to one of the DNA binding domains, and one or more DNA operator elements, wherein the one or more DNA operator elements are each specifically recognized by one of the DNA binding domains.

In some embodiments, the cell comprises the construct of any preceding aspect.

It should be understood that the construct can be introduced and/or integrated into the cell by techniques commonly known in the art including, but not limited to the method of transformation. “Transformation” of a cellular organism with DNA means introducing DNA into an organism so that at least a portion of the DNA is replicable, either as an extrachromosomal element or by chromosomal integration. The term “transformed” refers to a cell in which DNA was introduced. The cell is termed “host cell” and it may be either prokaryotic or eukaryotic. Typical prokaryotic host cells include various strains of E. coli. Typical eukaryotic host cells are mammalian, such as gastrointestinal cells of human origin. The introduced DNA sequence may be from the same species as the host cell or a different species from the host cell, or it may be a hybrid DNA sequence, containing some foreign and some homologous DNA.

In some embodiments, the cell is a bacterial cell. In some embodiments, the bacterial cell is a bacterium of gastrointestinal tract microbiota. In some embodiments, the bacterial cell is a species of Bacteroides genus selected from B. thetaiotaomicron (Bt), B. fragilis (Bf), B. vulgatus (Bv), B. ovatus (Bo), or B. uniformis (Bu).

### Methods

The present disclosure also provides methods of using nucleic acid constructs and/or cell compositions to modify and/or monitor a GI microbiome.

In one aspect, disclosed herein is a method of modifying a gastrointestinal tract microbiome in a subject, comprising administering to the subject an effective amount of a cell comprising a construct comprising a plurality of nucleic acid sequences encoding a first group of one or more regulatory core domains, a second group of one or more regulatory core domains, one or more DNA binding domains, wherein the first group of the one or more regulatory core domains and the second group of the one or more regulatory core domains are each linked to one of the DNA binding domains, and one or more DNA operator elements, wherein the one or more DNA operator elements are each specifically recognized by one of the DNA binding domains.

In some embodiments, the method of modifying a gastrointestinal tract microbiome in a subject, comprising administering to the subject an effective amount of the cell of any preceding aspect, wherein the cell comprises the construct of any preceding aspect.

As used herein, “modifying a gastrointestinal tract microbiome” refers to transcriptionally increasing or decreasing functions, cell numbers, or combinations thereof in a host organism, such as humans, to promote or revert the host GI tract to a normal functioning state. The method of modifying a GI tract microbiome” also refers to transcriptionally increasing or decreasing functions, cell numbers, gene expression, or combinations thereof in a host organism to facilitate the understanding of disease pathogeneses associated with the GI tract and further understanding bacterial populations within the GI tract microbiome.

In one aspect, disclosed herein is a method of monitoring a gastrointestinal tract microbiome in a subject, comprising administering to the subject an effective amount of a cell comprising a construct comprising a plurality of nucleic acid sequences encoding a first group of one or more regulatory core domains, a second group of one or more regulatory core domains, one or more DNA binding domains, wherein the first group of the one or more regulatory core domains and the second group of the one or more regulatory core domains are each linked to one of the DNA binding domains, and one or more DNA operator elements, wherein the one or more DNA operator elements are each specifically recognized by one of the DNA binding domains.

In some embodiments, the method of monitoring a gastrointestinal tract microbiome in a subject, comprising administering to the subject an effective amount of the cell of any preceding aspect, wherein the cell comprises the construct of any preceding aspect.

As used herein, “monitoring a gastrointestinal tract microbiome” refers to the processes of observing and/or routinely checking the increases or decreases in functions, cell numbers, or combinations thereof caused by transcriptionally (re)programming a host microbiome. It should be understood that the process of monitoring can be performed as often or as sparingly necessary to observe a desired effect. In some embodiments, the host can be monitored every day, every 2 days, every 3 days, every 4 days, every 5 days, every 6 days, every 7 days, or more. In some embodiments, the host can be monitored every week, every 2 weeks, every 3 weeks, every 4 weeks, or more. In some embodiments, the host can be monitored every month, every 2 months, every 3 months, every 4 months, every 5 months, every 6 months, every 7 months, every 8 months, every 9 months, every 10 months, every 11 months, every 12 months, or more. In some embodiments, the host can be monitored every year, every 2 years, every 3 years, every 4 years, every 5 years, or more.

In some embodiments, the host can be monitored 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times.

In one aspect, disclosed herein is a method of treating or preventing a disease or disorder in a subject in need thereof, the method comprising administering to the subject an effective amount of a cell comprising a construct comprising a plurality of nucleic acid sequences encoding a first group of one or more regulatory core domains, a second group of one or more regulatory core domains, one or more DNA binding domains, wherein the first group of the one or more regulatory core domains and the second group of the one or more regulatory core domains are each linked to one of the DNA binding domains, and one or more DNA operator elements, wherein the one or more DNA operator elements are each specifically recognized by one of the DNA binding domains, and wherein the construct transcriptionally (re)programs a bacterial population within the subject's GI tract to improve the host's health.

In one aspect, disclosed herein is a method of treating or preventing a disease or disorder in a subject in need thereof, the method comprising administering to the subject an effective amount of the cell comprising the construct of any preceding aspect, wherein the construct transcriptionally (re)programs a bacterial population within the subject's GI tract to improve the host's health.

In some embodiments, the method (re)programs the bacterial population into a therapeutic bacteria. In some embodiments, the bacterial population comprises a Bacteroides species including, but not limited to B. thetaiotaomicron (Bt), B. fragilis (Bf), B. vulgatus (Bv), B. ovatus (Bo), or B. uniformis (Bu).

In some embodiments, the disease or disorder includes, but are not limited to a cancer, a gastrointestinal disease, a congenital disease or disorder, an infectious disease, or combinations thereof.

In some embodiments, the cancer includes, but is not limited to acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, familiar hypereosinophilia, gall bladder cancer, gastric cancer (e.g., stomach adenocarcinoma), gastrointestinal stromal tumor (GIST), oral cancer (e.g., oral squamous cell carcinoma (OSCC), throat cancer (e.g., laryngeal cancer, pharyngeal cancer, nasopharyngeal cancer, oropharyngeal cancer)), a one or more leukemias and/or lymphomas known in the art, multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, the gastrointestinal disease includes, but is not limited to heartburn, irritable bowel syndrome, lactose intolerance, gallstones, cholecystitis, cholangitis, anal fissure, hemorrhoids, proctitis, colon polyps, infective colitis, ulcerative colitis, ischemic colitis, Crohn's disease, radiation colitis, celiac disease, diarrhea (chronic or acute), constipation (chronic or acute), diverticulosis, diverticulitis, acid reflux (gastroesophageal reflux (GER) or gastroesophageal reflux disease (GERD)), Hirschsprung disease, abdominal adhesions, achalasia, acute hepatic porphyria (AHP), anal fistulas, bowel incontinence, centrally mediated abdominal pain syndrome (CAPS), clostridioides difficile infection, cyclic vomiting syndrome (CVS), dyspepsia, eosinophilic gastroenteritis, globus, inflammatory bowel disease, malabsorption, scleroderma, volvulus, and other gastrointestinal diseases.

In some embodiments, the congenital disease or disorder includes, but is not limited to amniotic band syndrome, Angelman syndrome, Barth syndrome, chromosomal abnormalities (including, but not limited to abnormalities to chromosome 9, 10, 16, 18, 20, 21, 22, X chromosome, and Y chromosome), congenital adrenal hyperplasia, congenital hyperinsulinism, congenital sucrase-isomaltase deficiency (CSID), cystic fibrosis, De Lange syndrome, fetal alcohol syndrome, first arch syndrome, gestational diabetes, Haemophilia, heterochromia, Jacobsen syndrome, Katz syndrome, Klinefelter syndrome, Kabuki syndrome, Kyphosis, Larsen syndrome, Laurence-Moon syndrome, macrocephaly, Marfan syndrome, microcephaly, Nager's syndrome, neonatal jaundice, neurofibromatosis, Noonan syndrome, Pallister-Killian syndrome, Pierre Robin syndrome, Poland syndrome, Prader-Willi syndrome, Rett syndrome, sickle cell disease, Smith-Lemli-Optiz syndrome, spina bifida, congenital syphilis, teratoma, Treacher Collins syndrome, Turner syndrome, Umbilical hernia, Usher syndrome, Waardenburg syndrome, Werner syndrome, Wolf-Hirschhorn syndrome, Wolff-Parkinson-White syndrome, and other congenital diseases or disorders.

In some embodiments, the infectious disease includes, but is not limited to common cold, influenza (including, but not limited to human, bovine, avian, porcine, and simian strains of influenza), measles, acquired immune deficiency syndrome/human immunodeficiency virus (AIDS/HIV), anthrax, botulism, cholera, campylobacter infections, chickenpox, chlamydia infections, cryptosporidosis, dengue fever, diphtheria, hemorrhagic fevers, Escherichia coli (E. coli) infections, ehrlichiosis, gonorrhea, hand-foot-mouth disease, hepatitis A, hepatitis B, hepatitis C, legionellosis, leprosy, leptospirosis, listeriosis, malaria, meningitis, meningococcal disease, mumps, pertussis, polio, pneumococcal disease, paralytic shellfish poisoning, rabies, rocky mountain spotted fever, rubella, salmonella, shigellosis, small pox, syphilis, tetanus, trichinosis (trichinellosis), tuberculosis (TB), typhoid fever, typhus, west nile virus, yellow fever, yersiniosis, and zika.

In some embodiments, the cell of any preceding aspect or the construct of any preceding aspect is administered in combination with a therapeutic agent. In some embodiments, the therapeutic agent includes, but is not limited to an antibiotic, a probiotic, an anti-inflammatory compound, a vitamin, a mineral, or combinations thereof.

In some embodiments, the antibiotic includes, but is not limited to penicillins (including, but not limited to amoxicillin, clavulanate and amoxicillin, ampicillin, dicloxacillin, oxacillin, and penicillin V potassium), tetracyclines (including, but not limited to demeclocycline, doxycycline, eravacycline, minocycline, omadacycline, sarecycline, and tetracycline), cephalosporins (cefaclor, cefadroxil, cefdinir, cephalexin, cefprozil, cefepime, cefiderocol, cefotaxime, cefotetan, ceftaroline, cefazidme, ceftriaxone, and cefuroxime), quinolones (also referred to as fluoroquinolones include, but are not limited to ciprofloxacin, delafloxacin, levofloxacin, moxifloxacin, and gemifloxacin), lincomycins (including clindamycin and lincomycin), macrolides (including, but not limited to azithromycin, clarithromycin, erythromycin, and fidaxomicin (ketolide)), sulfonamides (including sulfamethoxazole and trimethoprim, and sulfasalazine), glycopeptides (including, but not limited to dalbavancin, oritavancin, telavancin, and vancomycin), aminoglycosides (including, but not limited to gentamicin, tobramycin, and amikacin), carbapenems (including, but not limited to imipenem and cilastatin, meropenem, and ertapenem), and topical antibiotics (including, but not limited to neomycin, bacitracin, polymyxin B, and praxomine) used alone or in combination.

In some embodiments, the probiotic comprises a food or supplement comprising a beneficial bacterial species including, but not limited to Bifidobacteria animalis, Bifidobacteria breve, Bifidobacteria bifidum, Bifidobacteria lactis, Bifidobacteria longum, Lactobcillus acidophilus, Lactobacillus reuteri, Lacticaseibacillus rhamnosus, Lacticaseibacillus casei, Lactiplantibacillus plantarum, Ligilactobacillus salivarius, Limosilactobacillus fermentum, Lactobacillus paracasei, Lactobacillus gasseri, Lactobacillus acidophilus, Saccharomyces boulardii, Limosilactobacillus reuteri, Bacillus coagulans, or Streptococcus thermophilus alone or in combination.

In some embodiments, the anti-inflammatory compound includes, but is not limited to a non-steroidal anti-inflammatory compound including, but is not limited to aspirin, ibuprofen, ketoprofen, naproxen, steroids, glucocorticoids (including, but not limited to betamethasone, budesonide, dexamethasone, hydrocortisone, hydrocortisone acetate, methylprednisolone, prednisolone, prednisone, and triamcinolone), methotrexate, sulfasalazine, lefunomide, anti-Tumor Necrosis Factor (TNF) medications, cyclophosphamide, and mycophenolate used alone or in combination.

In some embodiments, the vitamin or mineral includes, but are not limited to vitamin D, magnesium, vitamin K, vitamin A, riboflavin, vitamin B12, thiamine, zinc, vitamin B6, biotin, vitamin C, folic acid, vitamin B3, calcium, iron, or derivatives thereof, given alone or in combination.

In some embodiments, the cell of any preceding aspect or the construct of any preceding aspect is administered in combination with a life-style change including, but not limited to dietary changes, exercise, physical therapy, or combinations thereof.

In one aspect, disclosed herein is a nucleic acid construct or cell of any preceding aspect and a pharmaceutically acceptable carrier selected from an excipient, a diluent, a salt, a buffer, a stabilizer, a lipid, an emulsion, and a nanoparticle. One or more active agents (e.g. the nucleic acid construct) can be administered in the “native” form, if desired in the form of salts, esters, amides, prodrugs, a derivative that is pharmacologically suitable, or within a transformed cell. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

The cell comprising the construct or the native construct may be administered in such amounts, time, and route deemed necessary in order to achieve the desired result. The exact amount of the cell comprising the construct or the native construct will vary from subject to subject, depending on the species, age, and general condition of the subject, the severity of the disease or disorder, the particular composition, its mode of administration, its mode of activity, and the like. The cell comprising the construct or the native construct is preferably formulated in dosage unit form for ease of administration and uniformity of dosage. It will be understood, however, that the total daily usage of the cell comprising the construct or the native construct will be decided by the attending physician within the scope of sound medical judgment. The specific therapeutically effective dose level for any particular subject will depend upon a variety of factors including the disease or disorder being treated and the severity of the disease or disorder; the activity of the cell comprising the construct or the native construct employed; the specific cell comprising the construct or the native construct employed; the age, body weight, general health, sex and diet of the patient; the time of administration, route of administration, and rate of excretion of the specific cell comprising the construct or the native construct employed; the duration of the treatment; drugs used in combination or coincidental with the specific cell comprising the construct or the native construct employed; and like factors well known in the medical arts.

The cell comprising the construct or the native construct may be administered by any route deemed appropriate to achieved the desired effect. In some embodiments, the cell comprising the construct or the native construct is administered via a variety of routes, including oral, intravenous, intramuscular, intra-arterial, intramedullary, intrathecal, subcutaneous, intraventricular, transdermal, intradermal, rectal, intravaginal, intraperitoneal, mucosal, nasal, buccal, enteral, sublingual; by intratracheal instillation, or bronchial instillation. In general, the most appropriate route of administration will depend upon a variety of factors including the nature of the cell comprising the construct or the native construct (e.g., its stability in the environment of the gastrointestinal tract), the condition of the subject (e.g., whether the subject is able to tolerate the chosen route of administration), etc.

The exact amount of the cell comprising the construct or the native construct required to achieve a therapeutically or prophylactically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Transcriptional Programming in a Bacteroides consortium

Bacteroides species are prominent members of the human gut microbiota. The prevalence and stability of Bacteroides in humans make them ideal candidates to engineer as programmable living therapeutics. Herein, a biotic decision-making technology is reported in a community of Bacteroides (consortium transcriptional programming) with genetic circuit compression. Circuit compression requires systematic pairing of engineered transcription factors with cognate regulatable promoters. In turn, the compression workflow is demonstrated by designing, building, and testing all fundamental two-input logic gates dependent on the inputs isopropyl-β-D-1-thiogalactopyranoside and D-ribose. Complete sets of logical operations were deployed in five human donor Bacteroides, with which sequential gain-of-function control is demonstrated in co-culture. Finally, transcriptional programs are coupled with CRISPR interference to achieve loss-of-function regulation of endogenous genes—demonstrating complex control over community composition in co-culture. This work provides a powerful toolkit to program gene expression in Bacteroides for the development of bespoke therapeutic bacteria.

### Introduction

The human gastrointestinal (GI) tract harbors a microbial ecosystem of enormous complexity that contributes significantly to the health of the host. Evidence continues to emerge connecting the GI microbiota with health and disease states not only in the immediate vicinity of the GI tract, but systemically as well. Many studies involving the GI microbiota leverage metagenomic data to investigate how its highly variable composition across age and demographics can be connected to health conditions. In contrast, several studies have investigated the impact of individual species on the microbiota through functional genomics and targeted manipulation of GI communities. The vast majority of microbes inhabiting the GI tract are obligate anaerobes that are not readily amenable to genetic manipulation. This poses a challenge to synthetic biologists who seek to reprogram these microbes to perform useful functions beyond native capabilities. Bacteroides spp. have emerged as promising chassis cells for genetic engineering as a result of knowledge gained over several decades of studies. Their long-term stability in the human colon make Bacteroides attractive candidates for engineering as therapeutic bacteria to modulate their host's immune system by executing bespoke genetic programs, in addition to facilitating the programmed delivery of therapeutic payloads. While living therapeutics have been developed using bacteria such as Escherichia coli (E. coli) Nissle 1917 and Lactococcus lactis, these strains are typically cleared from the host within days to weeks, limiting their long-term utility. Accordingly, there is an impetus to develop a universal programming structure in Bacteroides for use as complex diagnostic tools, living-therapeutics, or for the study of these important contributors to the human microbiota, as Bacteroides can function for months to years in situ. Recent efforts have focused on developing genetic regulatory tools specifically for Bacteroides thetaiotaomicron (B. thetaiotaomicron), as parts developed in E. coli tend to be incompatible with the transcription-translation machinery of Bacteroides. With the intent of engineering select Bacteroides as putative chassis cells for further development and study, a small number of inducible promoters regulated by transcription factors have been reported, as well as promoters regulated by dCas9-sgRNA repression. Notably, Cello genetic circuit design software was recently implemented in B. thetaiotaomicron, demonstrating that higher-order transcriptional logic could be achieved in this chassis cell.

It was recently reported that the partial development of an application-agnostic decision-making technology (transcriptional programming) deployed in E. coli that leverages systems of engineered transcription factors and accompanying non-natural regulated promoters (See, Rondon et al. Transcriptional programming using engineered systems of transcription factors and genetic architectures. Nat Commun 10, 4784 (2019) and Groseclose et al. Engineered systems of inducible anti-repressors for the next generation of biological programming. Nat Commun 11, 4440 (2020)). Herein, it is reported that the transference of transcriptional programming and the development of all 16 fundamental logical operations in B. thetaiotaomicron in addition to four additional Bacteroides species (B. fragilis, B. ovatus, B. uniformis, and B. vulgatus) forms a programmable Bacteroides consortium. By combining networks of BUFFER and NOT gates in the form of single transcription factors, all 16 two-input logic gates were systematically constructed regulating a luciferase output—representing a gain-of-function programming structure. Compared to state-of-the-art genetic circuits with similar control features, the logic gates reported here are notably compressed in terms of regulated promoters and genetic parts required to build them—while possessing high performance in terms of dynamic range. In addition, the transcriptional programming system was coupled with CRISPR interference (CRISPRi) to extend control to both heterologous and endogenous genes—i.e., as a programmable loss-of-function (knockdown) technology. Moreover, said transcriptional programming technologies were deployed in co-culture to form concurrent, asymmetric, and sequential decision-making within consortia of chassis cells. First, the utility of a set of non-congruent transcriptional programs paired with CRISPRi in a simple consortium demonstrated the ability to regulate the asymmetric fitness of individual species in co-culture. In turn, sequential asymmetric programming to confer gain-of-function in a separate consortium was achieved. The consortium-based transcriptional programming framework presented here serves as a foundation for next-generation living therapeutics, and provides a powerful technology to advance the general study of the Bacteroides genus.

### Results

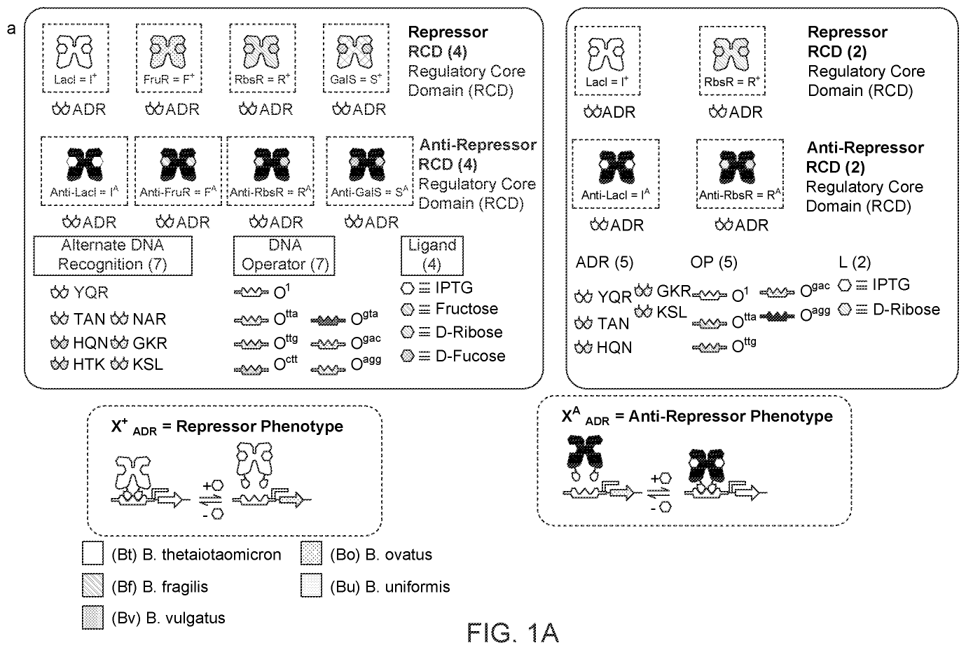

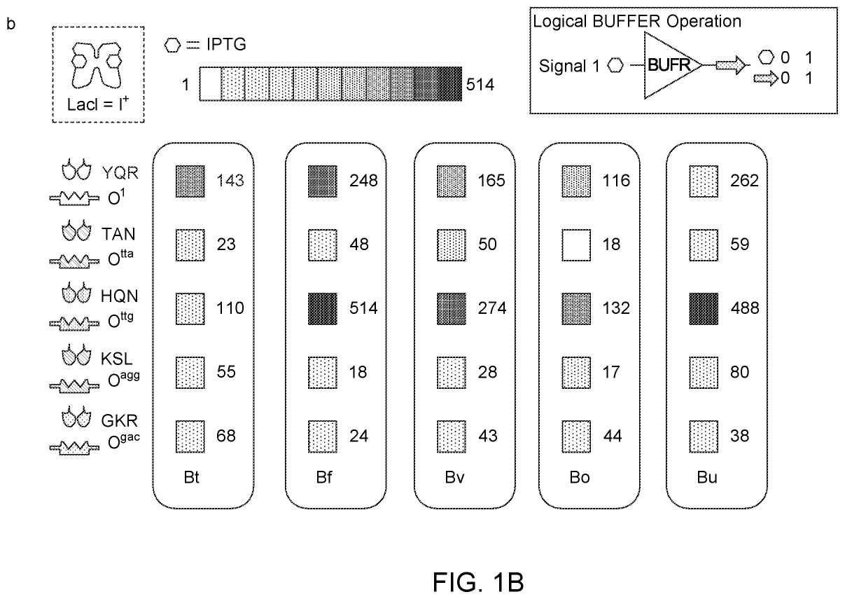

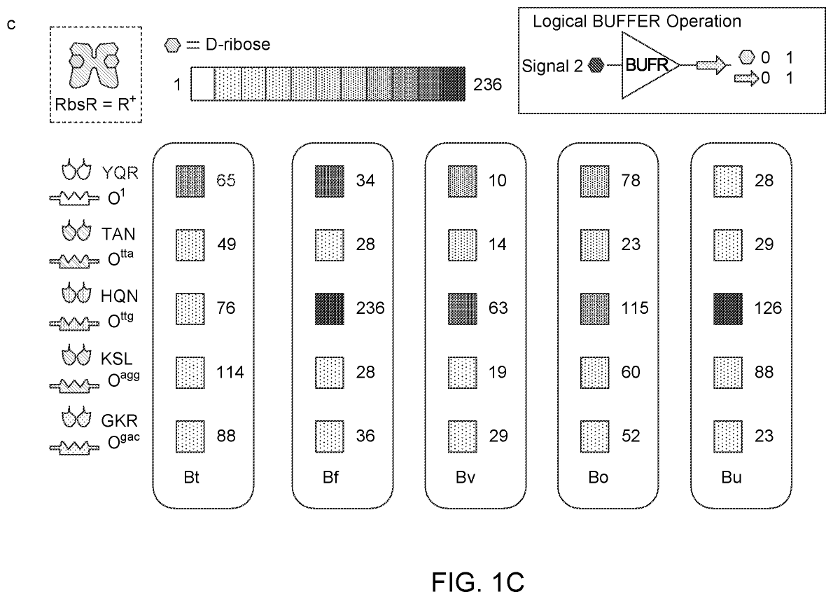

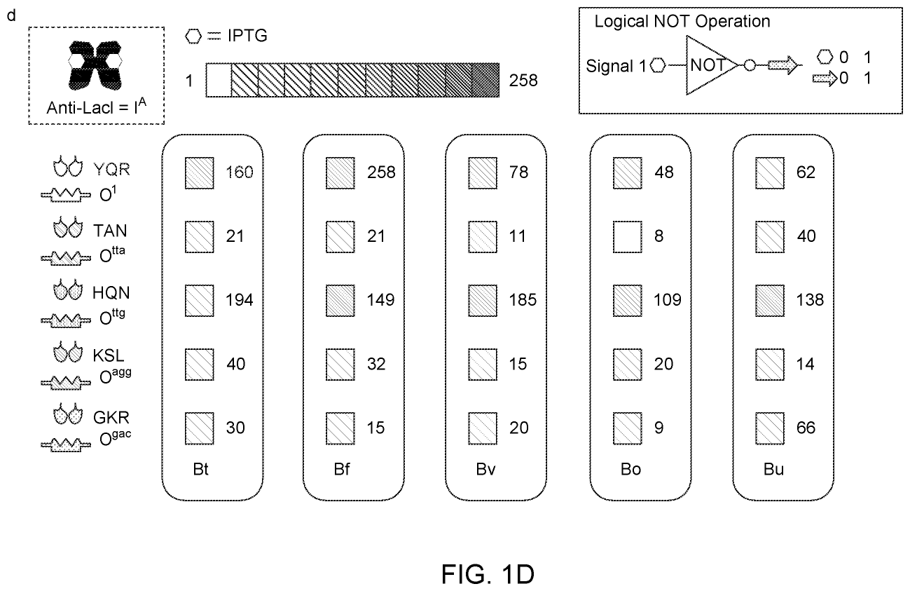

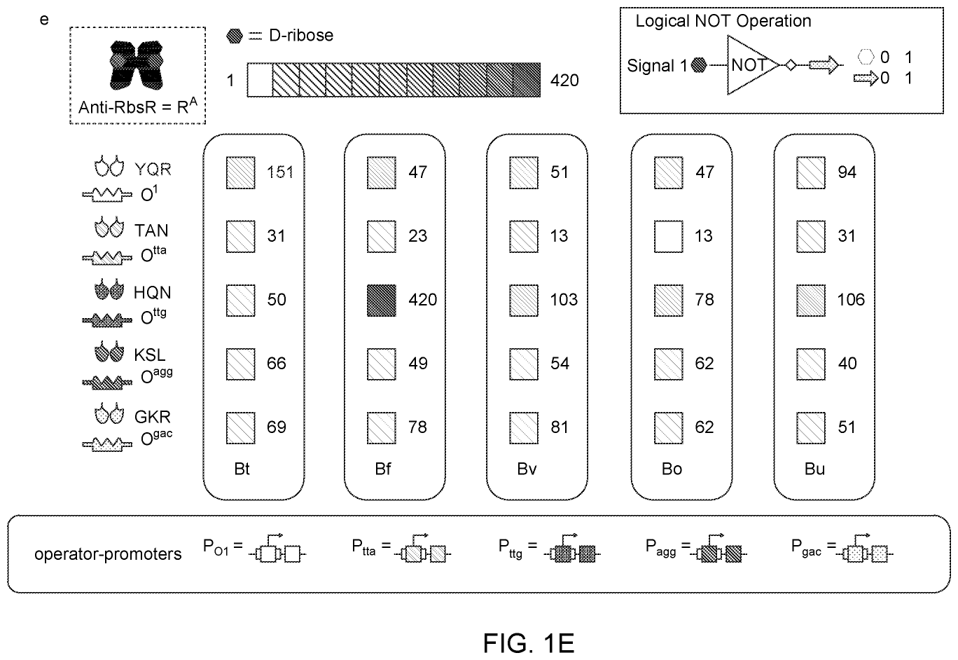

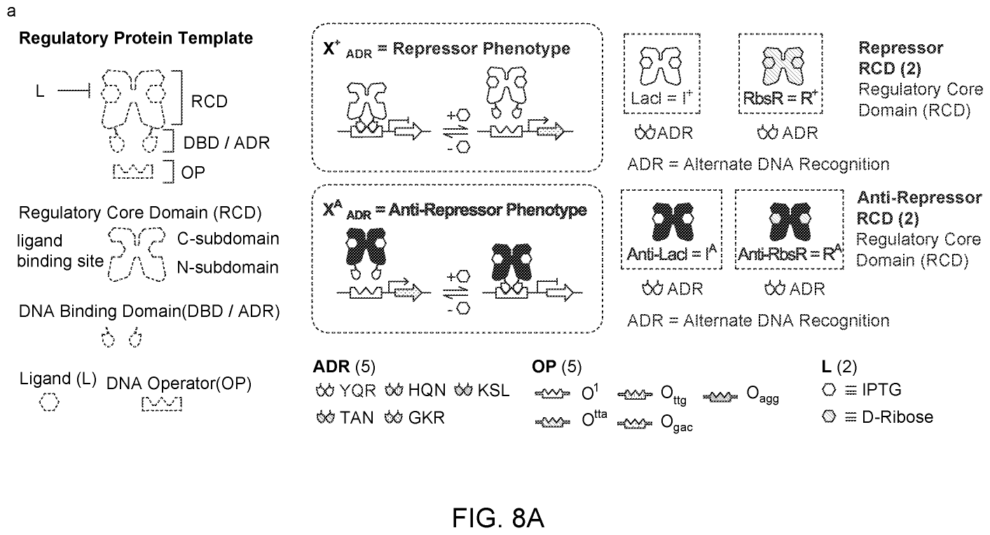

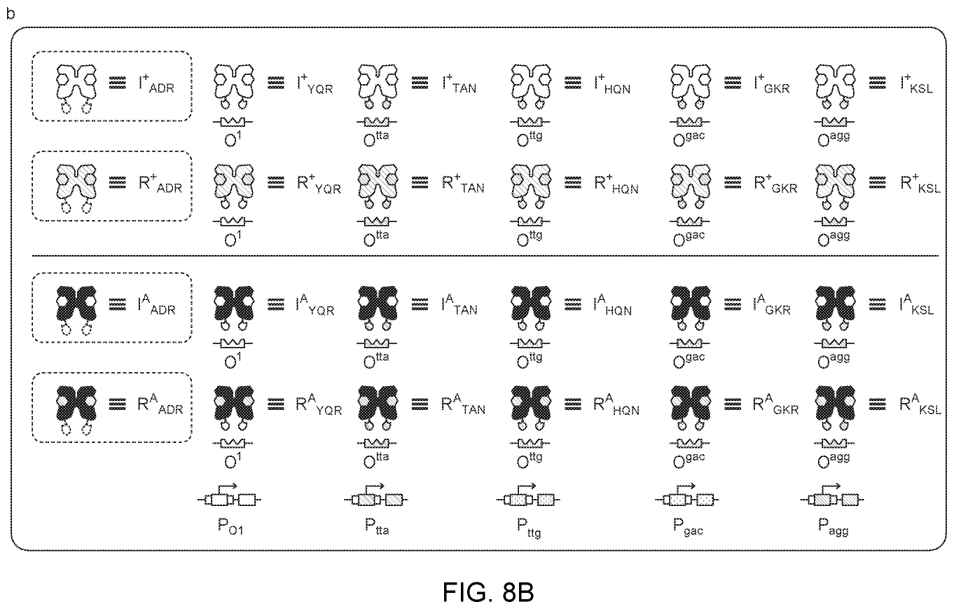

Conferring repression and complementary anti-repression in B. thetaiotaomicron using engineered transcription factors. In previous studies, four sets of signal-distinct repressors and complementary anti-repressors were used—based on the LacI/GalR topology—that could be directed to seven independent promoters in E. coli. (see FIG. 1A). This collection of engineered transcription factors (TFs) resulted in 56 single-input logical operations constructed via the systematic pairing of a transcription factor and cognate DNA operator-promoter element—i.e., 28 BUFFER gates and 28 NOT gates. Each repressor and cognate operator-promoter can be regarded as a BUFFER logical operation, whereas each anti-repressor and corresponding genetic element can be regarded as a NOT logical operation. Previous studies demonstrated that the LacI transcription factor (i.e., structural and mechanistic design template) was functional in B. thetaiotaomicron. Accordingly, many (if not all) of the 56 logical operations developed and tested in E. coli were posited to be functional in the B. thetaiotaomicron chassis cell. The goal herein was to identify at least two sets of repressors and complementary anti-repressors to facilitate the full development of transcriptional programming (i.e., via the demonstration of the systematic design, build, and test of all 16 fundamental two-input logical operations) in the B. thetaiotaomicron chassis cell. Given that two sets of engineered transcription factors—i.e., LacI (I⁺_{ADR})+anti-LacI (I^{A}_{ADR}), and RbsR (R⁺_{ADR})+anti-RbsR (R^{A}_{ADR})—resulted in significant performance in E. coli the search was focused on the best performing subset of logical operations, see FIG. 8 and FIG. 1A for a full description of nomenclature and description of the complete set of transcription factors. In brief, 5 out of the 7 alternate DNA binding recognition (ADR) functions (i.e., ADR=YQR, TAN, HQN, GKR or KSL) for said transcription factors and cognate operator-promoters resulted in greater than 20-fold dynamic range in E. coli—i.e., resulting in a putative set of 20 non-synonymous BUFFER gates, and a set of 20 non-synonymous NOT gates targeted for development in the B. thetaiotaomicron chassis cell.

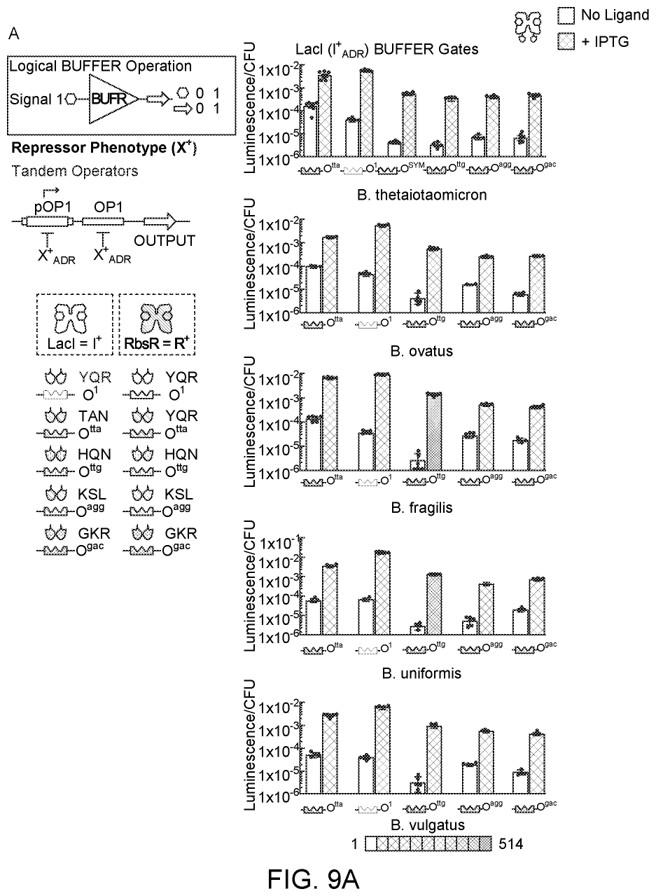

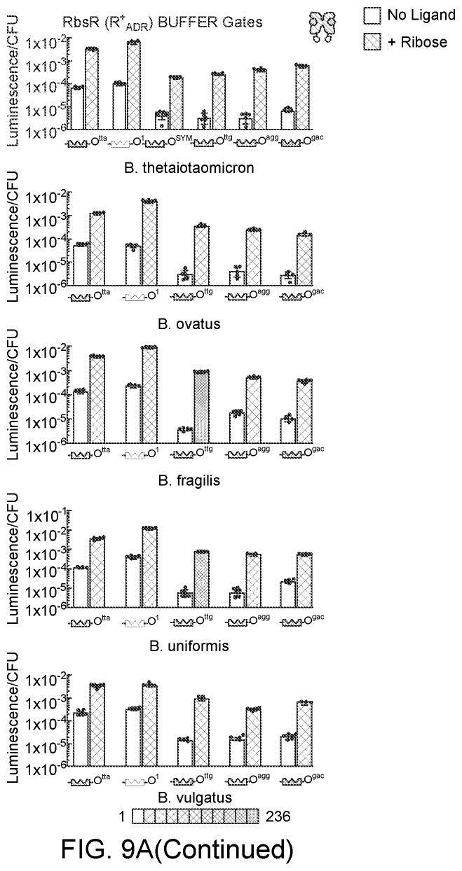

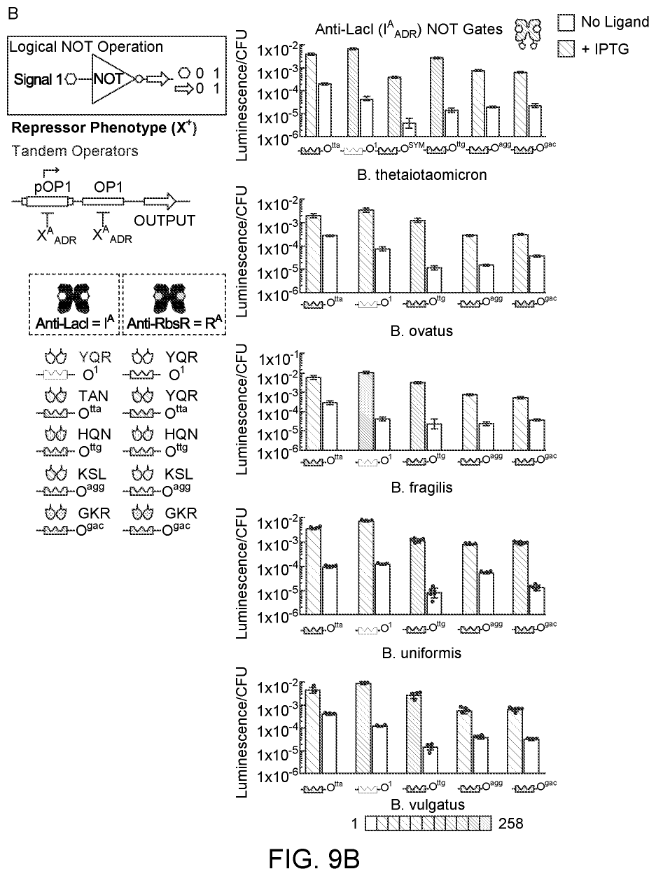

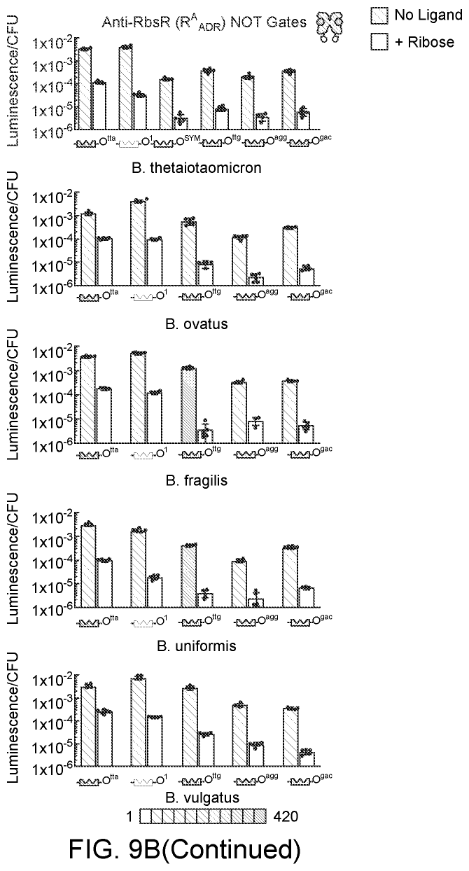

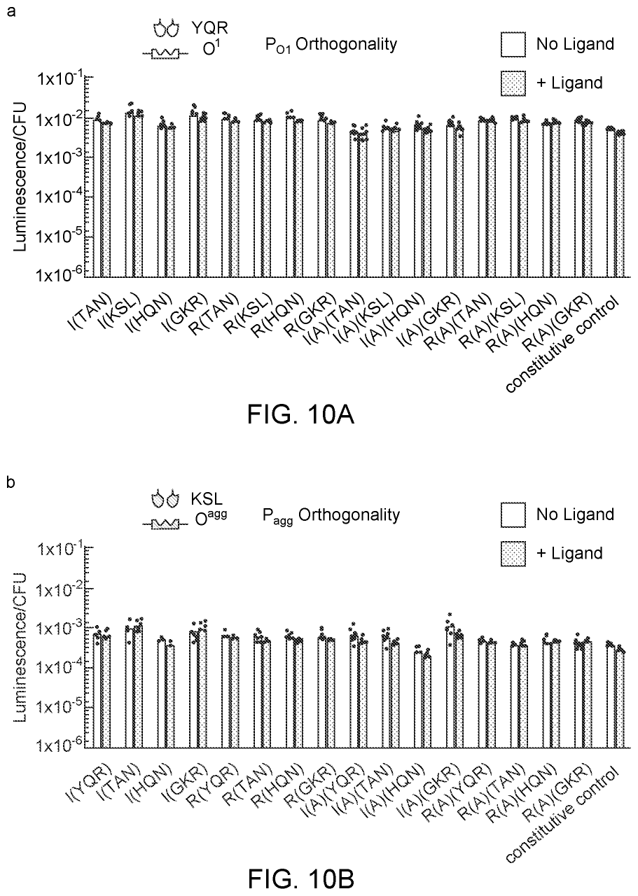

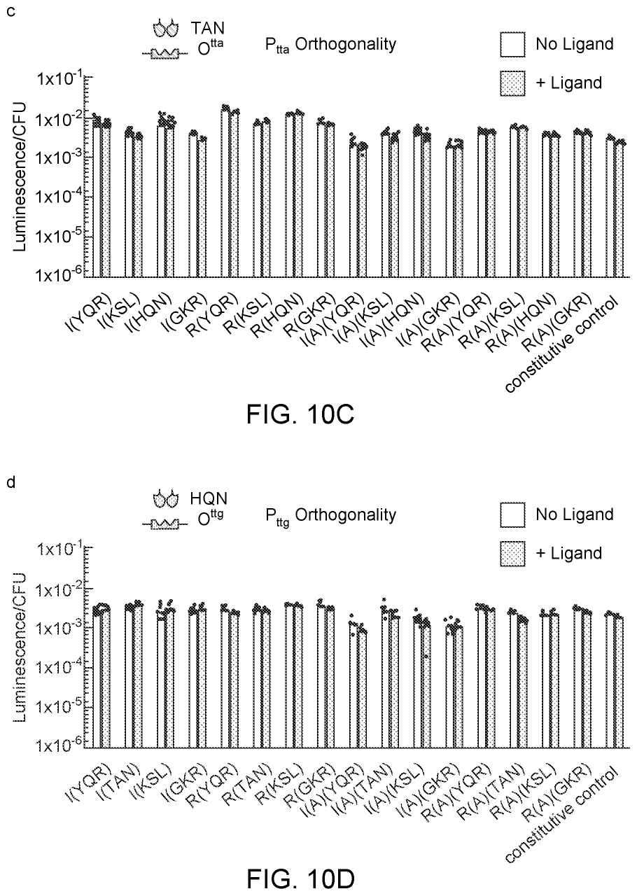

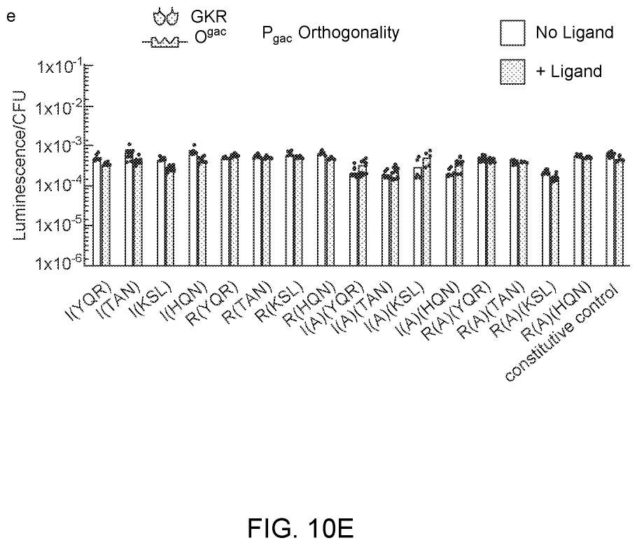

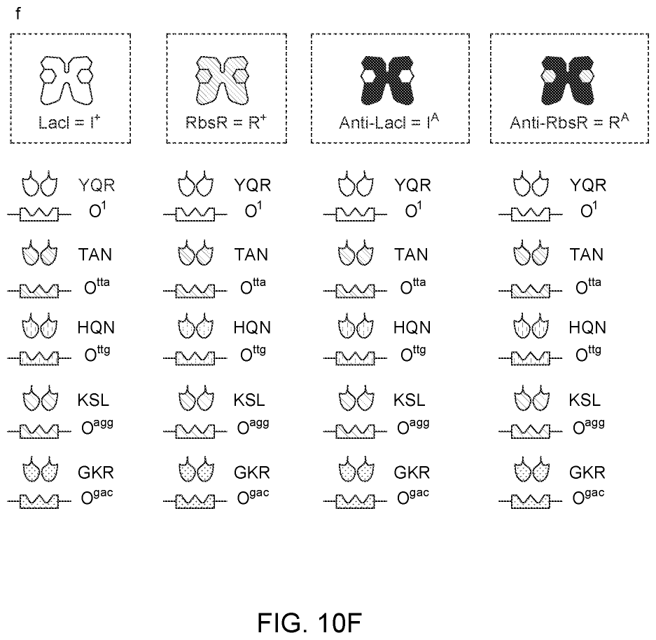

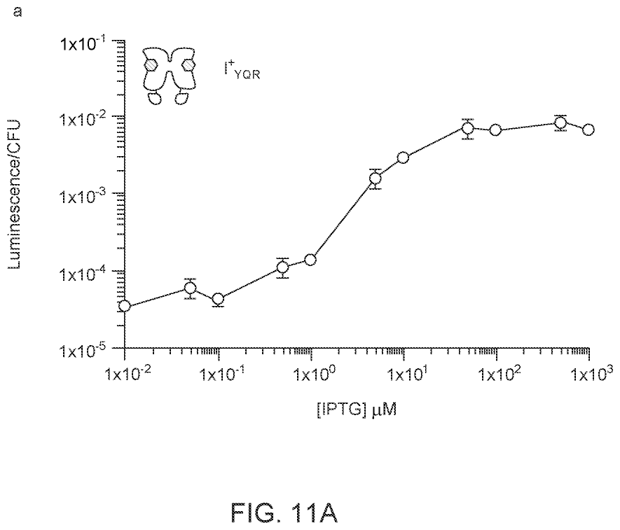

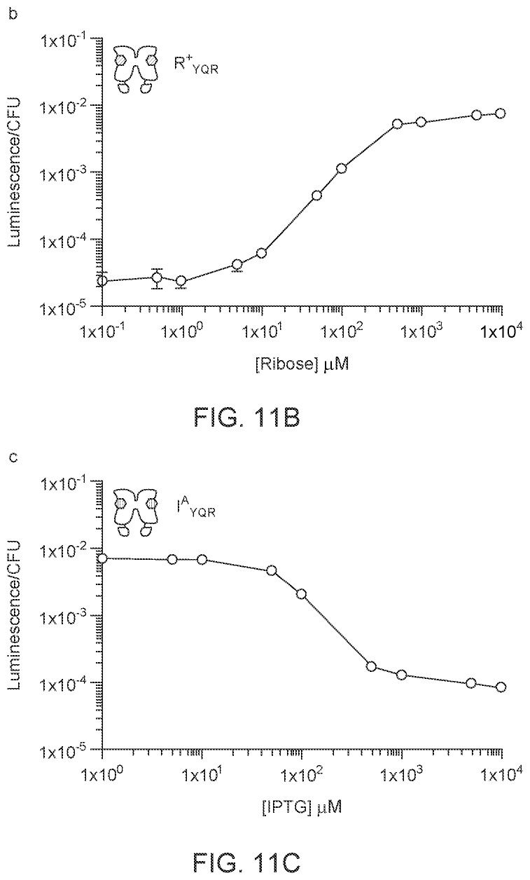

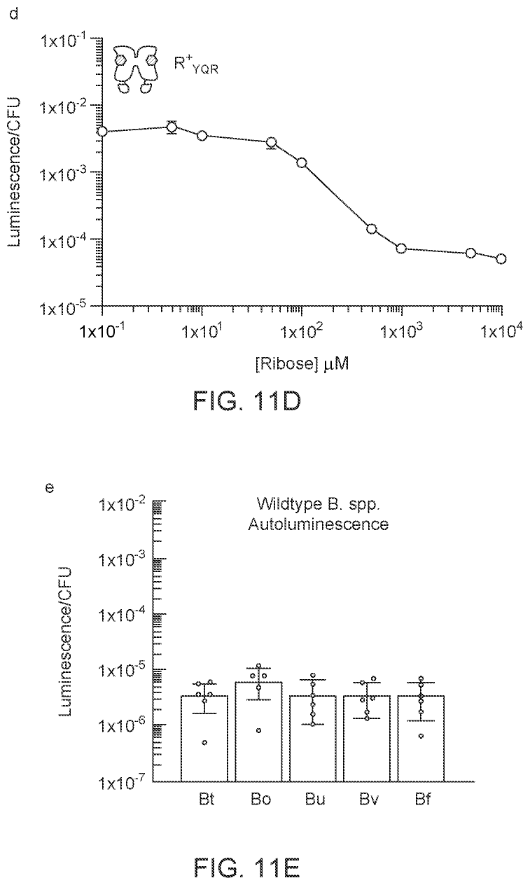

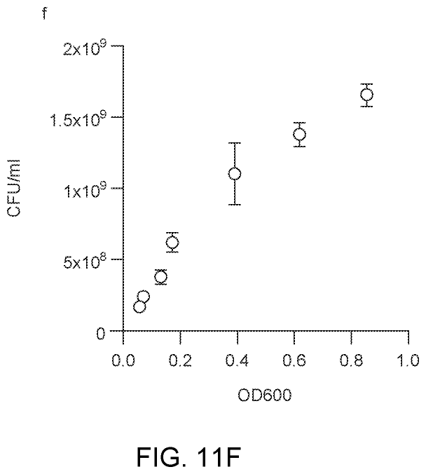

Initially, each operation was designed, built, and tested as a standard single-operator promoter system in B. thetaiotaomicron. In addition, given that common reporters like green fluorescent protein are not amenable to maturation in anaerobic environments used to culture B. thetaiotaomicron, NanoLuc luciferase was used as the regulated gene output interface. Most (>80%) of the transcription factors displayed inadequate fold-changes as single-operator promoter systems, regardless of the placement of the operator—i.e., whether at the core or proximal position alone (see Example 2). Because each DNA operator-promoter was restricted to a single (genome integrated) copy, it was contemplated that the apparent affinity for protein-DNA interaction was affected. In turn, an in-tandem operator-promoter was leveraged in which two DNA operators were used, one intercalated between the −33 and −7 hexamer and the other proximal to the transcription start site (TSS) (FIG. 1 inset, FIG. 9, and Example 2). Using this alternate architecture to direct the cognate transcription factors resulted in the identification of two sets of complementary logical operations with satisfactory performance metrics (i.e., dynamic range greater than 20). Briefly, in the B. thetaiotaomicron chassis cell 5 BUFFER gates were identified that responsive to isopropyl-β-D-1-thiogalactopyranoside (IPTG) (FIG. 1B), 5 BUFFER gates responsive to D-ribose (FIG. 1C), 5 NOT gates responsive to IPTG (FIG. 1D), and 5 NOT gates responsive to D-ribose (FIG. 1E). Notably, each set of transcription factors for a given logical operation could be independently directed to five separate cognate operator-promoters—i.e., P_{O1}, Pₜₜₐ, P_{ttg}, P_{agg}, or P_{gac}—without cross interaction (see FIG. 1 and FIG. 10). In addition, the dose response of I⁺_{YQR}, R⁺_{YQR}, I^{A}_{YQR}, and R^{A}_{YQR} was tested to verify the ligand concentrations that correlated to the ON-states and OFF-states for repressors and anti-repressors, respectively (FIG. 11). Achieving this milestone facilitated the systematic transition from single-input logical operations to two-input (one layer) logical operations.

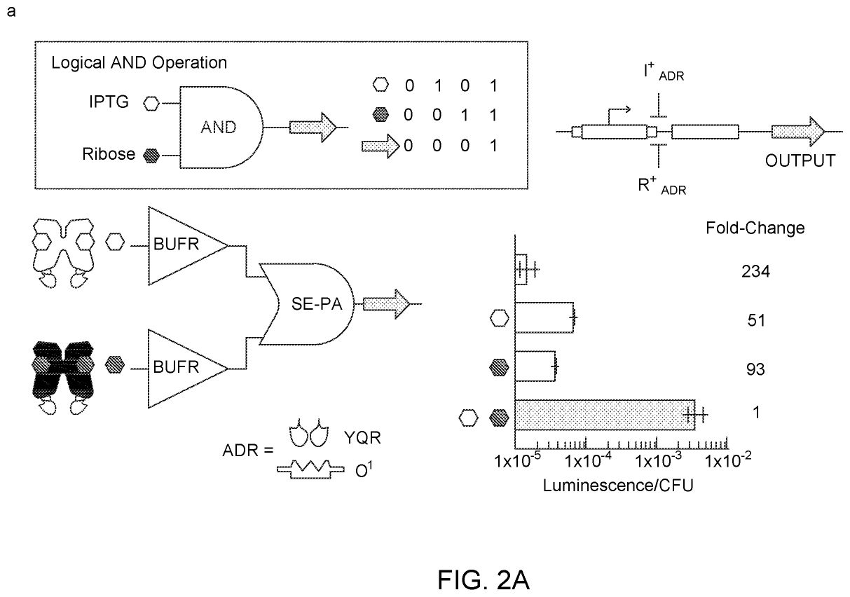

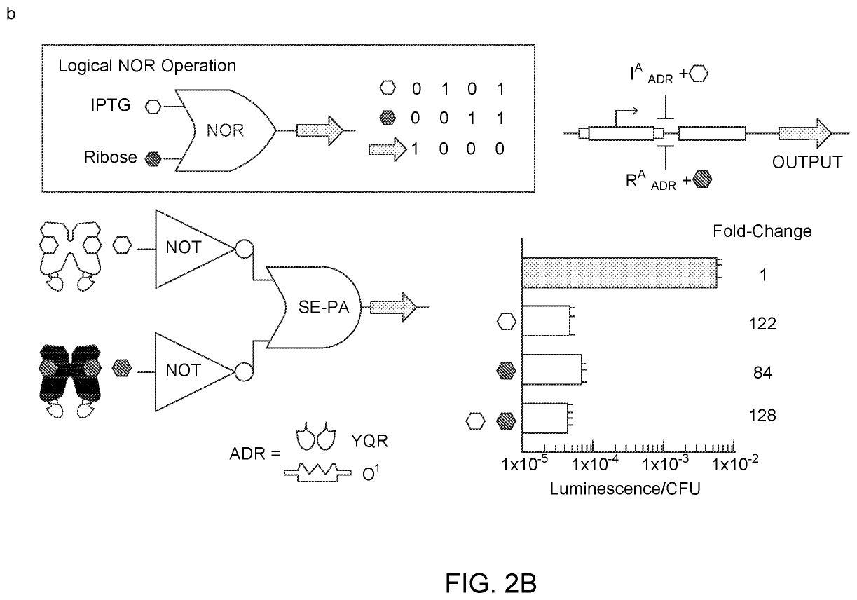

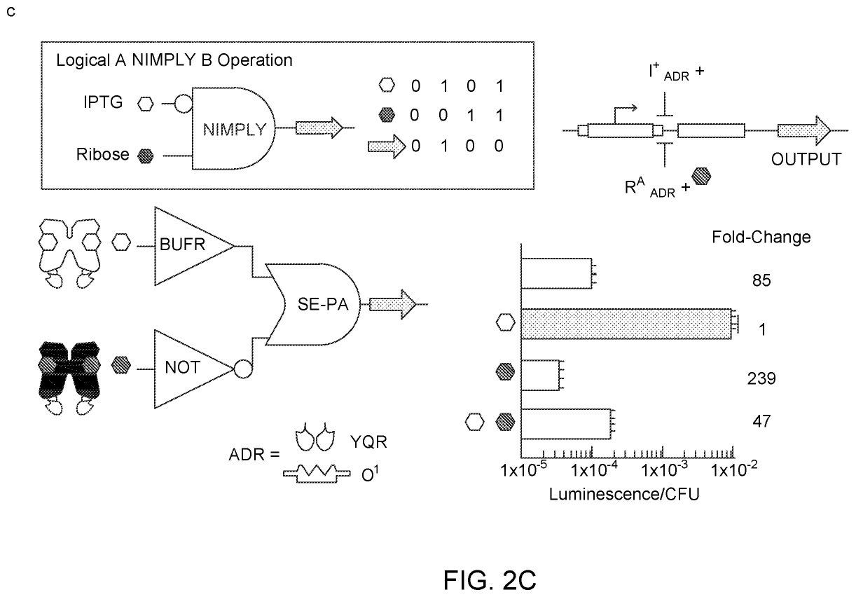

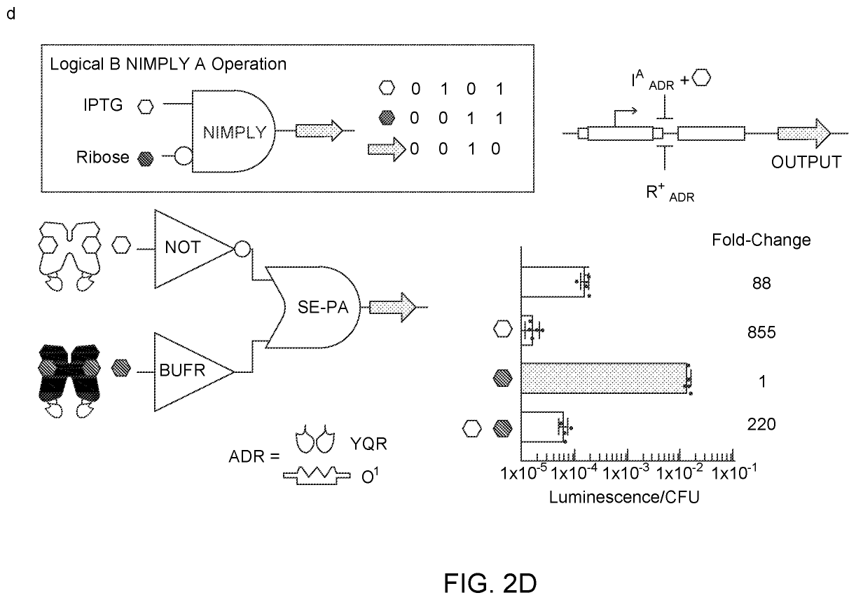

Constructing fundamental sets of two-input single-output logical operations in B. thetaiotaomicron. An important feature of the system of transcription factors is the ability to systematically pair two non-synonymous transcription factors via one regulated promoter (one layer) to construct fundamental two-input logical operations. In principle, using a single tandem operator-promoter genetic architecture four simple (one layer) two-input single-output combinational programs can be constructed—i.e., (i) AND, (ii) NOR, (iii) A NIMPLY B, and (iv) B NIMPLY A. To construct a two-input AND gate in the B. thetaiotaomicron chassis cell, two non-synonymous repressors I⁺_{YQR} and R⁺_{YQR} were paired (i.e., two BUFFER gates that were responsive to different input signals) and directed both transcription factors to a single cognate P_{O1} tandem operator-promoter, which regulated a luciferase output (FIG. 2A). The corresponding phenotype objectively resulted in an AND logical operation, where the circuit only allowed the production of luciferase when IPTG and D-ribose were both present. Next, an antithetical NOR gate was constructed using the same P_{O1} tandem operator-promoter (FIG. 2B). However, for the NOR gate I^{A(9)}_{YQR} with R^{A(1)}_{YQR} was paired, directing both anti-repressors (i.e., two non-synonymous NOT operations) to the same DNA regulatory element. The resulting two-input NOR logical operation functioned as expected—where the addition of IPTG or D-ribose resulted in the rejection of the luciferase output.

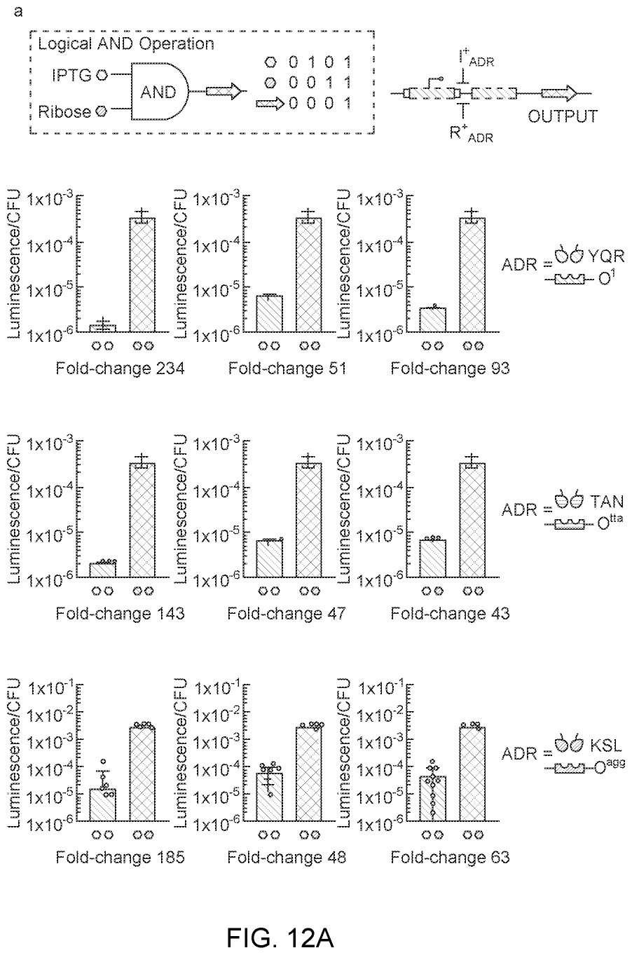

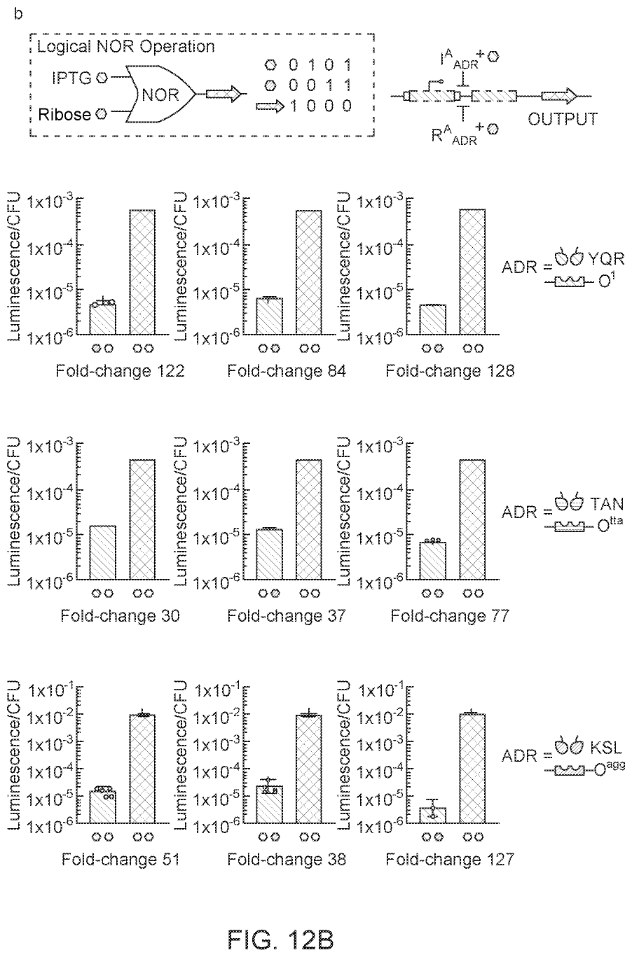

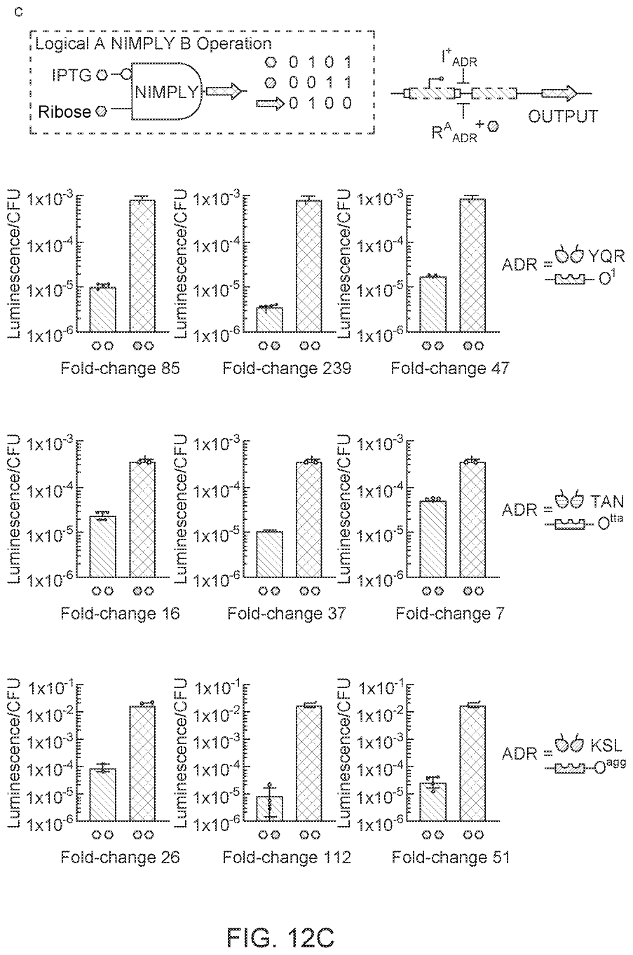

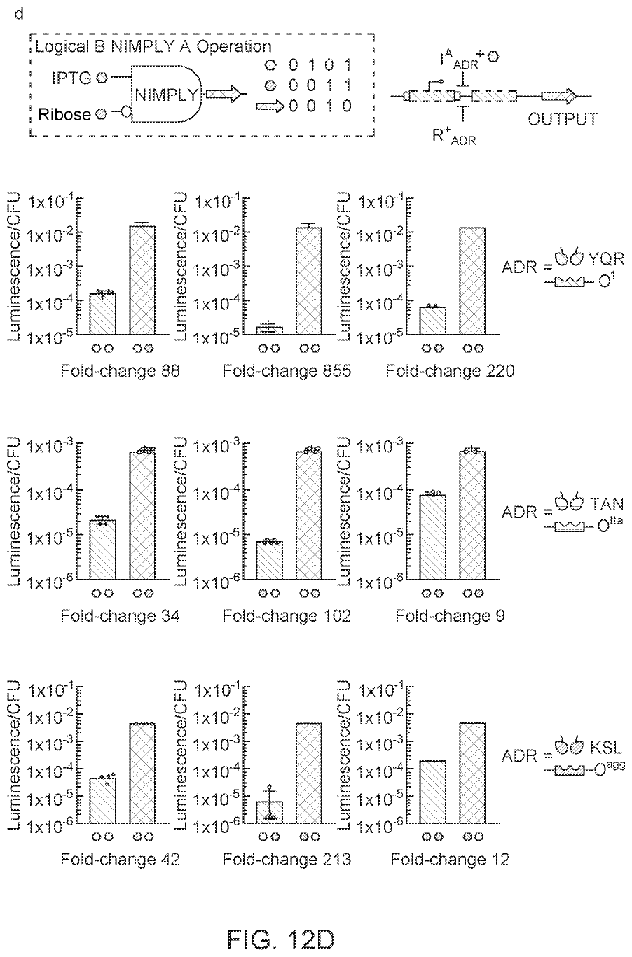

In addition, single-input BUFFER and NOT gates directed via the same P_{O1} tandem operator-promoter were mixed to form two-input NIMPLY logical operations. Namely, by pairing I⁺_{YQR} with R^{A(1)}_{YQR} an A NIMPLY B logical operation was generated (FIG. 2C). Likewise, the B NIMPLY A logical operation was obtained via the complementary set of transcription factors—i.e., I^{A(9)}_{YQR} with R⁺_{YQR} (FIG. 2D). To demonstrate that single-layer gate construction was generalizable when directed to different promoter elements, AND, NOR, A NIMPLY B, and B NIMPLY A were constructed via two additional tandem operator-promoters—Pₜₜₐ, and P_{agg}—which are cognate to the TAN and KSL DNA binding domains of a given transcription factor, respectively, but orthogonal to one another (FIG. 12). Qualitatively, all logical operations constructed from the transcription factors with alternate DNA binding functions resulted in the same objective phenotypes observed for sets directed to the P_{O1} tandem operator-promoter. However, quantitatively the dynamic range was variable, and it was contemplated that this was due to variation in promoter strength, in addition to any differences in the inherent protein-DNA interactions.

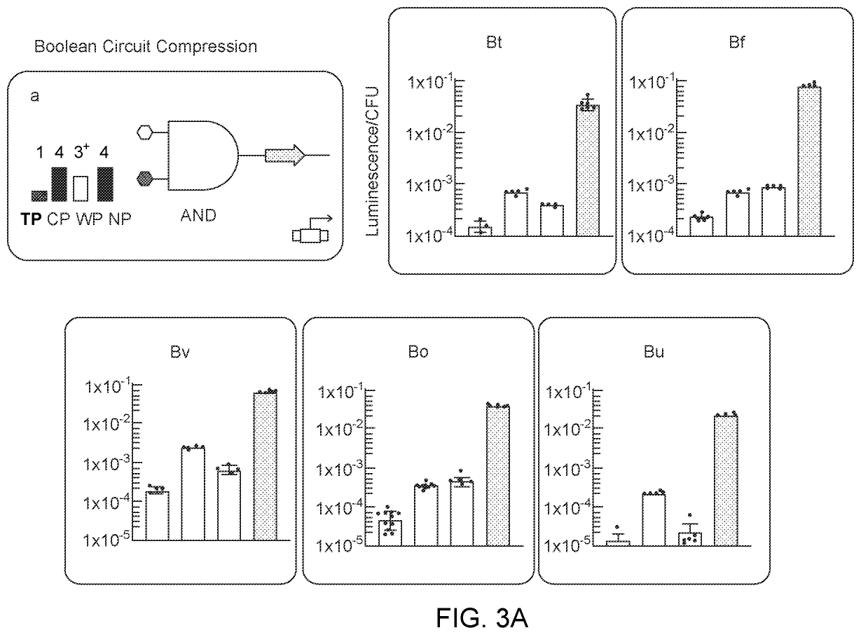

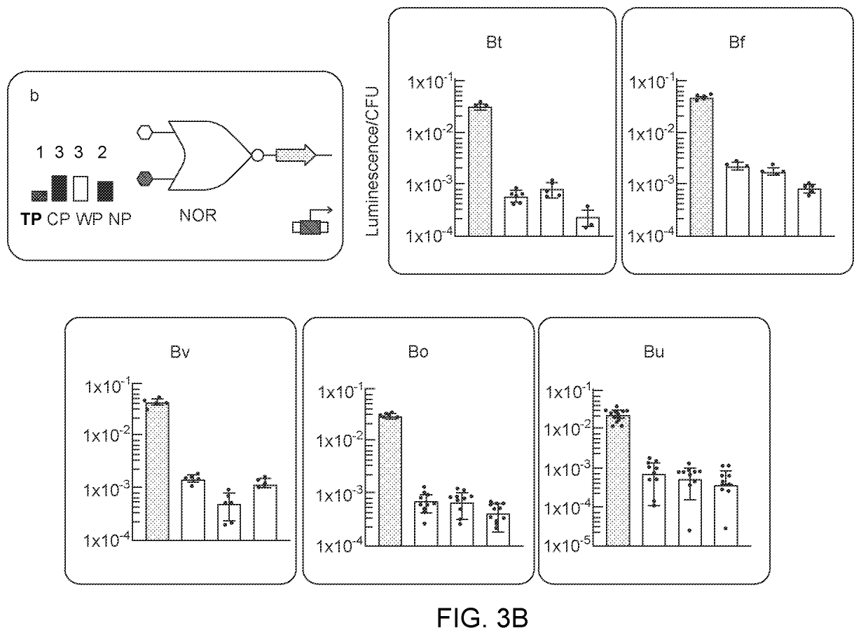

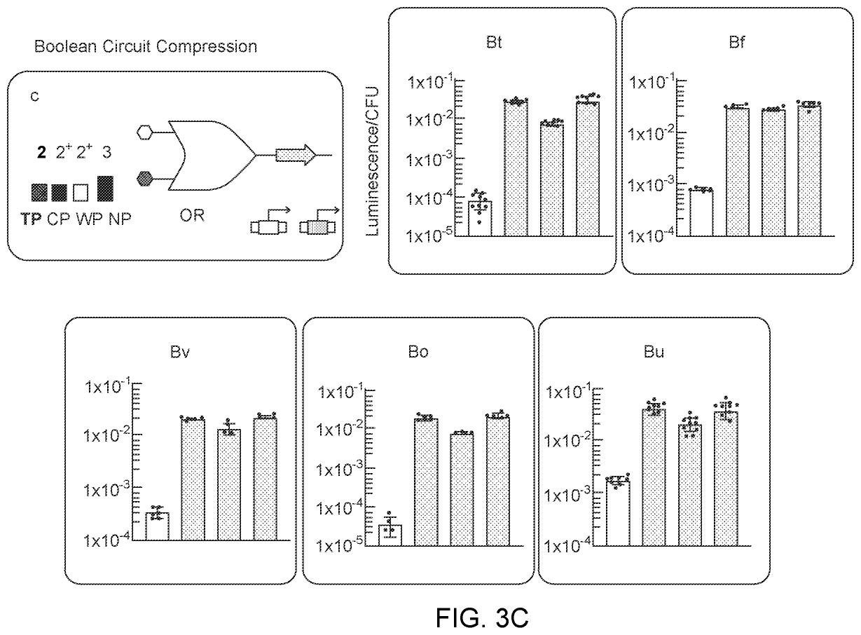

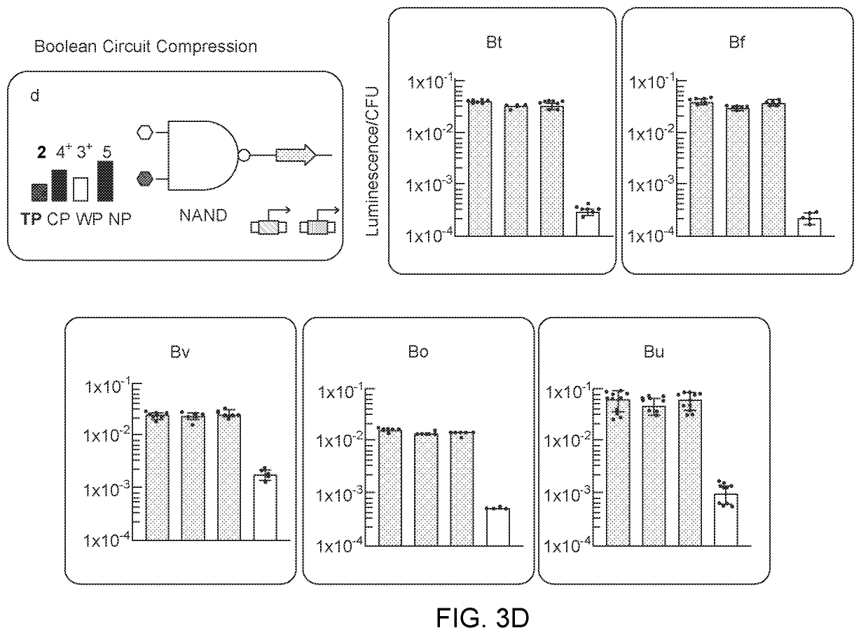

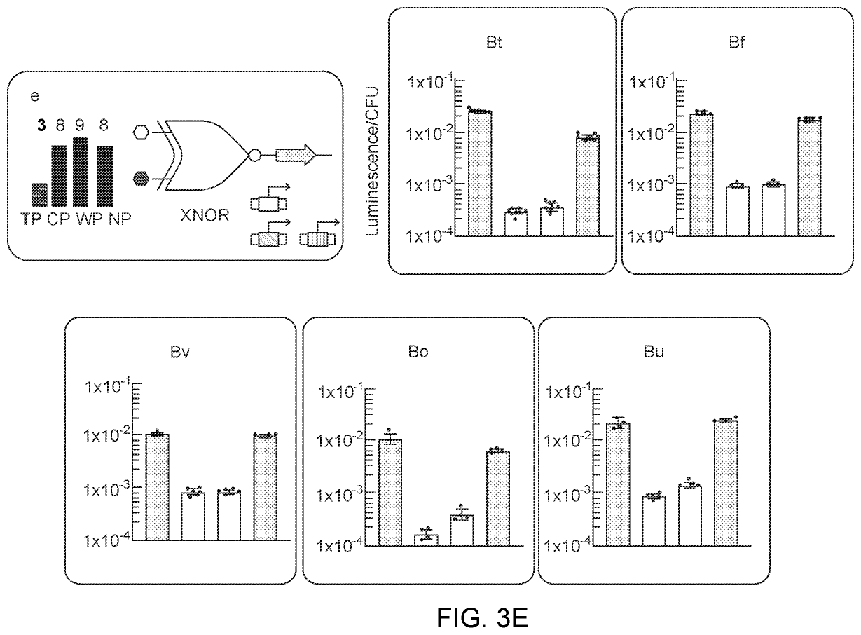

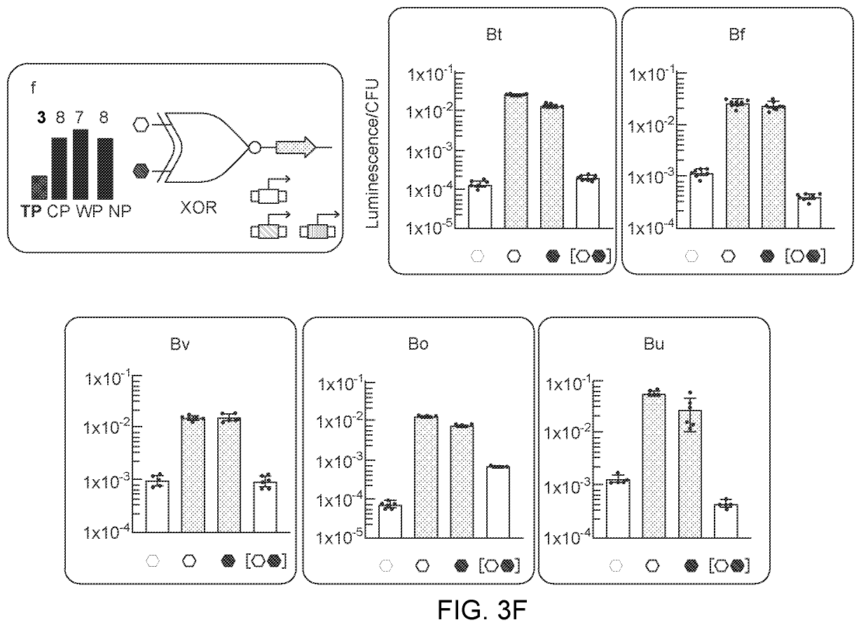

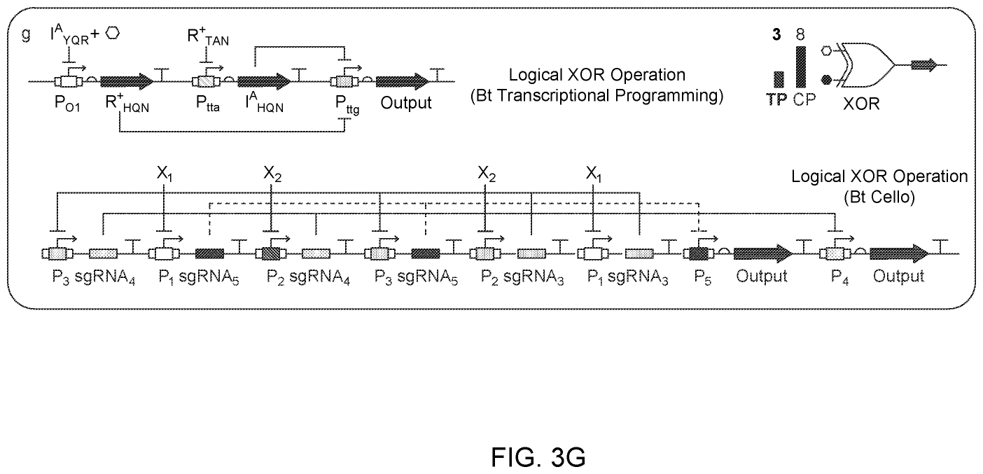

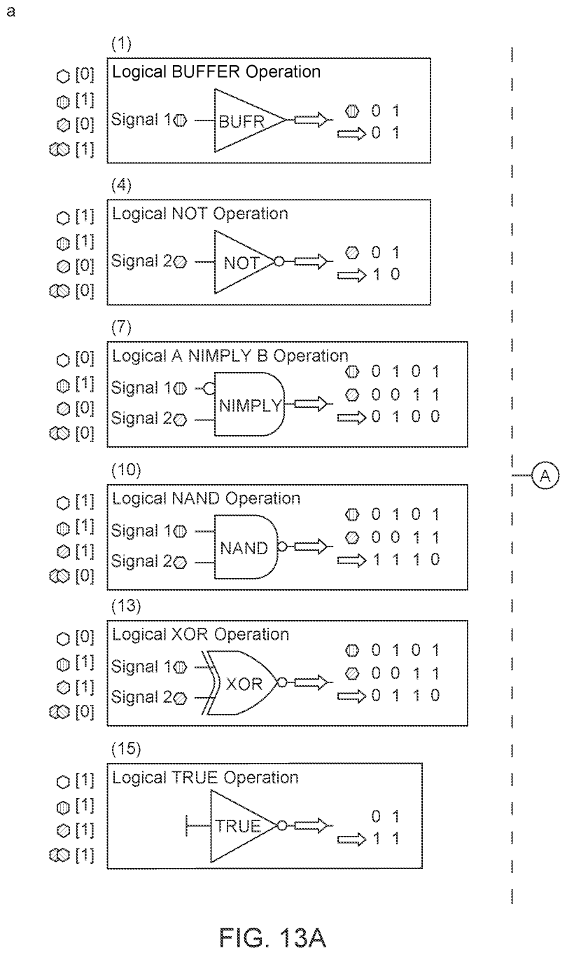

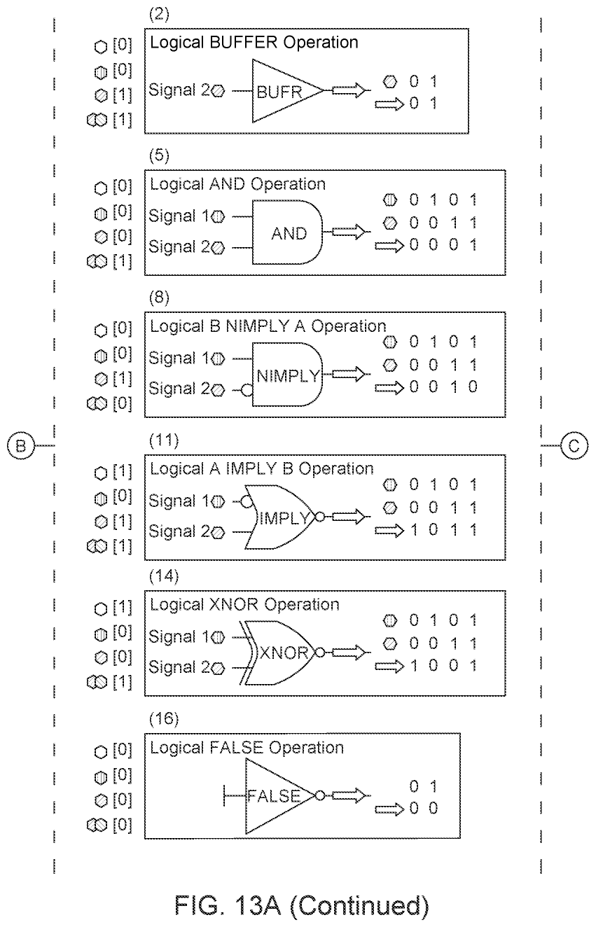

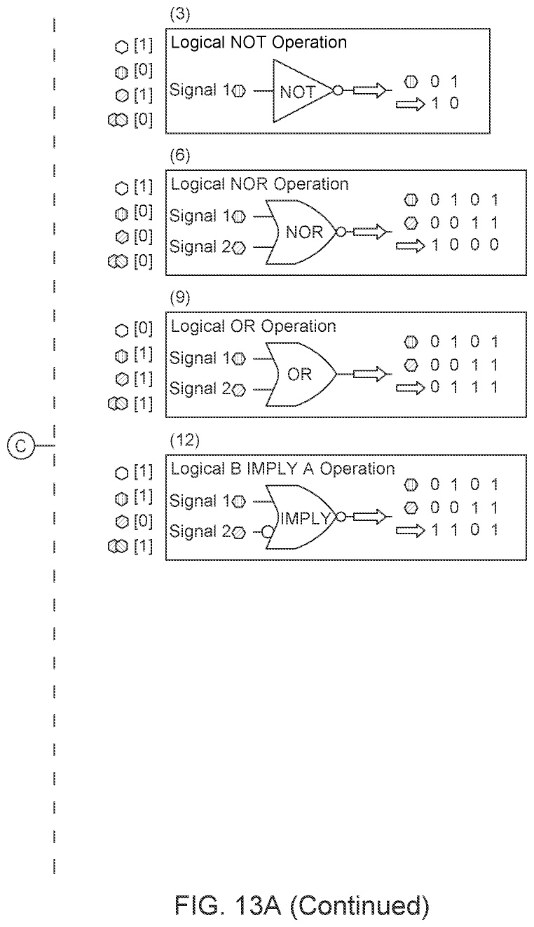

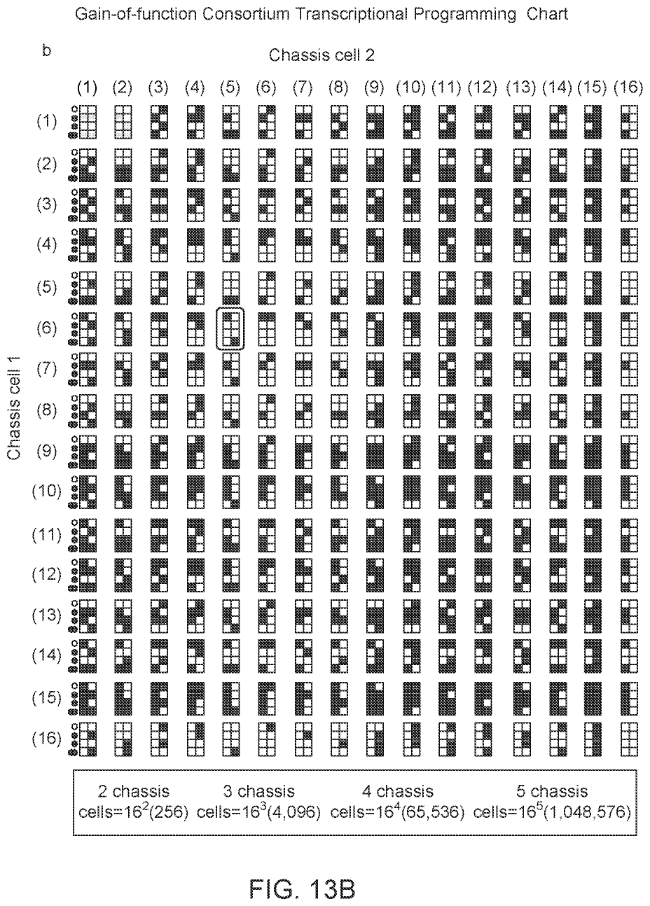

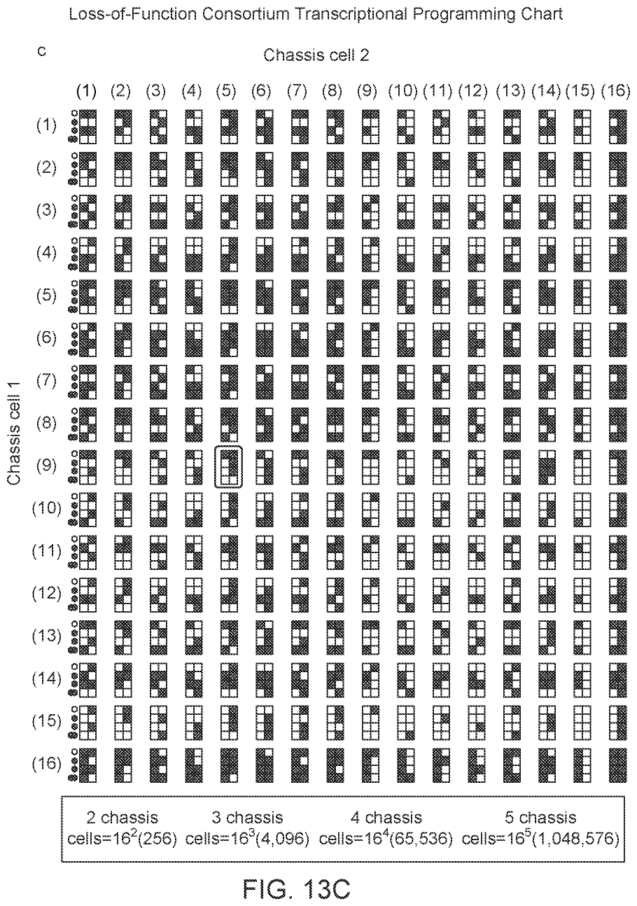

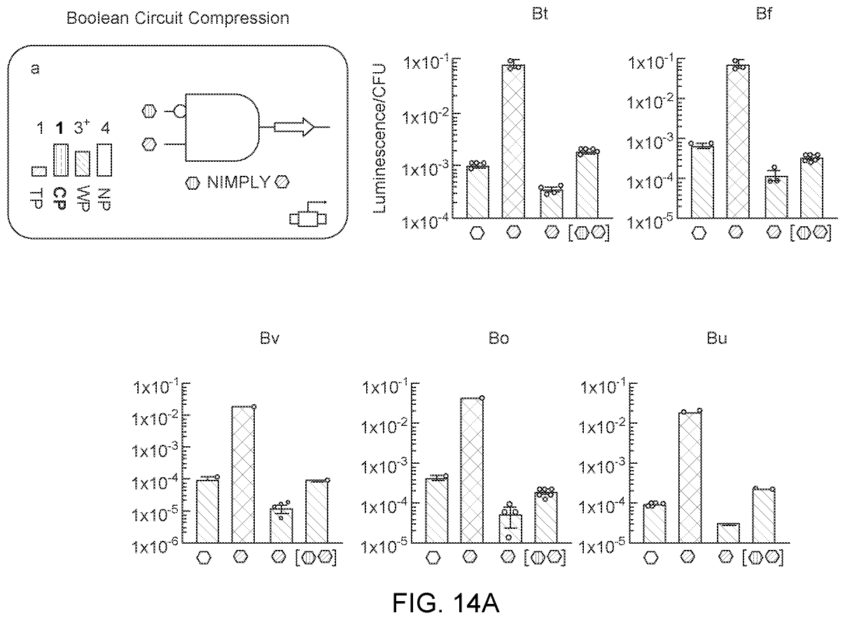

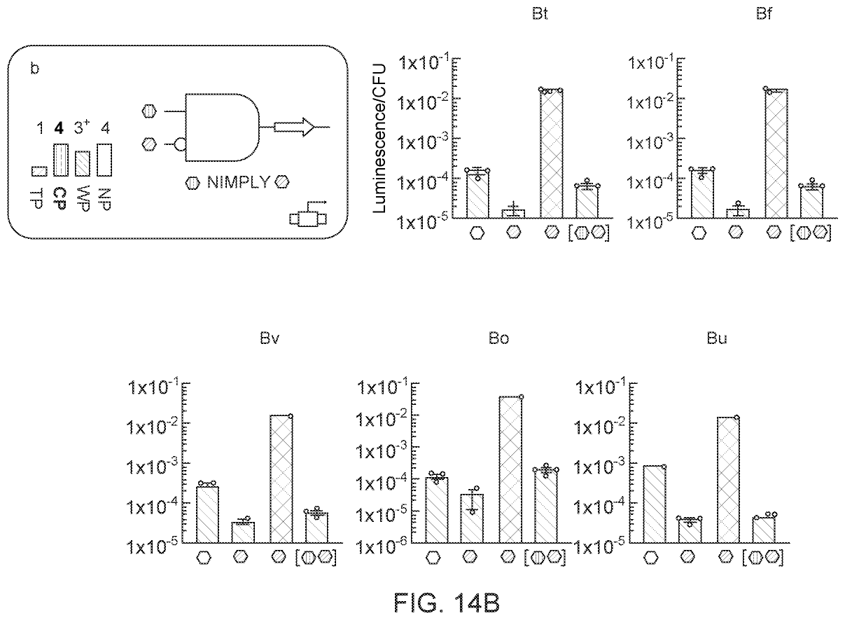

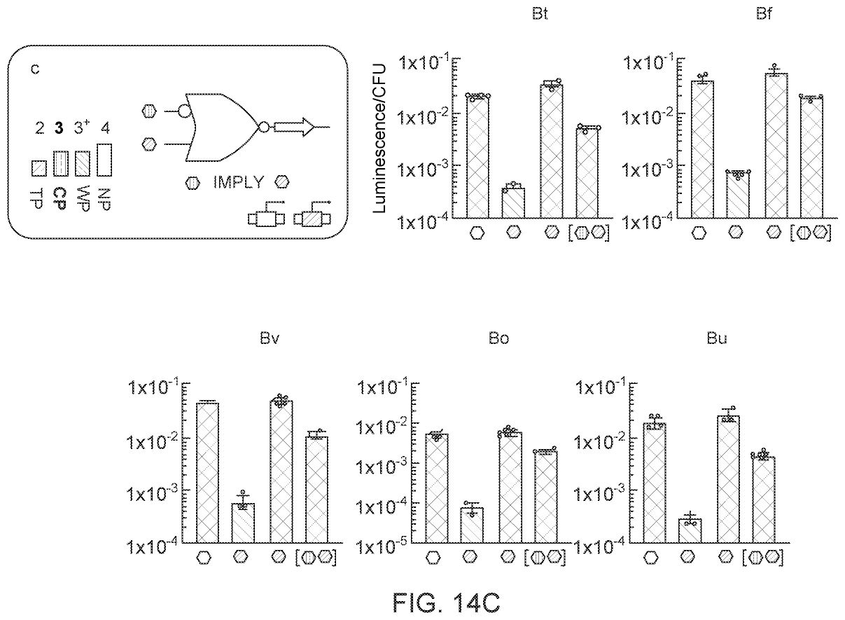

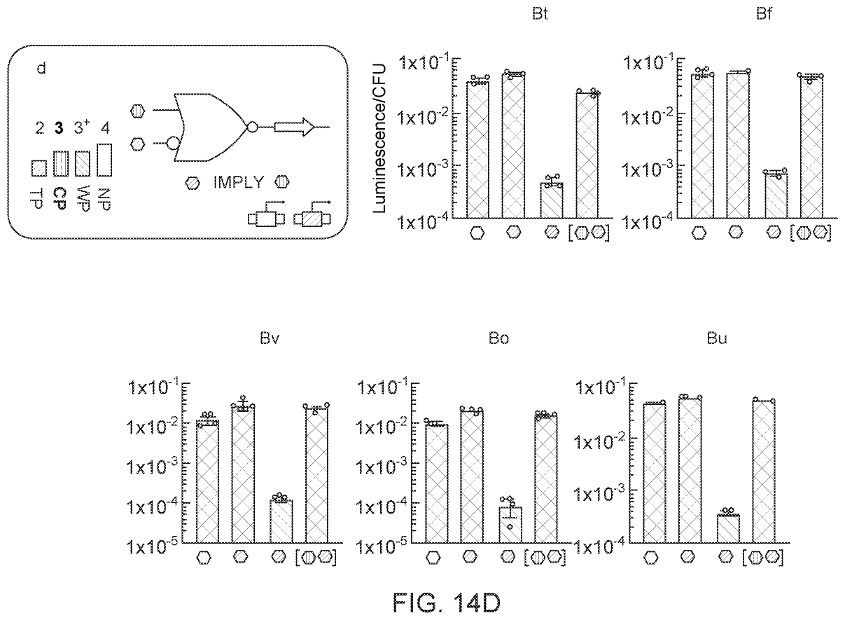

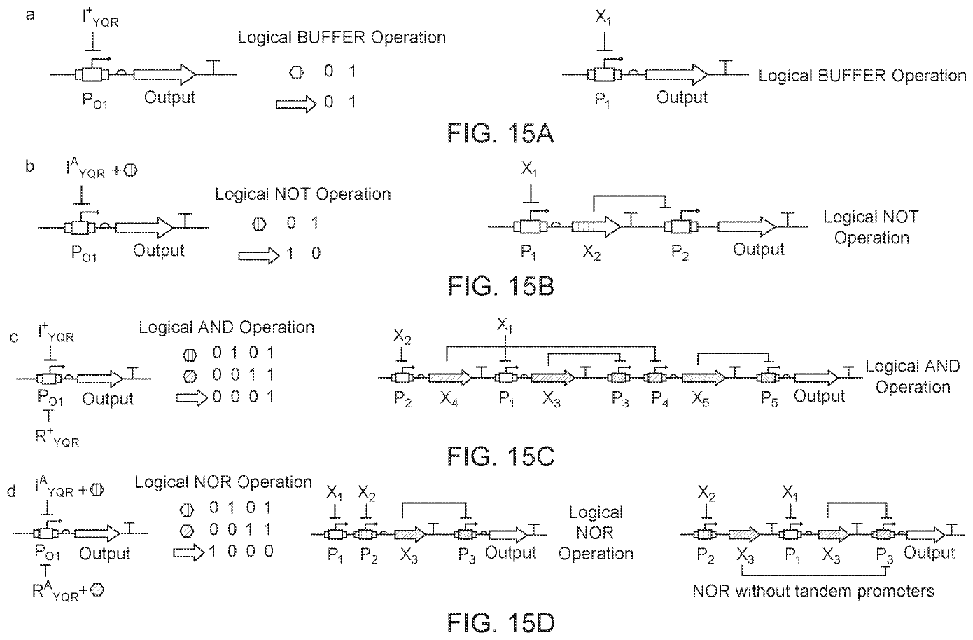

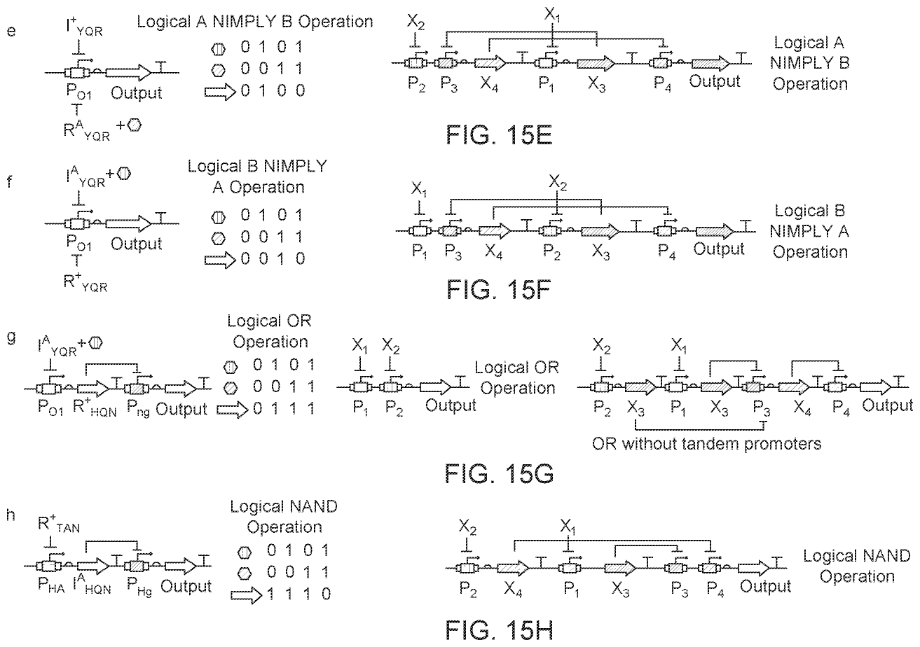

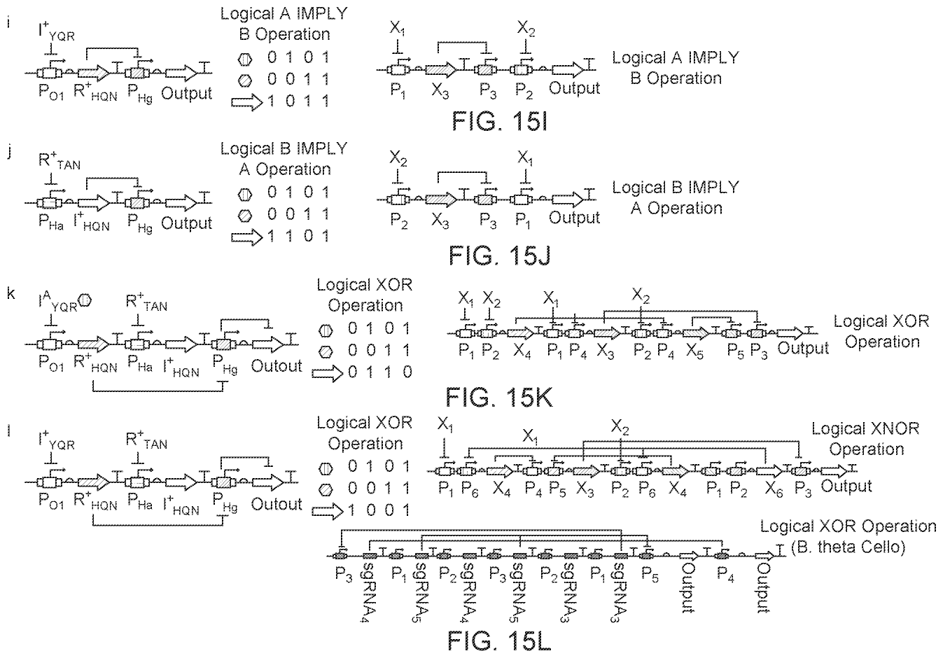

Combinational (feedforward) programming in B. thetaiotaomicron and circuit compression. In principle, given: (i) 2 non-synonymous repressors, (ii) 2 antithetical anti-repressors, (iii) 3 orthogonal operator-promoters, and (iv) the ability to feedforward information—all 16 Boolean logic gates can be systematically constructed via transcriptional programming (FIG. 13A). An important feature of this programming structure is that the (or compress) gate construction can be constructed via coupled anti-repression—even in the context of feedforward processing—resulting in the reduction of the endogenous resources required for operation. The gate compression is defined in terms of the number of inducible promoters for a given logical operation, relative to similar logic gates constructed in other biotic systems—see Example 2. To test these assertions, all remaining two-input gates that required the use of feedforward processing in the B. thetaiotaomicron chassis cell—namely, OR, NAND, A IMPLY B, B IMPLY A, XOR, and XNOR (see FIG. 3 and FIG. 14) were built, designed, and tested. The rational design and construction of the remaining feedforward gates was informed by the performances of the individual transcription factors (see FIG. 9 and Example 2). The design-build-test cycles were initiated focusing on the development of simple two-layer feedforward logical operations in which the following was paired: (i) a single-input single-output logical operation with the output defined as a single non-synonymous transcription factor, with (ii) a second layer that contained a regulatable promoter and luciferase output. Using this general workflow, OR, NAND, (FIGS. 3C, 3D and FIGS. 15G and 15H) was constructed and both IMPLY logical operations (FIGS. 14C, 14D and FIGS. 15I, 15H, and 15J). Next, complex feedforward logical operations composed of three layers were constructed and tested in which (i) 2 single-input single-output circuits (where the output for each primary layer was defined as a non-synonymous transcription factor) operated in parallel, relative to (ii) a second layer composed of a regulatable promoter (upstream of a luciferase output) with the capacity to direct and couple the transcription factors from the previous layer. This workflow allowed for construction of two of the most complex logic gates from a Boolean perspective—XNOR and XOR (FIGS. 3E, 3F, and FIGS. 15K and 15L). Qualitatively, all representative two-input feedforward gates resulted in the correct input-output phenotype, with dynamic ranges greater than 50.

To illustrate circuit compression, a relative comparison of transcriptionally programmed circuits was conducted to Cello circuits (the state-of-the-art in gene circuit design), a chemical wires approach that utilized multiple chassis cells, and general Boolean NOR layering (logical axiom), (FIG. 3 and FIG. 15). The relative comparison was achieved by way of counting the number of regulated promoters used to construct a given circuit—also see Example 2. However, in the case of abstract Boolean NOR layering an input node was regarded as a promoter equivalent. In every two-input case, transcriptional programming achieved significant circuit compression over chemical wires and general Boolean NOR layering. While the chemical wires and Boolean approaches provide a broader context for the evaluation of circuit compression, the most meaningful relative comparison was to the Cello genetic circuits—as this technology contains a given circuit to a single chassis cell, akin to transcriptional programming (FIGS. 3, 14, and 15). In all cases (with the exception of BUFFER and OR) transcriptional programming resulted in significant circuit compression—i.e., required significantly fewer promoters. In the case of the BUFFER and OR gates, transcriptional programming was on par with Cello—requiring one and two promoters, respectively. Notably, the OR gate is a unique (ad hoc) Cello construct composed of two tandem promoters. In other words, if the OR gate was constructed via inversion alone it would require a minimum of four promoters (FIG. 15G and Example 2). To illustrate circuit compression, the XNOR and XOR gates can be used as exemplars of the extent of gate compression that can be achieved via transcriptional programming—as these are the two most complex logical operations developed from a NOR programming perspective. In both cases approximately a three-fold decrease in circuit complexity (promoter requirements) was achieved via transcriptional programming—relative to Cello circuit designs. Notably, the XOR logical operation represents the most direct comparison between Cello and transcriptional programming in the same chassis cell—herein both gates have been constructed in B. thetaiotaomicron. Namely, the Cello XOR gate was composed of 8 regulated promoters and two output genes while the XOR gate constructed via transcriptional programming only required 3 promoters and one output gene (FIG. 3G).

Transferring transcriptional programming to human donor Bacteroides chassis cells. Once transcriptional programming in B. thetaiotaomicron was established, it was contemplated that the programming edifice extended to other Bacteroides. Accordingly, all single-input (BUFFER and NOT) logical operations were tested in four additional Bacteroides species that are commonly found in humans—i.e., B. fragilis, B. ovatus, B. uniformis, and B. vulgatus (FIG. 1). Namely, 10 non-synonymous BUFFER gates composed of the 5 I⁺ repressors and 5 R⁺ repressors and 5 cognate and orthogonal operator-promoters were tested—i.e., the same set developed in B. thetaiotaomicron (see FIGS. 1B, 1C, and FIG. 9). Qualitatively, all 10 BUFFER gates were functional in B. fragilis, B. ovatus, B. uniformis, and B. vulgatus. Moreover, the general performance of each BUFFER operation was similar between Bacteroides strains and comparable to the performances and trends observed with variation in DNA-binding function in B. thetaiotaomicron. Next, each of the antithetical NOT unit operations was tested in B. fragilis, B. ovatus, B. uniformis, and B. vulgatus. In this experiment, the individual performances of the corresponding 5 I^{A(9)} and 5 R^{A(1)} anti-repressors were tested using the same 5 cognate operator-promoters (see FIGS. 1D, 1E, and FIG. 9). Congruent with previous observations, the NOT unit operations had comparable performances to those observed in B. thetaiotaomicron.

Given that both single-input logical operations (BUFFER and NOT) functioned in B. fragilis, B. ovatus, B. uniformis, and B. vulgatus, it was contemplated that the corresponding single-layer two-input logical operations—(i) AND, (ii) NOR, (iii) A NIMPLY B, and (iv) B NIMPLY A—could be constructed via the same circuit design rules used in the B. thetaiotaomicron chassis cell (FIGS. 15C, 15D, 15E, and 15F). Accordingly, the archetypal AND gate (i.e., pairing I⁺_{YQR} and R⁺_{YQR} with the P_{O1} operator-promoter) was transferred into each of the four representative Bacteroides (see FIG. 3A). Constraining the composition of the AND program allowed for conduction of a relative comparison between representative chassis cells to assess to what extent the transcriptional program was impacted by the genetic and metabolic differences between Bacteroides strains. Qualitatively, all AND gates resulted in the correct truth table—i.e., all requiring two inputs to induce the expression of luciferase. Quantitatively, all AND gates displayed large dynamic ranges (greater than 200) when comparing zero-input expression to two-input expression. The AND gate in B. uniformis was the best performing logical operation followed by B. ovatus, with dynamic ranges of 1468 and 781, respectively. The AND gates in B. fragilis and B. vulgatus were nearly on par as logical operations, with dynamic ranges of 325 and 310, respectively (under the same conditions).

Next, an antithetical NOR gate (i.e., pairing I^{A(9)}_{KSL} and R^{A(1)}_{KSL} with the P_{agg} operator-promoter) was tested in B. fragilis, B. ovatus, B. uniformis, and B. vulgatus (FIG. 3B). Each representative NOR gate resulted in the same qualitative outcome—i.e., rejecting the output in the presence of one or both input signal(s) (IPTG and D-ribose). All NOR gates had greater than 50-fold dynamic range for zero inputs (ON-state) relative to both inputs (OFF-state), with the exception of B. vulgatus which had a dynamic range of 35—which was consistent with single input logical operation performances. Congruent with the aforementioned single-layer two-input logical operations, A NIMPLY B (FIG. 14A), and B NIMPLY A (FIG. 14B) were functional across all representative Bacteroides chassis cells.

Once all fundamental single-layer logical operations were demonstrated as functional in the four representative human donor Bacteroides, the remaining two-input feedforward logic gates were tested—i.e., OR, NAND, XNOR, XOR, A IMPLY B, B IMPLY A (see FIGS. 3C, 3D, 3E, 3F, and FIGS. 145C and 14D). Here the same circuit designs was used as outlined in FIGS. 15G, 15H, 15I, 15J, 15K, and 15L. In general, all compressed two-input logic gates functioned in B. fragilis, B. ovatus, B. uniformis, and B. vulgatus—and objectively resulted in truth tables. No apparent trends emerged between chassis cells, and the moderate differences in performance between logic gates in a given chassis cell were interpreted as the result of differences in metabolic potential inherent to a given Bacteroides strain. Collectively, these results demonstrated that the compressed logic gates could generally be imbued broadly across a panoply of Bacteroides chassis cells—representing a robust tool for programmable gain-of-function that can be employed within a consortium.

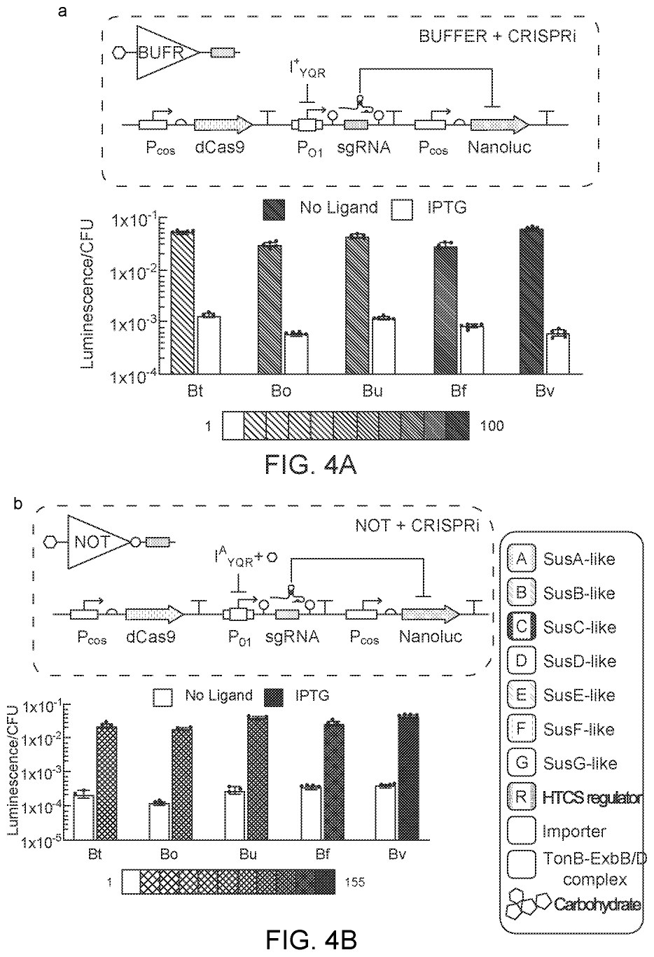

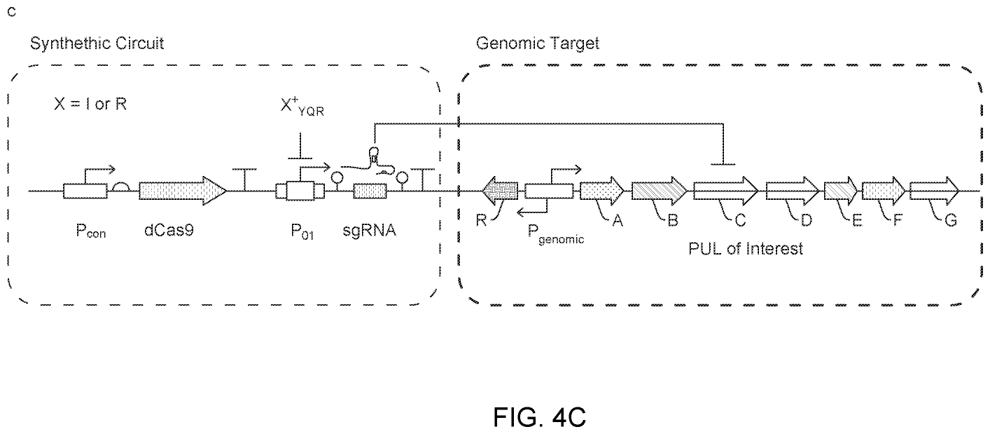

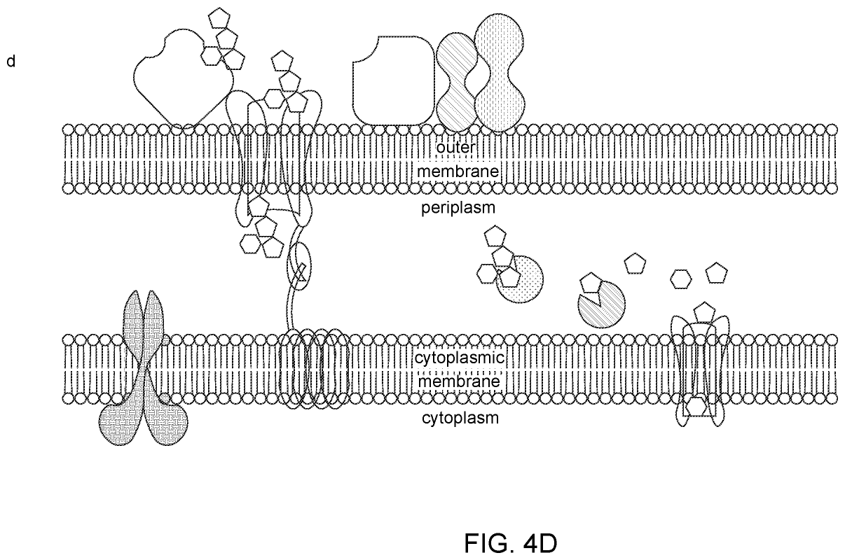

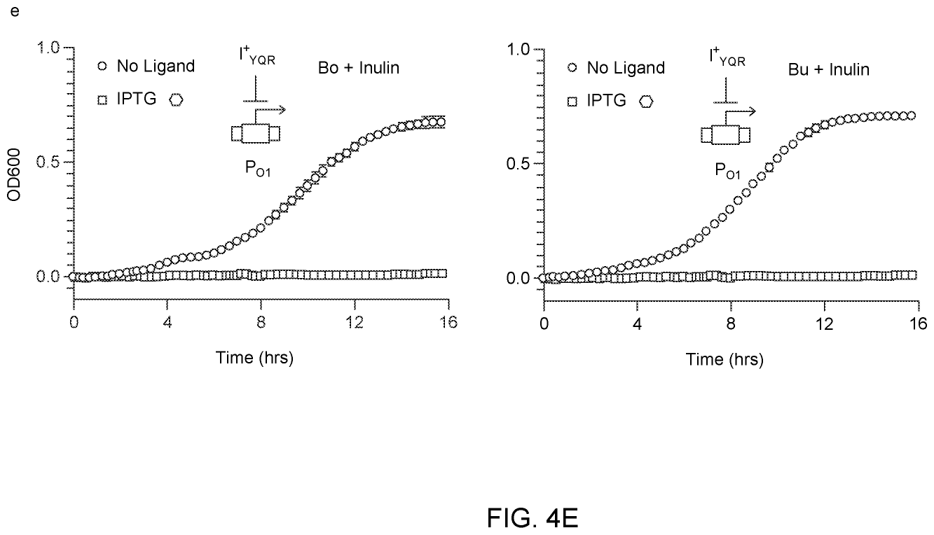

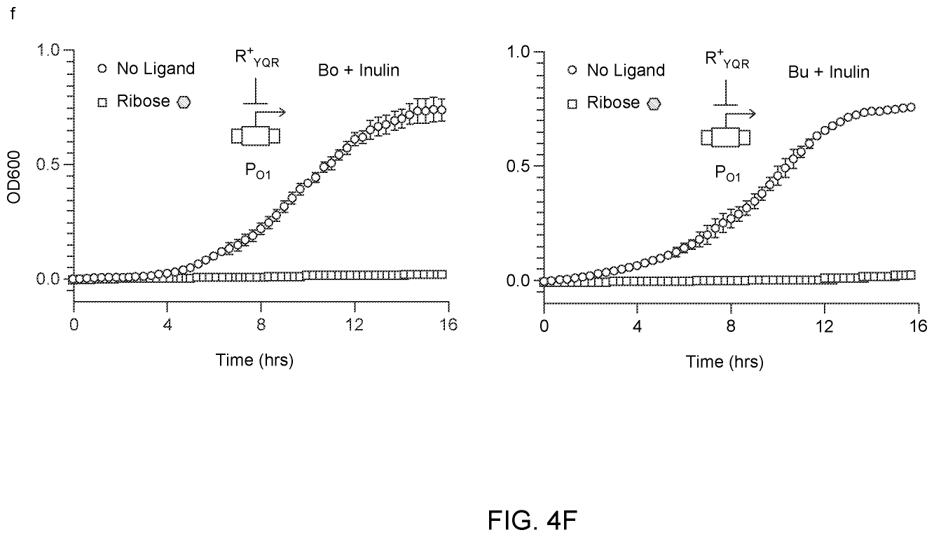

Transcriptional programming paired with CRISPR interference in Bacteroides chassis cells. Given the high regulatory performance observed in the logic circuits (both simple and combinational) with inert luciferase outputs, it was contemplated that transcriptional programming could be effectively paired with CRISPR interference (CRISPRi) technology—in each of the representative Bacteroides chassis cells. To test this assertion, iterations of regulated single guide RNA (sgRNA) were first built, designed, and tested that targeted the NanoLuc reading frame in B. thetaiotaomicron (FIG. 4A). Briefly, a BUFFER gate (I⁺_{YQR} with cognate operator-promoter P_{O1}) was developed that regulated the production of the sgRNA transcript aimed at knocking down NanoLuc expression upon induction with IPTG. The P_{cfxA} promoter with tandem core and proximal operators was modified to allow for the production of functional sgRNAs. A hammerhead (HH) ribozyme was fused 4 bp downstream of the proximal operator, followed by the 102 bp sgRNA scaffold and a hepatitis delta virus (HDV) ribozyme. The inclusion of the ribozymes ensured that the minimal sgRNA was produced following transcription, cleaving extraneous 5′ and 3′ sequences. For all CRISPRi experiments, dCas9 was constitutively expressed from the P₁ promoter and no modification was made to the expression levels of transcription factor regulators. Congruent with the design goal, induction of the BUFFER gate resulted in a ˜40-fold reduction of observed NanoLuc production—thus demonstrating that the system is an effective tool for knocking down a given gene in B. thetaiotaomicron. It was contemplated that this circuit would be functional in the four additional Bacteroides species, given the universal results of the transcriptional programming circuits demonstrated in the aforementioned results FIGS. 1 and 3). To test this assertion, the said synthetic circuit was introduced into the remaining Bacteroides chassis cells and measured the inducible NanoLuc knockdown performances (FIG. 4A). In all cases, each of the engineered chassis cells performed the inducible knockdown of the heterologous luciferase on par or better than that observed in the B. thetaiotaomicron chassis cell.

Next, the antithetical NOT gate paired with a CRISPRi genetic circuit was constructed in the B. thetaiotaomicron chassis cell. Here, the basic NOT operation was executed by the I^{A(9)}_{YQR} anti-repressor and cognate operator-promoter P_{O1} (FIG. 4B). As anticipated, this simple synthetic circuit resulted in a reciprocal phenotype upon anti-induction via the introduction of the IPTG ligand with a 110-fold dynamic range. Likewise, the integration of this permissive maintenance-of-function circuit (i.e., with ligand) resulted in similar phenotypes in each of the disparate representative Bacteroides chassis cells—i.e., B. fragilis, B. ovatus, B. uniformis, and B. vulgatus (FIG. 4B).

As evidenced with previous results, the successful implementation of the I⁺_{YQR} (BUFFER gate) and I^{A(9)}_{YQR} (NOT gate) with the cognate operator-promoter P_{O1} in a given chassis cell is a strong indicator that the broader transcriptional programming structure can be paired with CRISPR technologies. Here the justification was based on the observation that nearly all remaining single-input and two-input logical operations have similar or better fundamental performances relative to the tested circuits. Moreover, given the results of the single-input systems it was contemplated that all additional single-input and two-input logical operations could be used to regulate the production of any sgRNA transcript. Accordingly, additional iterations of this tool were not tested; rather, this assertion was demonstrated via case studies in which carbon utilization was manipulated in Bacteroides.

Controlling carbon utilization in Bacteroides via single-input programming. Bacteroides possess the ability to degrade a large number of polysaccharides due to specialized gene clusters termed polysaccharide utilization loci (PULs), see FIG. 4D. Bacteroides species harbor many PULs, each of which contain genes involved in the recognition, import, and degradation of a specific class of polysaccharide. Notably, different species in this genus often possess different catabolic abilities and cannot all utilize the same carbon sources. It has been previously demonstrated that controlling dietary carbohydrate composition can allow for stable colonization of Bacteroides possessing the requisite PUL machinery. This shows that GI population dynamics can be directly manipulated via a combination of specialized diet and in situ activation of transcriptional programs linked to PUL expression.

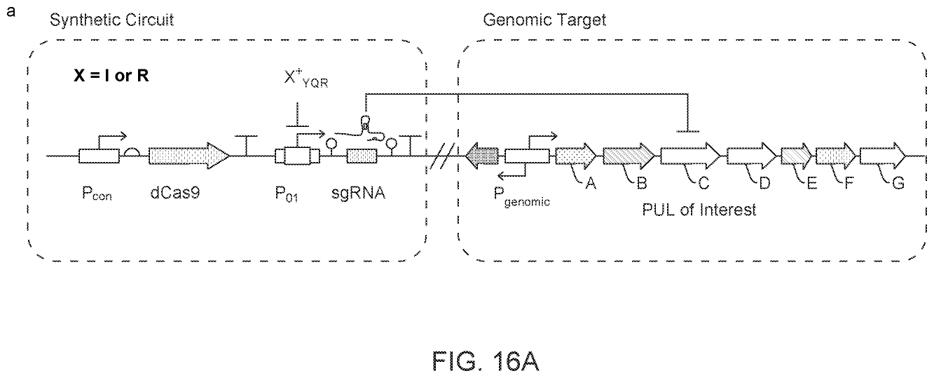

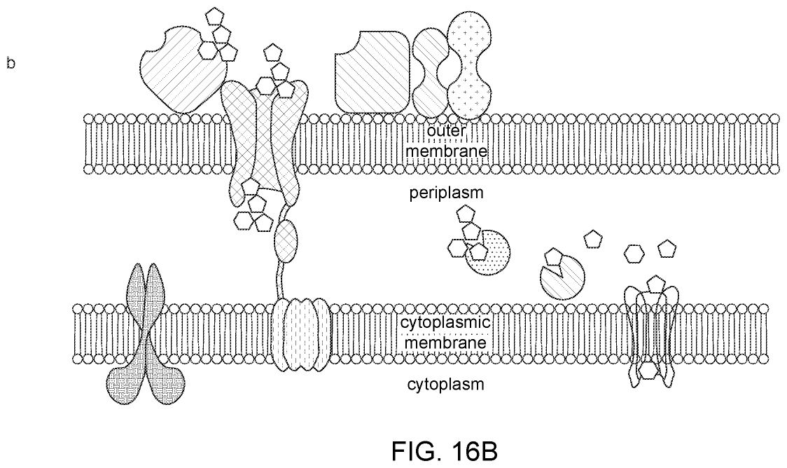

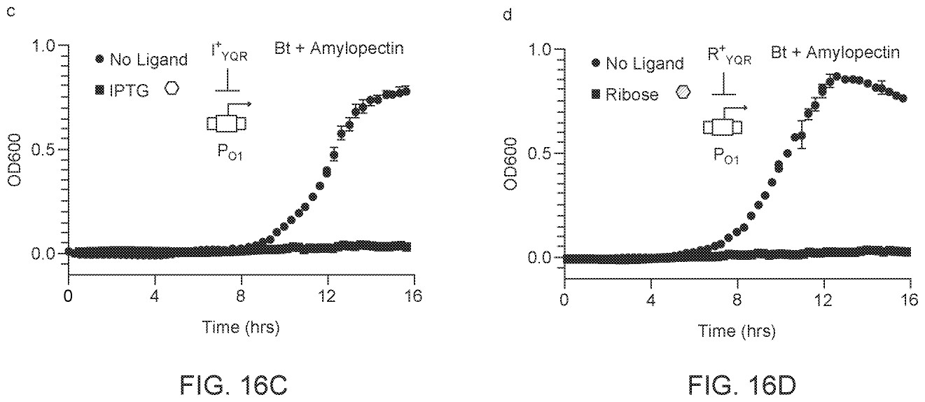

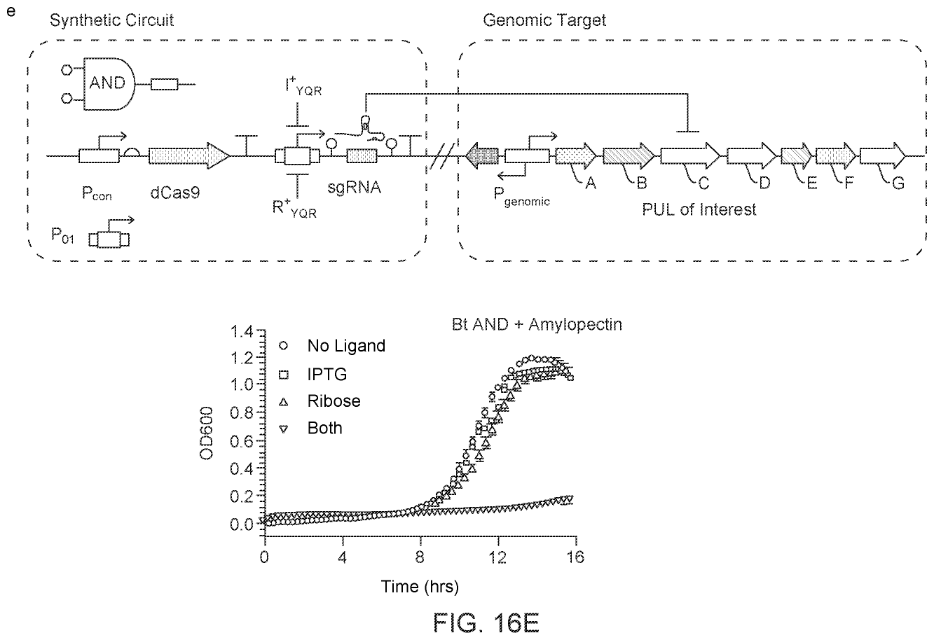

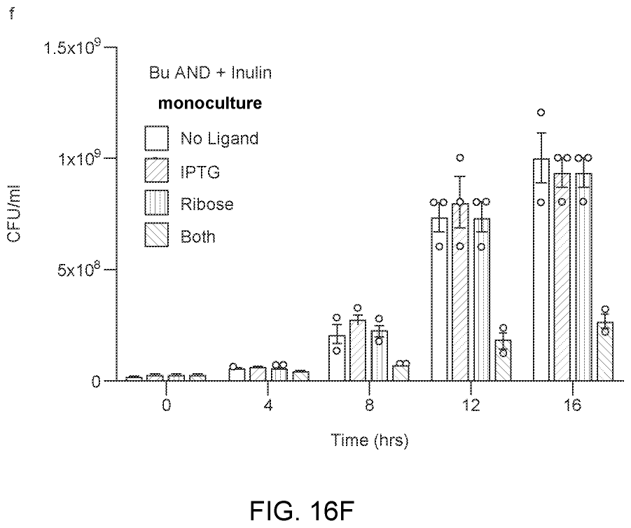

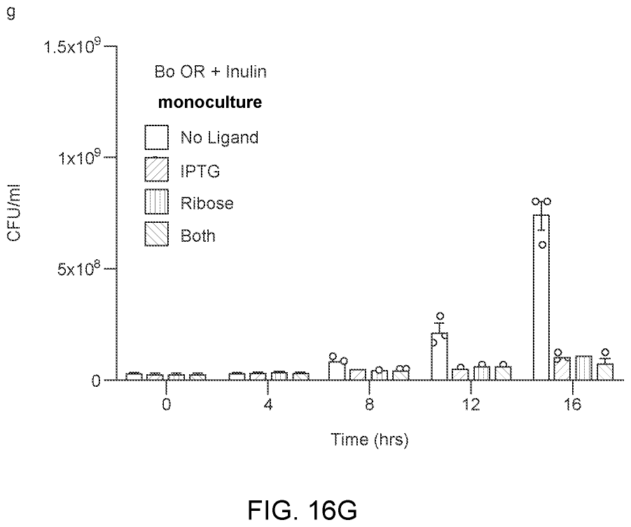

To demonstrate the utility of the programming edifice paired with CRISPRi, it was contemplated that sgRNAs could be designed to target SusC homologues implicated in the extracellular import of two relevant polysaccharides (inulin and amylopectin) for B. thetaiotaomicron, B. uniformis, and B. ovatus. The archetypal susC gene (starch utilization system gene C) in B. thetaiotaomicron is necessary for growth of this species on starch, and its homologues are highly conserved in Bacteroides PULs. The rationale for selecting these polysaccharides is that they represent two distinct classes of molecules implicated in GI microbiota homeostasis and are universally consumed by these three Bacteroides. Accomplishing this objective would enable control over population dynamics in the presence of a common (communal) carbon source. At the outset, simple monoculture experiments were conducted in which a LacI BUFFER operation was used to regulate the production of a sgRNA targeting SusC homologues in separate B. thetaiotaomicron, B. uniformis, and B. ovatus chassis cells as monocultures (FIGS. 4B and 4D). In B. uniformis and B. ovatus, the SusC homologue implicated in the uptake of inulin was targeted, whereas in B. thetaiotaomicron the SusC gene involved in the uptake of amylopectin was targeted. Using a simple synthetic circuit composed of an I⁺_{YQR} repressor and cognate regulated promoter P_{O1} (BUFFER gate), all three chassis cells resulted in the strong knockdown of the given PUL upon the addition of IPTG (FIG. 4E and FIGS. 16A, 16B, and 16C). This was observed as a loss of fitness for each monoculture in which a single carbohydrate (i.e., inulin or amylopectin) was present in the defined minimal media. Next, an analogous BUFFER CRISPRi synthetic circuit replacing the I⁺_{YQR} regulator with the R⁺_{YQR} repressor targeted at regulating the production of the same sgRNAs was built and tested. Congruent with the previous synthetic circuit, the introduction of the input signal D-ribose (in the corresponding defined minimal media) resulted in a loss of fitness in all three Bacteroides chassis cells in monoculture (see FIG. 4F and FIG. 16D

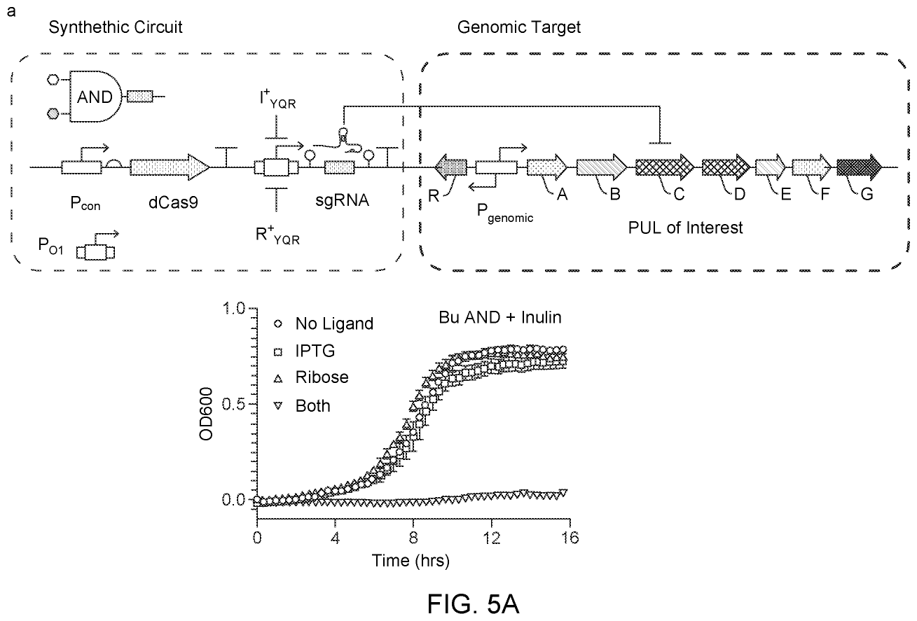

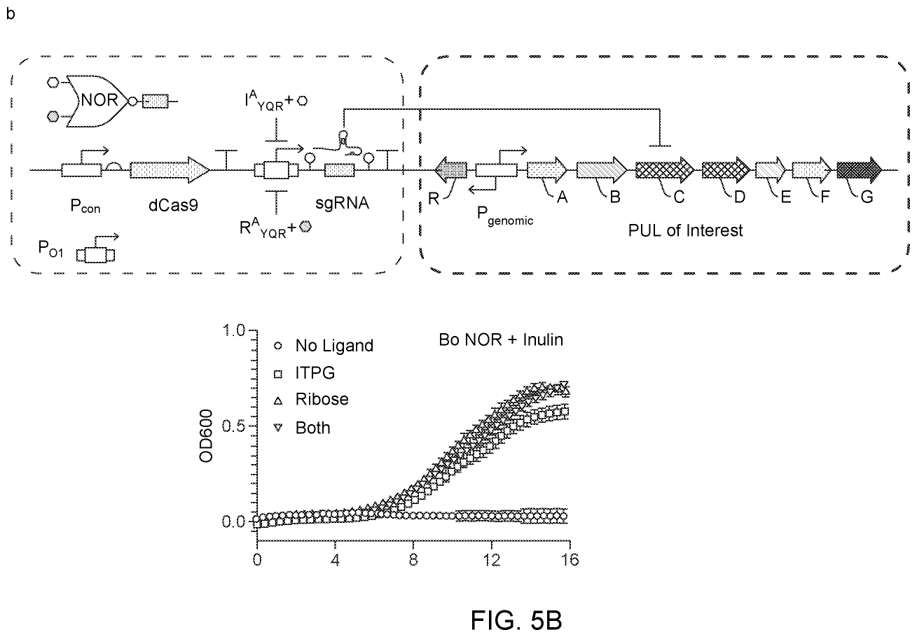

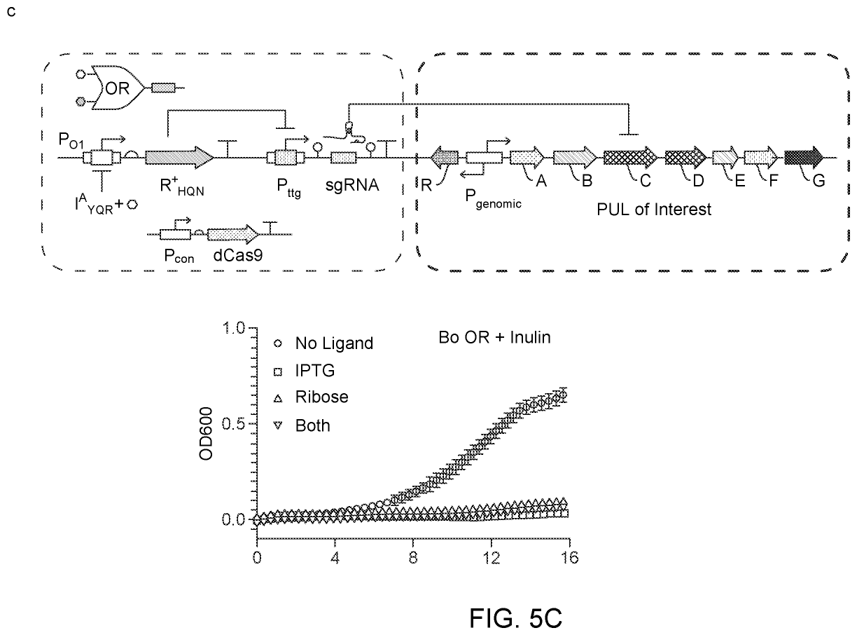

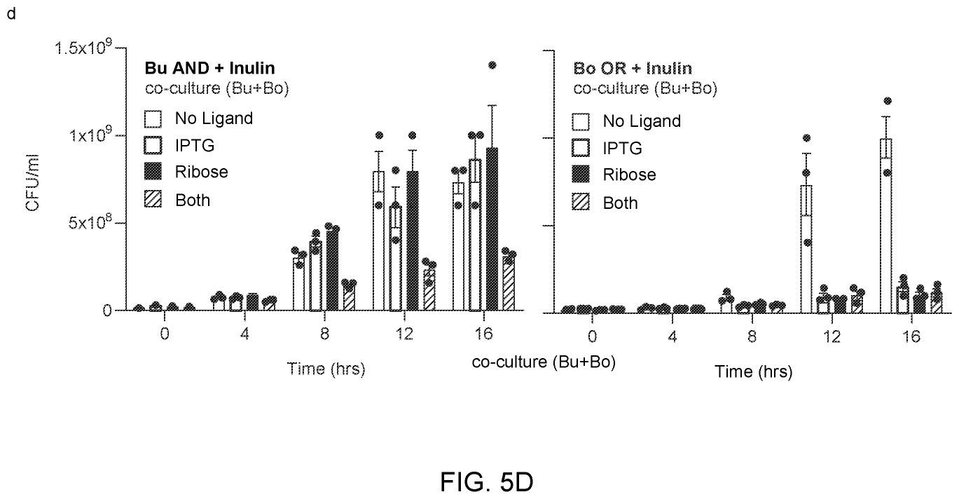

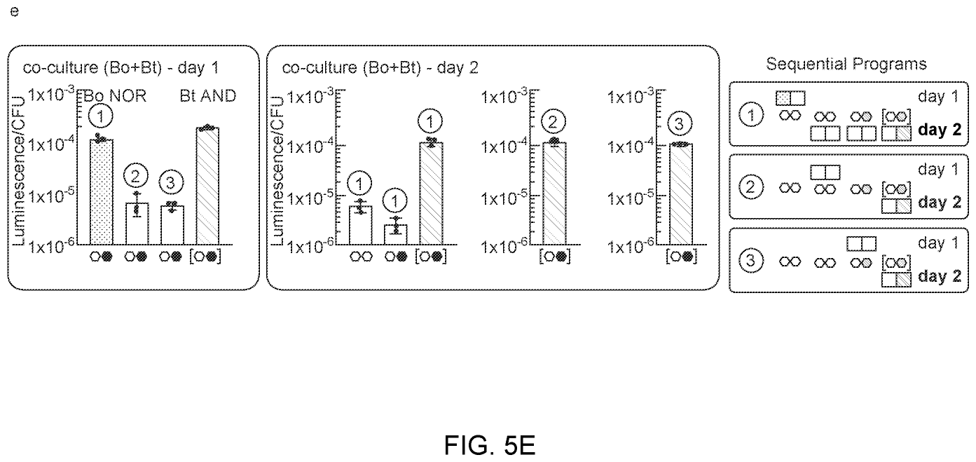

Controlling carbon utilization in Bacteroides via combinational (two-input) programming in monoculture. Given the strong performance of the single-input logical operations in managing the knockdown of a select PUL, it was contemplated that a fundamental two-input logic gates could be constructed to demonstrate more complex decision-making in the context of carbon utilization in select Bacteroides chassis cells in monoculture. First, a simple AND gate was built and tested to regulate the uptake and utilization of inulin and amylopectin in B. uniformis and B. thetaiotaomicron, respectively. In addition, in the B. ovatus chassis cell a NOR gate as well as an OR gate was built and tested in which the production of the SusC transporter was regulated implicating in the uptake of inulin in monoculture. Congruent with the loss-of-function (fitness) via inverted logic imposed by BUFFER regulated CRISPRi, the AND gates resulted in loss of fitness only when both ligands were present (FIG. 5A and FIG. 16E). In contrast, the NOR gate was permissive in the B. ovatus chassis cell when one or more ligands were present (FIG. 5B). In turn, the OR gate in the B. ovatus chassis cell only resulted in fitness of the monoculture when both ligands were absent (FIG. 5C). This set of experiments demonstrated the application of single-layer (AND, NOR) in addition to feedforward (OR) two-input logic gates as tools that can be used to manage the fitness of individual Bacteroides in monoculture.

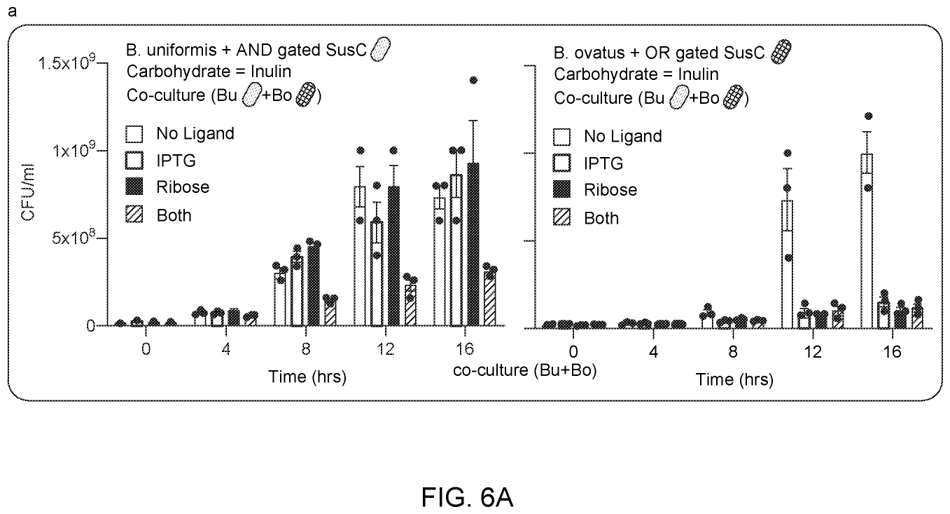

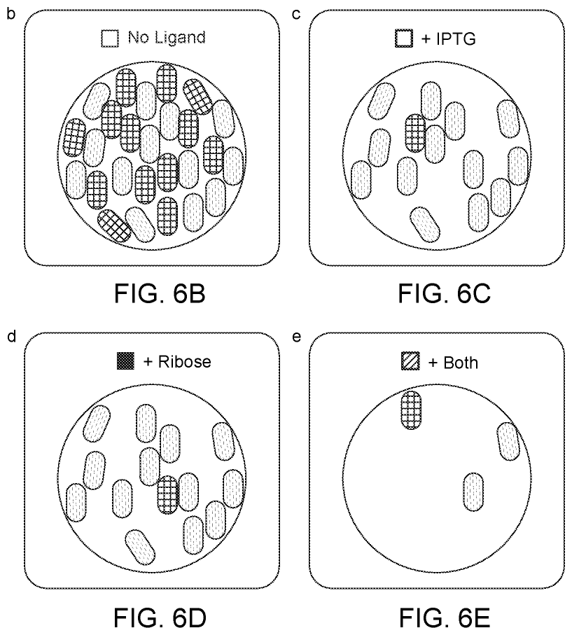

Controlling communal carbon utilization in Bacteroides via combinational programming in co-culture. Finally, a simple consortium composed of B. uniformis with an AND gate and B. ovatus with an OR gate regulating the production of sgRNAs complementary to the SusC transporters involved in inulin uptake in each chassis cell was constructed (FIG. 6). The purpose of this experiment was to demonstrate the ability to asymmetrically program the fitness of a co-culture with a defined communal carbon source. To accomplish this, a different assay was implemented in which fitness of each Bacteroides species in co-culture was accounted for via colony forming units (CFU)—given that the batch growth assay in liquid media was not amenable to distinguishing between chassis cells. Each chassis was intentionally constructed with a different antibiotic resistance to allow for their distinguishment when plated on selective BHI agar plates (Methods). The CFU assay for B. uniformis and B. ovatus in monoculture was congruent with the results obtained in batch growth—accordingly, the two assays are regarded as comparable (FIGS. 11F and FIGS. 16F and 16G). In brief, at 12 hours or greater when the co-culture had no ligands present, B. ovatus and B. uniformis retained similar fitness in co-culture (FIG. 6). Upon the addition of IPTG or D-ribose, only B. uniformis retained fitness and could uptake inulin. However, upon the addition of both ligands, B. ovatus and B. uniformis both experienced loss-of-fitness in the presence of the sole carbon source inulin, as expected.

### Discussion

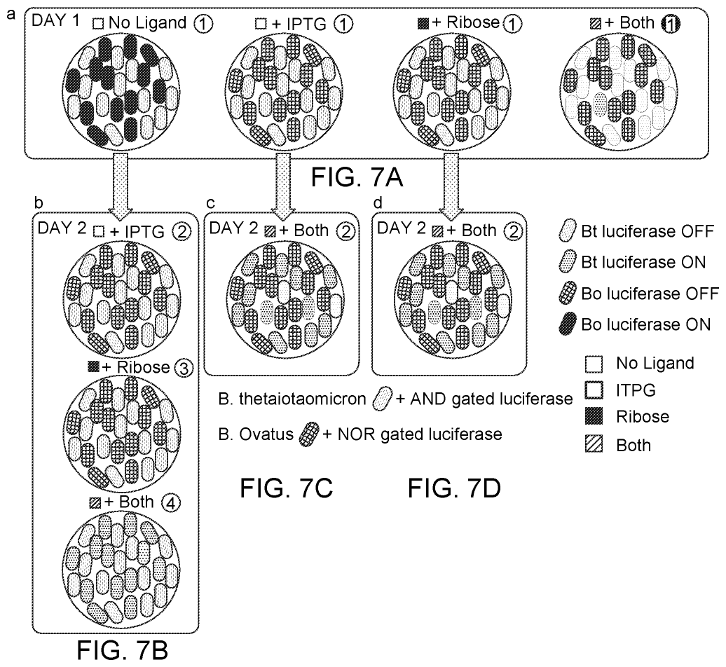

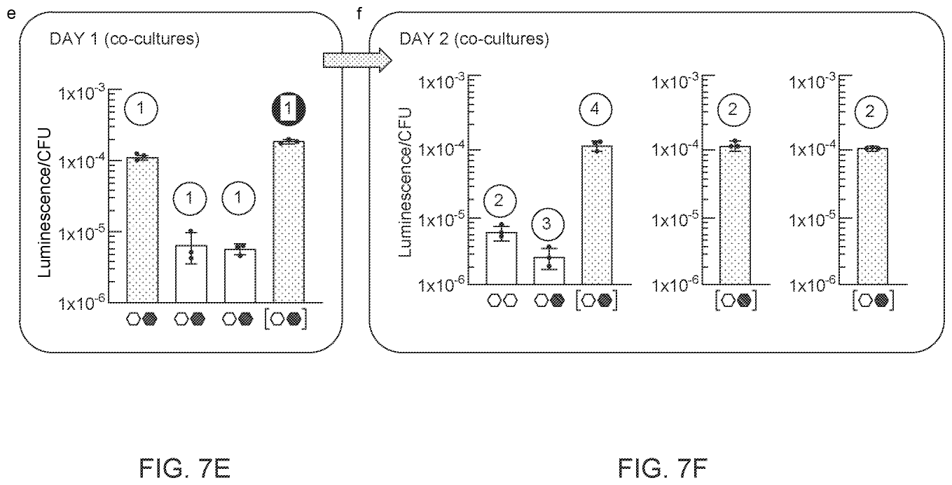

Programmable Bacteroides in co-culture. Herein, given the constraint that one out of 16 Boolean logical operations (simple transcriptional programs) can be imbued in a given Bacteroides chassis cell, the programming space for two chassis cells in co-culture can be defined by 256 non-synonymous input-output sets (FIG. 13). As presented, the input-output consortia sets can facilitate concurrent and sequential (repeated-addition) information processing for gain-of-function for a given Bacteroides co-culture. In addition, this programming structure allows the user to construct systems that can imbue symmetric and asymmetric gene regulation between two (or more) chassis cells. To demonstrate sequential programming with asymmetric gene regulation ability, a simple consortium composed of B. thetaiotaomicron and B. ovatus imbued with an AND logical operation and a NOR logical operation was constructed, respectively. Each logical operation regulated the production of luciferase as a proxy for gain-of-function in a given chassis cell (FIG. 7). Similar to gain-of-function transcriptional programming, a loss-of-function between chassis cells can be programmed, asymmetrically, concurrently, and sequentially, also see Example 2.

Consortium transcriptional programming offers a powerful tool that can be used for the advanced study of the gut microbiota. Transcriptional programming can be regarded as universal, and it is contemplated that other consortia (beyond the human gut) with complex decision-making capabilities can be imbued. In addition to the ability to use this platform to study community behavior, the programmable Bacteroides communities can also be used as the foundation for the development of living therapeutics—which will be the focus of future studies. Notably, the simple sugars allolactose (the natural analog of IPTG) and D-ribose can be consumed and show no evidence of toxicity to Bacteroides or host (human) primary cells—in support of progressing this technology to an advanced living therapeutic.

### Methods

Bacterial strains and media. Bacteroides strains used in this study were B. thetaiotaomicron (ATCC 29148), B. fragilis (ATCC 25285), B. ovatus (ATCC 8483), B. uniformis (ATCC 8492), and B. vulgatus (ATCC 8482). Bacteroides strains were routinely cultured anaerobically at 37° C. without shaking using TYG broth or BHI agar (Difco), unless otherwise specified. One liter of TYG broth contains: [10 g tryptone, 5 g yeast extract, 2.5 g D-glucose, 0.5 g L-cysteine, 13.6 g KH₂PO₄, 9.2 mg MgSO₄, 1 g NaHCO₃, 80 mg NaCl, 8 mg CaCl₂, 1 mg menadione, 0.218 mg FeSO₄, 5 μg vitamin B12, and 1 ml histidine hematin solution (1.2 mg/ml hematin in 0.2 M histidine, pH 8.0)]. L-cysteine was resuspended in water and sterile filtered (0.2 μm VWR 28145-477). Menadione was resuspended in 100% ethanol. L-cysteine and menadione were prepared and added to autoclaved media immediately prior to inoculation. Antibiotics for Bacteroides were used as appropriate: erythromycin (25 μg/ml), gentamicin (200 μg/ml), and tetracycline (2 μg/ml). IPTG and D-ribose were used as inducers at a final concentration of 10 mM, unless otherwise specified. E. coli strains used were EC100D pir-116 (for cloning) and S17-1λ pir (for conjugation). E. coli harboring pNBU-based plasmids were routinely cultured aerobically in LB Miller Media at 37° C. with shaking, or on LB agar, supplemented with 100 μg/ml carbenicillin.

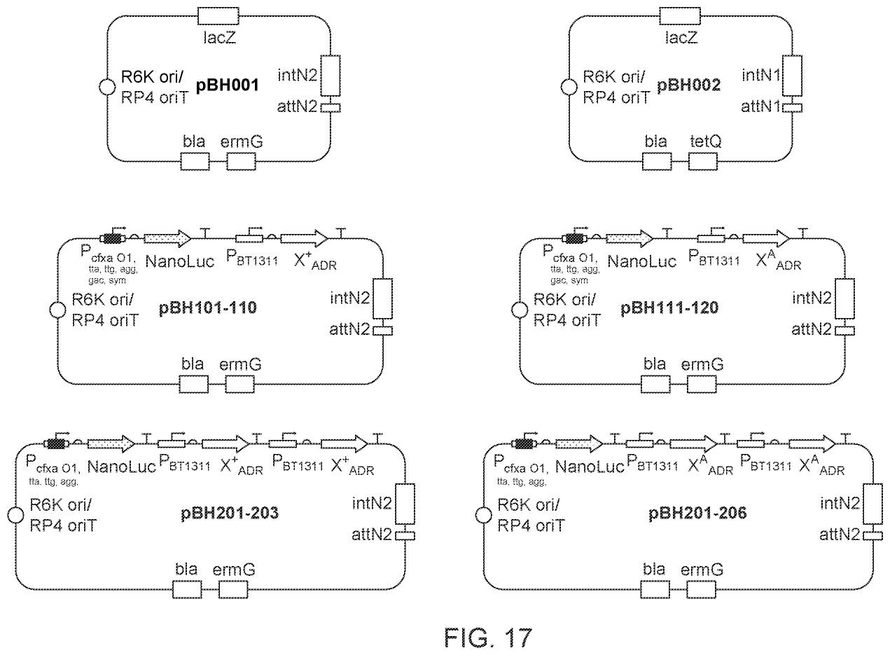

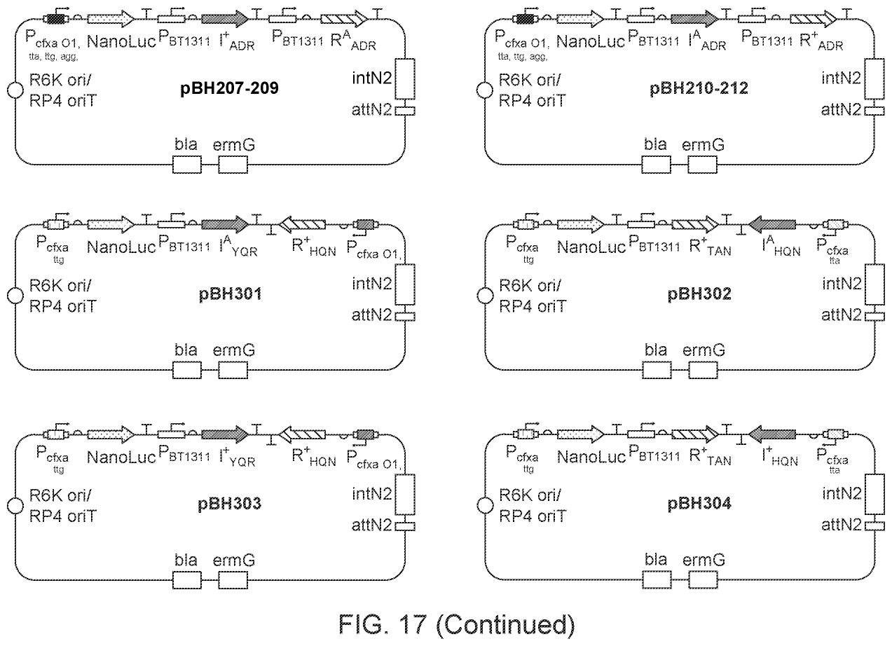

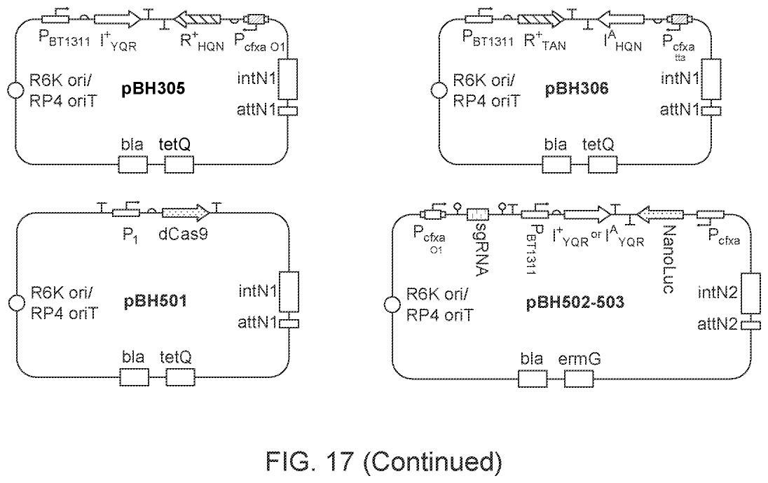

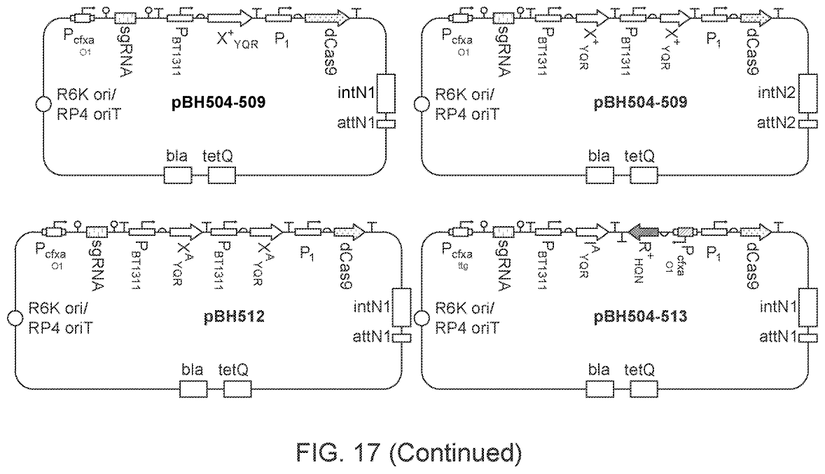

Cloning and plasmid construction. The backbone vectors for pNBU1 and pNBU2 were kind gifts from C. Voigt (MIT). Transcription factors were cloned from in-house vectors while NanoLuc was provided on the pNBU2 vector from C. Voigt. All molecular cloning was performed in E. coli EC100D pir-116. Genetic constructs were created using Golden Gate assembly and Gibson cloning. DNA modules were subcloned into a pUC-based vector for ease of manipulation before performing final assemblies. Q5 polymerase (NEB M0491L) was used for PCR involved in cloning while Phusion polymerase (NEB M0532L) was used for colony PCR. T4 DNA ligase (NEB M0202L) and BsmBI-v2 (R0739L) were used for Golden Gate cloning. NEBuilder HiFi DNA Assembly Master Mix (NEB E2621X) was used for Gibson cloning. All DNA primers were synthesized by Eurofins Genomics. The DNA sequences of all constructs were verified by Sanger sequencing (Eurofins Genomics). Plasmids were visualized using ApE software. Relevant plasmid maps are given in FIG. 17.

Conjugation of Bacteroides. E. coli S17-1λ pir was used for conjugation of plasmids into Bacteroides. The pNBU1 vector harbors intN1 which mediates site-specific recombination of the attN1 site of pNBU1 and the attB1 site located at the 3′ end of a tRNA-Leu gene in Bacteroides genomes. Similarly, the pNBU2 vector harbors intN2 which mediates site-specific recombination of the attN2 site of pNBU2 and one of two attB2 sites located at the 3′ ends of tRNA-Ser genes in Bacteroides genomes. Simultaneous insertion of pNBU2 vectors at both sites was never observed, likely due to the necessity of having at least one functional tRNA-Ser gene. Thus, only single copy genetic circuits were stably delivered into Bacteroides genomes. Donor cultures of E. coli S17-1λ pir transformed with the appropriate pNBU1 or pNBU2 construct and recipient cultures of Bacteroides were separately grown to OD600 ˜0.5. For all strains except B. fragilis, 1 ml of donor culture and 1 ml of recipient culture were pelleted by centrifugation (5000×g 5 min.) separately and resuspended in 1 ml of PBS. This step was then repeated for a second wash. The cultures were then mixed at a ratio of 1:10 (donor:receiver) and pelleted again by centrifugation. Cells were resuspended in 100 μl PBS and spot plated on a BHI agar plate. The mating lawn was grown aerobically at 37° C. for >16 hours before being scraped into 3 ml of PBS. Serial dilutions were plated on BHI agar supplemented with gentamicin and either erythromycin for pNBU2 constructs or tetracycline for pNBU1 constructs. Resultant colonies were picked into TYG after 24-48 hours of anaerobic growth. Site-specific integration was confirmed using genome-specific primers. B. fragilis conjugation efficiency was significantly lower for unknown reasons. To remedy this, 2 ml of donor culture and 2 ml of recipient culture were combined 1:1 after the PBS wash steps. The remainder of the conjugation procedure was performed as described above.

Luciferase assay. All luciferase assays were performed using TYG broth. Overnight TYG cultures of Bacteroides were diluted 1:100 into 200 μl fresh media in a conical bottom polystyrene 96-well microplate (Nunc 249952) with the appropriate combinations of inducers. The culture was incubated statically in a Mitsubishi rectangular jar equipped with anaerobic gas packs (Mitsubishi R685070) for ˜12-14 hours to achieve a final OD of ˜0.5-0.8. 100 μl of culture was then transferred to a black, clear-bottom 96-well microplate (Corning 3631) to measure OD600. The remaining 100 μl culture was pelleted by centrifugation (4000×g 10 min.) after which the supernatant was removed. The pellet was resuspended in 20 μl of Bugbuster Mastermix (Millipore 71456) and incubated at room temperature for 30 minutes to facilitate cell lysis. The Promega Nano-Glo assay kit was used to determine expression of NanoLuc. Assay buffer and substrate were mixed as per the manufacturer recommendation (1:50 ratio of substrate to buffer). 10 μl of this mixture was transferred to a well of a flat-bottom white 96-well microplate (Costar 3912) containing 80 μl DI water. Following cell lysis, 10 μl of lysate was added to the microplate well and mixed by pipetting. After 5 minutes of incubation, the luminescence was measured with a Spectramax M2e plate reader (Molecular Devices) with 800v gain and 30 reads per well. Data was collected with SoftMax Pro Software. Background luminescence generated from Bugbuster with no cells was subtracted from each sample. Luminescence was then normalized to colony forming units (CFU) based on standard curves relating OD600 to CFU (due to the presence of heme in the growth media, OD600 measurements follow non-linear patterns when compared to CFU). For the CRISPRi luciferase knockdown experiments only, the precultures were grown in the presence and absence of inducer before being seeded into TYG with the same inducer conditions. All other precultures for luciferase assays were grown without inducer. Data was analyzed using Microsoft Excel and Graphpad Prism.

Orthogonality of DNA-binding domains and operators. To determine non-cognate interactions between DNA-binding domains (DBD) and operators, all combinations of DBDs and operators were tested for each transcription factor, yielding a set of 80 “off-diagonal” combinations (in addition to the 20 cognate interactions). To facilitate testing these interactions, 5 reporter strains of B. thetaiotaomicron were created by integrating a pNBU2 plasmid containing the NanoLuc reporter gene fused to 1 of the 5 promoter/operator pairs. These reporter strains were then integrated with pNBU1 vectors containing each of the 16 transcription factors containing the DBD not associated with their specific NanoLuc operator. The expression of NanoLuc with and without inducer was measured as described above.

CRISPRi growth curves and minimal media co-culture. Long term anaerobic culture was performed in an anaerobic chamber (Whitley, DG250) with an atmosphere of 10% H₂, 10% CO₂, and 80% N₂ (Airgas X03NI80C2000511). Bacteroides strains harboring CRISPRi circuits were first grown overnight in TYG broth (no inducer). The following morning these cultures were diluted 1:100 into fresh TYG with and without inducer(s) and grown until mid-log phase (˜6 hours). At this point the cultures were diluted 1:200 into defined minimal media (MM) containing the same inducer(s) present in the precultures. One liter of MM contains: [1.12 g (NH₄)₂SO₄, 1 g NaHCO₃, 13.6 g KH₂PO₄, 0.88 g NaCl, 5.55 mg CaCl₂), 9.5 mg MgCl₂, 1 mg menadione, 0.218 mg FeSO₄, 5 μg vitamin B12, 0.5 g L-cysteine, 1 ml histidine hematin solution, and 5 g of defined carbohydrate source]. 10 mg/ml (2X) stocks of amylopectin and inulin were autoclaved and immediately mixed with the MM components (sterile filtered) before being placed in the anaerobic chamber. TYG and MM were pre-reduced in the anaerobic chamber for >24 hours before being inoculated. IPTG was added to a final concentration of 10 mM and D-ribose was added to a final concentration of 1 mM when used as inducers. For continuous OD600 measurements, the final MM cultures were prepared in 200 μl volumes in black, clear-bottom 96-well plates. These plates were grown at 37° C. inside a portable spectrophotometer (Cerillo Stratus) placed inside the anaerobic chamber. OD600 was recorded every 20 minutes to generate growth curves. For co-culture experiments, separate precultures were grown for each species as described above. For these experiments, four precultures of each species were grown in parallel, each containing a different combination of IPTG and D-ribose (no inducer, IPTG only, D-ribose only, and both inducers). Prior to MM inoculation, the OD600 of each preculture was measured. B. uniformis and B. ovatus were then seeded together into four separate 2 ml MM cultures (containing the four combinations of inducers), with the appropriate precultures being used to seed each MM culture as described above. Based on the preculture OD600 measurements, each species was seeded at an initial density of OD600 ˜0.005. The MM co-cultures were gently mixed with pipetting, and a 10 μl aliquot was removed to assess initial population density. Additional 10 μl aliquots were removed every 4 hours for 16 hours. At the time of removal, each 10 μl aliquot was 10-fold serially diluted in sterile PBS over 7 orders of magnitude. 5 μl of each dilution was spot plated in triplicate on separate BHI agar plates supplemented with erythromycin (to assess B. uniformis growth) or tetracycline (to assess B. ovatus growth). After 24 hours of anaerobic growth, colonies were counted for each time point and species to generate separate growth curves.

Sequential programming in co-culture. B. thetaiotaomicron and B. ovatus were precultured separately in TYG with no inducers for 8 hours. After measuring the OD600 of each culture, fresh 1 ml cultures containing all combinations of inducers were seeded with both strains such that the initial OD600 of each species was ˜0.005. These four cultures were grown for 12 hours and then assayed for luciferase activity (Methods). At this time, the inducer-free culture was diluted 1:100 into three separate 1 ml cultures containing either IPTG, D-ribose, or both ligands. The IPTG-containing and D-ribose-containing cultures were similarly diluted 1:100 into new 1 ml cultures containing both ligands. These five new cultures were grown for 12 hours and subsequently assayed for luciferase activity.

### Data Availability

The sequences of the following plasmids are provided in GenBank and as Source Data with respective accession numbers: pBH001-pBH002 (ON060706-ON060707), pBH101-pBH120 (ON060708-ON060727), pBH201-pBH212 (ON060728-ON060739), pBH301-pBH306 (ON060740-ON060745), pBH501-pBH513 (ON060746-ON060758).

### Example 2: Transcriptional Programming in a Bacteroides consortium

Intelligent biotic system—definition. Herein, an intelligent biotic system is defined as one or more chassis cells capable of (i) decision-making, (ii) coupled memory development, (iii) and communication between chassis cells and/or the host.

Low performing transcription factors in B. thetaiotaomicron—Justification and alternate design. Most of the transcription factors displayed inadequate fold-changes when regulating promoters with an operator at the core or proximal position alone. Generally, weak repression was observed evidenced by high basal expression levels when the transcription factor was bound to DNA. It was contemplated that the performance of a given logical operation could be improved via increasing the apparent affinity of the transcription factor by doubling the number of DNA binding sites by way of tandem operators. The general design of the in-tandem operator-promoter was composed of two DNA operators, one intercalated between the −33 and −7 hexamer and the other proximal to the TSS (FIGS. 8 and 9). In principle, this design maintains the ability to concurrently direct the binding of one or more cognate transcription factors while preserving orthogonal DNA binding. NOTE: The general form of this genetic architecture has been designated as a series-parallel (SE-PA) operator function.

Transcriptional programming and construction of feedforward gates. The development of a complete set of 16 logical operations via transcriptional programming is predicated on a definitive bottom-up combinational rule set. Specifically, single-input single-output operations (BUFFER and NOT) represent the fundamental binaries, that can be systematically combined to create all proper two input single-output operations (AND, NOR, A NIMPLY B, B NIMPLY A, OR, NAND, A IMPLY B, B IMPLY A, XOR, and XNOR). Rational construction of feedforward gates was informed by the performances of the individual transcription factors (FIG. 9). The PO1 and Ptta promoters were chosen for the first layer due to their higher maximum output when compared to the other inducible promoters developed. This was done to ensure a saturating amount of transcription factor would be produced to act on the final output promoter. The Pttg promoter was chosen as the final output promoter given its high performance (minimal “leaky” expression) when controlled by any of the four XHQN transcription factors (X=I+, IA, R+ or RA).

Circuit compression and factors beyond the inducible promoters. Circuit compression is defined as a reduction in the number of inducible promoters between any two genetic circuits with comparable operation or function. It should be noted that other factor such as the number of constitutive promoters that are required to operate the circuit are equivalent (or fewer) between said genetic circuits. The Cello circuits discussed herein are constructed via in inversion3 which will utilize equivalent numbers of constitutive promoters relative to transcriptional programming, or by way of the concurrent expression of dCas94, which will utilize an additional constitutive promoter relative to transcriptional programming (T-Pro). Accordingly, given that the number of constitutive promoters used in transcriptional programming will be equal or less than synonymous Cello circuits, the number of constitutive promoters was not factored in to the accounting for compression—although in such cases where this becomes significant constitutive promoters can be included.

It was contemplated that given two synonymous circuits (e.g., XOR—Cello vs. XOR—T-Pro, FIG. 3G) the compressed circuit in which promoter strengths and RBS strengths on average (i.e., translation and transcription) are on par between inducible promoters, the compressed circuit will utilize fewer cellular resources. To test this assertion would require an assessment of growth rates, ribosome profiling, and RNAseq analysis—in addition to using approximately equivalent production machinery, i.e., promoter strength and RBS strength, and perhaps normalizing protein lifetimes—which is beyond the scope of the present invention.

Cello Gates. Cello circuit design3 leverages tandem promoters to create OR and NOR gates that can be connected in a modular fashion. The OR gate was developed by placing two distinct, inducible promoters upstream of a sequence of interest such that induction of either or both promoters resulted in production of the downstream target5 (FIG. 15G). The NOR gate was achieved by inverting the OR gate and adding a second regulated output promoter (FIG. 15D).

Specifically, the output of the OR gate is a repressor that acts on a second regulated promoter, controlling production of the final output. The tandem promoter setup allows for the construction of a 2-promoter OR gate rather than a 4-promoter OR gate which was achieved using a pure layering approach (FIG. 18G). Additionally, the resulting NOR gate uses the same number of promoters as required from a layering approach (FIG. 15D). The use of tandem promoters results in several technical challenges—evident from the general architecture. Namely, unequal output levels may be observed for an OR gate if the tandem promoters affect one another's activity. Nielsen and coworkers describe one such phenomenon as “roadblocking”, where a downstream promoter prevents the upstream promoter from transcribing the target sequence. Accordingly, roadblocking limits the number of regulated promoters that can be used in a tandem fashion, adding an additional constraint to Cello designed circuits.

Dynamics of repeated addition and concentration dependence. As demonstrated herein, biological signal processing can be achieved by way of allosteric transcription factors (native and engineered). For example, in regulatory systems that utilize the lactose repressor, an input signal results in the induction of the transcription factor and objectively switches gene expression from an OFF-state to an ON-state. In the given biological system to revert the gene expression back to the OFF-state requires the aggressive dilution of the input signal which can take one or more days to achieve in a typical biotic system. Kinetic studies using the engineered BANDPASS and BANDSTOP transcription factors have shown that collection of signal processing filters can switch between states of gene expression within a few minutes (opposed to days). It is contemplated that given that I⁺_{YQR}, R⁺_{YQR}, I^{A}_{YQR}, and R^{A}_{YQR} are predicated on the same topology and basic functional mechanism of the repeated addition programs have similar dynamic features. In addition, the maintenance of an induced ON-state or OFF-state will require ligand concentrations of −1 mM or higher. Noting that said features will be important in subsequent implementation of this methodology. Given that the collection of transcription factors are only inducible at higher ligand concentrations than would be observed in native environments, the unintended activation of said genetic circuits is mitigated, see FIG. 11. In addition, transcriptional programs that involve repeated addition are thought to also have the capacity to rapidly transition between states based on observations of the dynamics of systems with similar mechanistic features.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1
Plasmid Constructs and the corresponding sequence identification numbers.

| | Genetic | | | Sequence |
|---|---|---|---|---|
| Designation | Parts | Backbone | Description | ID No. |
| pBH001 | intN2_ErmG_Bla_LacZ_BsmBIsites | NBU2 | cloning | SEQ ID |
| | | | vector | NO: 29 |
| pBH002 | intN1_TetQ_Bla_LacZ_BsmBIsites | NBU1 | cloning | SEQ ID |
| | | | vector | NO: 30 |
| pBH101 | LacI(YQR)_pCFXA_O1CP_nanoluc | NBU2 | BUFFER Gate | SEQ ID |
| | | | LacI(ADR) | NO: 31 |
| pBH102 | LacI(TAN)_pCFXA_OttaCP_nanoluc | NBU2 | BUFFER Gate | SEQ ID |
| | | | LacI(ADR) | NO: 32 |
| pBH103 | LacI(KSL)_pCFXA_OaggCP_nanoluc | NBU2 | BUFFER Gate | SEQ ID |
| | | | LacI(ADR) | NO: 33 |
| pBH104 | LacI(HQN)_pCFXA_OttgCP_nanoluc | NBU2 | BUFFER Gate | SEQ ID |
| | | | LacI(ADR) | NO: 34 |
| pBH105 | LacI(GKR)_pCFXA_OgacCP_nanoluc | NBU2 | BUFFER Gate | SEQ ID |
| | | | LacI(ADR) | NO: 35 |
| pBH106 | RbsR(YQR)_pCFXA_O1CP_nanoluc | NBU2 | BUFFER Gate | SEQ ID |
| | | | LacI(ADR) | NO: 36 |
| pBH107 | RbsR(TAN)_pCFXA_OttaCP_nanoluc | NBU2 | BUFFER Gate | SEQ ID |
| | | | LacI(ADR) | NO: 37 |
| pBH108 | RbsR(KSL)_pCFXA_OaggCP_nanoluc | NBU2 | BUFFER Gate | SEQ ID |
| | | | LacI(ADR) | NO: 38 |
| pBH109 | RbsR(HQN)_pCFXA_OttgCP_nanoluc | NBU2 | BUFFER Gate | SEQ ID |
| | | | LacI(ADR) | NO: 39 |
| pBH110 | RbsR(GKR)_pCFXA_OgacCP_nanoluc | NBU2 | BUFFER Gate | SEQ ID |
| | | | LacI(ADR) | NO: 40 |
| pBH111 | IA(9)(YQR)_pCFXA_O1CP_nanoluc | NBU2 | NOT Gate | SEQ ID |
| | | | IA(9)(ADR) | NO: 41 |
| pBH112 | IA(9)(TAN)_pCFXA_OttaCP_nanoluc | NBU2 | NOT Gate | SEQ ID |
| | | | IA(9)(ADR) | NO: 42 |
| pBH113 | IA(9)(KSL)_pCFXA_OaggCP_nanoluc | NBU2 | NOT Gate | SEQ ID |
| | | | IA(9)(ADR) | NO: 43 |
| pBH114 | IA(9)(HQN)_pCFXA_OttgCP_nanoluc | NBU2 | NOT Gate | SEQ ID |
| | | | IA(9)(ADR) | NO: 44 |
| pBH115 | IA(9)(GKR)_pCFXA_OgacCP_nanoluc | NBU2 | NOT Gate | SEQ ID |
| | | | IA(9)(ADR) | NO: 45 |
| pBH116 | RA(1)(YQR)_pCFXA_O1CP_nanoluc | NBU2 | NOT Gate | SEQ ID |
| | | | RA(1)(ADR) | NO: 46 |
| pBH117 | RA(1)(TAN)_pCFXA_OttaCP_nanoluc | NBU2 | NOT Gate | SEQ ID |
| | | | RA(1)(ADR) | NO: 47 |
| pBH118 | RA(1)(KSL)_pCFXA_OaggCP_nanoluc | NBU2 | NOT Gate | SEQ ID |
| | | | RA(1)(ADR) | NO: 48 |
| pBH119 | RA(1)(HQN)_pCFXA_OttgCP_nanoluc | NBU2 | NOT Gate | SEQ ID |
| | | | RA(1)(ADR) | NO: 49 |
| pBH120 | RA(1)(GKR)_pCFXA_OgacCP_nanoluc | NBU2 | NOT Gate | SEQ ID |
| | | | RA(1)(ADR) | NO: 50 |
| pBH201 | LacI(YQR)_RbsR(YQR)_pCFXA_O1CP_nanoluc | NBU2 | AND Gate | SEQ ID |
| | | | | NO: 51 |
| pBH202 | LacI(TAN)_RbsR(TAN)_pCFXA_OttaCP_nanoluc | NBU2 | AND Gate | SEQ ID |
| | | | | NO: 52 |
| pBH203 | LacI(KSL)_RbsR(KSL)_pCFXA_OaggCP_nanoluc | NBU2 | AND Gate | SEQ ID |
| | | | | NO: 53 |
| pBH204 | IA(9)(YQR)_RA(1)(YQR)_pCFXA_O1CP_nanoluc | NBU2 | NOR Gate | SEQ ID |
| | | | | NO: 54 |
| pBH205 | IA(9)(TAN)_RA(1)(TAN)_pCFXA_OttaCP_nanoluc | NBU2 | NOR Gate | SEQ ID |
| | | | | NO: 55 |
| pBH206 | IA(9)(KSL)_RA(1)(KSL)_pCFXA_OaggCP_nanoluc | NBU2 | NOR Gate | SEQ ID |
| | | | | NO: 56 |
| pBH207 | LacI(YQR)_RA(1)(YQR)_pCFXA_O1CP_nanoluc | NBU2 | I NIMPLY R | SEQ ID |
| | | | Gate | NO: 57 |
| pBH208 | LacI(TAN)_RA(1)(TAN)_pCFXA_OttaCP_nanoluc | NBU2 | I NIMPLY R | SEQ ID |
| | | | Gate | NO: 58 |
| pBH209 | LacI(KSL)_RA(1)(KSL)_pCFXA_OaggCP_nanoluc | NBU2 | I NIMPLY R | SEQ ID |
| | | | Gate | NO: 59 |
| pBH210 | IA(9)(YQR)_RbsR(YQR)_pCFXA_O1CP_nanoluc | NBU2 | R NIMPLY I | SEQ ID |
| | | | Gate | NO: 60 |
| pBH211 | IA(9)(TAN)_RbsR(TAN)_pCFXA_OttaCP_nanoluc | NBU2 | R NIMPLY I | SEQ ID |
| | | | Gate | NO: 61 |
| pBH212 | IA(9)(KSL)_RbsR(KSL)_pCFXA_OaggCP_nanoluc | NBU2 | R NIMPLY I | SEQ ID |
| | | | Gate | NO: 62 |
| pBH301 | IA(9)(YQR)_pCFXA_O1CP_RbsR(HQN)_pCFXA_OttgCP_nanoluc | NBU2 | OR Gate | SEQ ID |
| | | | | NO: 63 |
| pBH302 | RbsR(TAN)_pCFXA_ttaCP_IA(9)(HQN)_pCFXA_OttgCP_nanoluc | NBU2 | NAND Gate | SEQ ID |
| | | | | NO: 64 |
| pBH303 | LacI(YQR)_pCFXA_O1CP_RbsR(HQN)_pCFXA_OttgCP_nanoluc | NBU2 | I IMPLY R | SEQ ID |
| | | | Gate | NO: 65 |
| pBH304 | RbsR(TAN)_pCFXA_ttaCP_LacI(HQN)_pCFXA_OttgCP_nanoluc | NBU2 | R IMPLY I | SEQ ID |
| | | | Gate, partial | NO: 66 |
| | | | XNOR gate | |
| pBH305 | LacI(YQR)_pCFXA_O1CP_RbsR(HQN) | NBU1 | XNOR Gate | SEQ ID |
| | | | (partial) | NO: 67 |
| pBH306 | RbsR(TAN)_pCFXA_ttaCP_IA(9)(HQN) | NBU1 | XOR Gate | SEQ ID |
| | | | (partial) | NO: 68 |
| pBH401 | pCFXA_O1_nanoluc | NBU2 | Reporter gene | — |
| | | | for off- | |
| | | | diagonal | |
| | | | testing | |
| pBH402 | pCFXA_Otta_nanoluc | NBU2 | Reporter gene | — |
| | | | for off- | |
| | | | diagonal | |
| | | | testing | |
| pBH403 | pCFXA_Oagg_nanoluc | NBU2 | Reporter gene | — |
| | | | for off- | |
| | | | diagonal | |
| | | | testing | |
| pBH404 | pCFXA_Ottg_nanoluc | NBU2 | Reporter gene | — |
| | | | for off- | |
| | | | diagonal | |
| | | | testing | |
| pBH405 | pCFXA_Ogac_nanoluc | NBU2 | Reporter gene | — |
| | | | for off- | |
| | | | diagonal | |
| | | | testing | |
| pBH406 | LacI(YQR) | NBU1 | Single TF for | — |
| | | | off-diagonal | |
| | | | testing | |
| pBH407 | LacI(TAN) | NBU1 | Single TF for | — |
| | | | off-diagonal | |
| | | | testing | |
| pBH408 | LacI(KSL) | NBU1 | Single TF for | — |
| | | | off-diagonal | |
| | | | testing | |
| pBH409 | LacI(HQN) | NBU1 | Single TF for | — |
| | | | off-diagonal | |
| | | | testing | |
| pBH410 | LacI(GKR) | NBU1 | Single TF for | — |
| | | | off-diagonal | |
| | | | testing | |
| pBH411 | IA(9)(YQR) | NBU1 | Single TF for | — |
| | | | off-diagonal | |
| | | | testing | |
| pBH412 | IA(9)(TAN) | NBU1 | Single TF for | — |
| | | | off-diagonal | |
| | | | testing | |
| pBH413 | IA(9)(KSL) | NBU1 | Single TF for | — |
| | | | off-diagonal | |
| | | | testing | |
| pBH414 | IA(9)(HQN) | NBU1 | Single TF for | — |
| | | | off-diagonal | |
| | | | testing | |
| pBH415 | IA(9)(GKR) | NBU1 | Single TF for | — |
| | | | off-diagonal | |
| | | | testing | |
| pBH416 | RbsR(YQR) | NBU1 | Single TF for | — |
| | | | off-diagonal | |
| | | | testing | |
| pBH417 | RbsR(TAN) | NBU1 | Single TF for | — |
| | | | off-diagonal | |
| | | | testing | |
| pBH418 | RbsR(KSL) | NBU1 | Single TF for | — |
| | | | off-diagonal | |
| | | | testing | |
| pBH419 | RbsR(HQN) | NBU1 | Single TF for | — |
| | | | off-diagonal | |
| | | | testing | |
| pBH420 | RbsR(GKR) | NBU1 | Single TF for | — |
| | | | off-diagonal | |
| | | | testing | |
| pBH421 | RA(1)(YQR) | NBU1 | Single TF for | — |
| | | | off-diagonal | |
| | | | testing | |
| pBH422 | RA(1)(TAN) | NBU1 | Single TF for | — |
| | | | off-diagonal | |
| | | | testing | |
| pBH423 | RA(1)(KSL) | NBU1 | Single TF for | — |
| | | | off-diagonal | |
| | | | testing | |
| pBH424 | RA(1)(HQN) | NBU1 | Single TF for | — |
| | | | off-diagonal | |
| | | | testing | |
| pBH425 | RA(1)(GKR) | NBU1 | Single TF for | — |
| | | | off-diagonal | |
| | | | testing | |
| pBH501 | p1_dCas9 | NBU1 | Constitutive | SEQ ID |
| | | | dCas9 | NO: 69 |
| pBH502 | LacI(YQR)_pCFXA_O1CP_nano4sgRNA_pCFXA_nanoluc | NBU2 | LacI- | SEQ ID |
| | | | controlled | NO: 70 |
| | | | sgRNA for | |
| | | | nanoluc | |
| | | | knockdown | |
| pBH503 | IA(9)(YQR)_pCFXA_O1CP_nano4sgRNA_pCFXA_nanoluc | NBU2 | IA(9)- | SEQ ID |
| | | | controlled | NO: 71 |
| | | | sgRNA for | |
| | | | nanoluc | |
| | | | knockdown | |
| pBH504 | LacI(YQR)_pCFXA_O1CP_AmyC3sgRNA_p1_dCas9 | NBU1 | LacI- | SEQ ID |
| | | | controlled | NO: 72 |
| | | | sgRNA | |
| | | | for Bt | |
| | | | Amylopectin | |
| | | | susC | |
| | | | knockdown | |
| pBH505 | RbsR(YQR)_pCFXA_O1CP_AmyC3sgRNA_p1_dCas9 | NBU1 | RbsR- | SEQ ID |
| | | | controlled | NO: 73 |
| | | | sgRNA | |
| | | | for Bt | |
| | | | Amylopectin | |
| | | | susC | |
| | | | knockdown | |
| pBH506 | LacI(YQR)_pCFXA_O1CP_InuC4sgRNA_p1_dCas9 | NBU1 | LacI- | SEQ ID |
| | | | controlled | NO: 74 |
| | | | sgRNA for | |
| | | | Bo Inulin | |
| | | | susC | |
| | | | knockdown | |
| pBH507 | RbsR(YQR)_pCFXA_O1CP_InuC4sgRNA_p1_dCas9 | NBU1 | RbsR- | SEQ ID |
| | | | controlled | NO: 75 |
| | | | sgRNA for | |
| | | | Bo Inulin | |
| | | | susC | |
| | | | knockdown | |
| pBH508 | LacI(YQR)_pCFXA_O1CP_InuC6sgRNA_p1_dCas9 | NBU1 | LacI- | SEQ ID |
| | | | controlled | NO: 76 |
| | | | sgRNA | |
| | | | for Bu Inulin | |
| | | | susC | |
| | | | knockdown | |
| pBH509 | RbsR(YQR)_pCFXA_O1CP_InuC6sgRNA_p1_dCas9 | NBU1 | RbsR- | SEQ ID |
| | | | controlled | NO: 77 |
| | | | sgRNA | |
| | | | for Bu Inulin | |
| | | | susC | |
| | | | knockdown | |
| pBH510 | LacI(YQR)_RbsR(YQR)_pCFXA_O1CP_AmyC3sgRNA_p1_dCas9 | NBU2 | AND gate for | SEQ ID |
| | | | Bt | NO: 78 |
| | | | Amylopectin | |
| | | | susC | |
| | | | knockdown | |
| pBH511 | LacI(YQR)_RbsR(YQR)_pCFXA_O1CP_InuC6sgRNA_p1_dCas9 | NBU2 | AND gate for | SEQ ID |
| | | | Bu Inulin | NO: 79 |
| | | | susC | |
| | | | knockdown | |
| pBH512 | IA(9)(YQR)_RA(1)(YQR)_pCFXA_O1CP_InuC4sgRNA_p1_dCas9 | NBU1 | NOR gate for | SEQ ID |
| | | | Bo Inulin | NO: 80 |
| | | | susC | |
| | | | knockdown | |
| pBH513 | IA(9)(YQR)_pCFXA_O1CP_RbsR(HQN)_pCFXA_OttgCP_InuC4sgRNA_p1_dCas9 | NBU1 | OR gate for Bo | SEQ ID |
| | | | Inulin susC | NO: 81 |
| | | | knockdown | |

TABLE 2
Sequences of genetic components

| Name | Type | Sequence |
|---|---|---|
| LacI(YQR) | gene | gtgaaaccagtaacgttatacgatgtcgcagagtatgccggtgtctcttatcagaccgtttc |
| | | ccgcgtggtgaaccaggccagccacgtttctgcgaaaacgcgggaaaaagtggaagcg |
| | | gcgatggcggagctgaattacattcccaaccgcgtggcacaacaactggcgggcaaac |
| | | agtcgttgctgattggcgttgccacctccagtctggccctgcacgcgccgtcgcaaattgt |
| | | cgcggcgattaaatctcgcgccgatcaactgggtgccagcgtggtggtgtcgatggtaga |
| | | acgaagcggcgtcgaagcctgtaaaacggcggtgcacaatcttctcgcgcaacgcgtca |
| | | gtgggctgatcattaactatccgctggatgaccaggatgccattgctgtggaagctgcctg |
| | | cactaatgttccggcgttatttcttgatgtctctgaccagacacccatcaacagtattattttct |
| | | cccatgaagacggtacgcgactgggcgtggagcatctggtcgcattgggtcaccagcaa |
| | | atcgcgctgttagcgggcccattaagttctgtctcggcgcgtctgcgtctggctggctggc |
| | | ataaatatctcactcgcaatcaaattcagccgatagcggaacgggaaggcgactggagtg |
| | | ccatgtccggttttcaacaaaccatgcaaatgctgaatgagggcatcgttcccactgcgat |
| | | gctggttgccaacgatcagatggcgctgggcgcaatgcgcgccattaccgagtccgggc |
| | | tgcgcgttggtgcggatatctcggtagtgggatacgacgataccgaagacagctcatgtta |
| | | tatcccgccgttaaccaccatcaaacaggattttcgcctgctggggcaaaccagcgtgga |
| | | ccgcttgctgcaactctctcagggccaggcggtgaagggcaatcagctgttgcccgtctc |
| | | actggtgaaaagaaaaaccaccctggcgcccaatacgcaaaccgcctctccccgcgcgt |
| | | tggccgattcattaatgcagctggcacgacaggtttcccgactggaaagcgggcagtga |
| | | (SEQ ID NO: 1) |
| RbsR(YQR) | gene | gtgaaaccagtaacgttatacgatgtcgcagagtatgccggtgtctcttatcagaccgtttc |
| | | ccgcgtggtgaaccaggccagccacgtttctgcgaaaacgcgggaaaaagtggaagcg |
| | | gcgatggcggagctcaattacattcccaaccgcgtggcacaacaactggcgggcaaag |
| | | cgtcgcataccattggcatgttgatcactgccagtaccaatcctttctattcagaactggtgc |
| | | gtggcgttgaacgcagctgcttcgaacgcggttatagtctcgtcctttgcaataccgaagg |
| | | cgatgaacagcggatgaatcgcaatctggaaacgctgatgcaaaaacgcgttgatggctt |
| | | gctgttactgtgcaccgaaacgcatcaaccttcgcgtgaaatcatgcaacgttatccgaca |
| | | gtgcctactgtgatgatggactgggctccgttcgatggcgacagcgatcttattcaggata |
| | | actcgttgctgggcggagacttagcaacgcaatatctgatcgataaaggtcatacccgtat |
| | | cgcctgtattaccggcccgctggataaaactccggcgcgcctgcggttggaaggttatcg |
| | | ggcggcgatgaaacgtgcgggtctcaacattcctgatggctatgaagtcactggtgatttt |
| | | gaatttaacggcgggtttgacgctatgcgccaactgctatcacatccgctgcgtcctcagg |
| | | ccgtctttaccggaaatgacgctatggctgttggcgtttaccaggcgttatatcaggcagag |
| | | ttacaggttccgcaggatatcgcggtgattggctatgacgatatcgaactggcaagctttat |
| | | gacgccaccattaaccactatccaccaaccgaaagatgaactgggggagctggcgattg |
| | | atgtactcatccatcggataacccagccgacccttcagcaacaacgattacaacttactcc |
| | | gattctgatggaacgcggttcggcttagctggtgaaaagaaaaaccaccctggcgccca |
| | | atacgcaaaccgcctctccccgcgcgttggccgattcattaatgcagctggcacgacagg |
| | | tttcccgactggaaagcgggcagtga (SEQ ID NO: 2) |
| IA(9)(YQR) | gene | gtgaaaccagtaacgttatacgatgtcgcagagtatgccggtgtctcttatcagaccgtttc |
| | | ccgcgtggtgaaccaggccagccacgtttctgcgaaaacgcgggaaaaagtggaagcg |
| | | gcgatggcggagctgaattacattcccaaccgcgtggcacaacaactggcgggcaaac |
| | | agtcgttgctgattggcgttgccacctccagtctggccctgcacgcgctgtcgcaaattgtc |
| | | gcggcgattaaatctcgcgcctatcaactgggtgccagcgtgttcgtgtcgatggtagaac |
| | | gaagcggcatcgaagcctgtaaaacggcggtgcacaatcttctcgcgcaacgcgtcagt |
| | | gggctgatcattaactatccgctggataaccaggatgccattgctgtggaagctgcctgca |
| | | ctaatgttccggcgttatttcttgatgtctctgaccagacacccatcaacagtattattttctcc |
| | | catgaagacggtacgcgactgggcgtggagcatctggtcgcattgggtcaccagcaaat |
| | | cgcgctgttagcgggcccattaagttctgtctcggcgcgtctgcgtctggctggctggcat |
| | | aaatatctcactcgcaatcaaattcagccgatagcggaacgggaaggcgactggagtgc |
| | | catgtccggttttcaacaaaccatgcaaatgctgaatgagggcatcgttcccactgcgatg |
| | | ctggttgccaacgatcagatggcgctgggcgcaatgcgcgccattaccgagaccgggct |
| | | gcgcgttggtgcggatatctcggtagtgggatacgacgataccgaagacagctcatgttat |
| | | atcccgccgttaaccaccatcaaacaggattttcgcctgctggggcaaaccagcgtggac |
| | | cgcttgctgcaactctctcagggccaggcggtgaagggcaatcagctgttgcccgtctca |
| | | ctggtgaaaagaaaaaccaccctggcgcccaatacgcaaaccgcctctccccgcgcgtt |
| | | ggccgattcattaatgcagctggcacgacaggtttcccgactggaaagcgggcagtga |
| | | (SEQ ID NO: 3) |
| RA(1)(YQR) | gene | gtgaaaccagtaacgttatacgatgtcgcagagtatgccggtgtctcttatcagaccgtttc |
| | | ccgcgtggtgaaccaggccagccacgtttctgcgaaaacgcgggaaaaagtggaagcg |
| | | gcgatggcggagctcaattacattcccaaccgcgtggcacaacaactggcgggcaaag |
| | | cgtcgcataccattggcatgttgatcactgccagtaccaatcctttctattcagaactggtgc |
| | | gtggcgttgaacgcagctgcttcgaacgcggttatagtctcgatctttgcaataccgaagg |
| | | cgatgaacagcggatgaatcgcaatctggaaacgctgatgcaaaaacgcgttgatggctt |
| | | gctgttactgtgcaccgaaacgcatcaaccttcgcgtgaaatcatgcaacgttatccgaca |
| | | gtgcctactgtgatgatggactgggctccgttcgatggcgacagcgatcttattcaggata |
| | | actcgttgctgggcggagacttagcaacgcaatatctgatcgataaaggtcatacccgtat |
| | | cgcctgtattaccggcccgctggataaaactccggcgcgcctgcggttggaaggttatcg |
| | | ggcggcgatgaaacgtgcgggtctcaacattcctgatggctatgaagtcactggtgatttt |
| | | gaatttaacggcgggtttgacgctatgcgccaactgctatcacatccgctgcgtcctcagg |
| | | ccgtctttaccggaaatgacgctatggctgttggcgtttaccaggcgttatatcaggcagag |
| | | ttacaggttccgcaggatatcgcggtgattggctatgacgatatcgaactggcaagctttat |
| | | gacgccaccattaaccactatccaccaaccgaaagatgaactgggggagctggcgattg |
| | | atgtactcatccatcggataacccagccgacccttcagcaacaacgattacaacttactcc |
| | | gattctgatggaacgcggttcggcttagctggtgaaaagaaaaaccaccctggcgccca |
| | | atacgcaaaccgcctctccccgcgcgttggccgattcattaatgcagctggcacgacagg |
| | | tttcccgactggaaagcgggcagtga (SEQ ID NO: 4) |
| NanoLuc | gene | atggtttttactctggaagattttgttggcgattggcgtcagaccgcgggttataatttggatc |
| | | aagtcctggaacagggggcgtaagctctctgttccagaacctgggtgtgagcgtgacgc |
| | | cgattcagcgcatcgttctgtccggcgagaacggtctgaaaattgatattcatgtgatcatc |
| | | ccgtacgaaggcctgagcggtgaccaaatgggtcaaatcgagaaaatctttaaagtcgtc |
| | | tacccagttgacgatcaccacttcaaggttatcttgcattacggtacgctggtgattgatggt |
| | | gtgaccccgaatatgattgactatttcggccgtccgtatgaaggcattgccgtttttgacggt |
| | | aaaaagatcaccgtcaccggtaccctgtggaatggcaataagattattgacgagcgtctg |
| | | attaacccggacggcagcctgctgttccgcgtgaccatcaacggtgtcacgggttggcgt |
| | | ctgtgcgagcgcatcctggcataa (SEQ ID NO: 5) |
| dCas9 | gene | atggataagaaatactcaataggcttagctatcggcacaaatagcgtcggatgggcggtg |
| | | atcactgatgaatataaggttccgtctaaaaagttcaaggttctgggaaatacagaccgcc |
| | | acagtatcaaaaaaaatcttataggggctcttttatttgacagtggagagacagcggaagc |
| | | gactcgtctcaaacggacagctcgtagaaggtatacacgtcggaagaatcgtatttgttatc |
| | | tacaggagattttttcaaatgagatggcgaaagtagatgatagtttctttcatcgacttgaag |
| | | agtcttttttggtggaagaagacaagaagcatgaacgtcatcctatttttggaaatatagtag |
| | | atgaagttgcttatcatgagaaatatccaactatctatcatctgcgaaaaaaattggtagattc |
| | | tactgataaagcggatttgcgcttaatctatttggccttagcgcatatgattaagtttcgtggtc |
| | | attttttgattgagggagatttaaatcctgataatagtgatgtggacaaactatttatccagttg |
| | | gtacaaacctacaatcaattatttgaagaaaaccctattaacgcaagtggagtagatgctaa |
| | | agcgattctttctgcacgattgagtaaatcaagacgattagaaaatctcattgctcagctccc |
| | | cggtgagaagaaaaatggcttatttgggaatctcattgctttgtcattgggtttgacccctaat |
| | | tttaaatcaaattttgatttggcagaagatgctaaattacagctttcaaaagatacttacgatg |
| | | atgatttagataatttattggcgcaaattggagatcaatatgctgatttgtttttggcagctaag |
| | | aatttatcagatgctattttactttcagatatcctaagagtaaatactgaaataactaaggctcc |
| | | cctatcagcttcaatgattaaacgctacgatgaacatcatcaagacttgactcttttaaaagct |
| | | ttagttcgacaacaacttccagaaaagtataaagaaatcttttttgatcaatcaaaaaacgga |
| | | tatgcaggttatattgatgggggagctagccaagaagaattttataaatttatcaaaccaattt |
| | | tagaaaaaatggatggtactgaggaattattggtgaaactaaatcgtgaagatttgctgcgc |
| | | aagcaacggacctttgacaacggctctattccccatcaaattcacttgggtgagctgcatg |
| | | ctattttgagaagacaagaagacttttatccatttttaaaagacaatcgtgagaagattgaaa |
| | | aaatcttgacttttcgaattccttattatgttggtccattggcgcgtggcaatagtcgttttgcat |
| | | ggatgactcggaagtctgaagaaacaattaccccatggaattttgaagaagttgtcgataa |
| | | aggtgcttcagctcaatcatttattgaacgcatgacaaactttgataaaaatcttccaaatga |
| | | aaaagtactaccaaaacatagtttgctttatgagtattttacggtttataacgaattgacaaag |
| | | gtcaaatatgttactgaaggaatgcgaaaaccagcatttctttcaggtgaacagaagaaag |
| | | ccattgttgatttactcttcaaaacaaatcgaaaagtaaccgttaagcaattaaaagaagatt |
| | | atttcaaaaaaatagaatgttttgatagtgttgaaatttcaggagttgaagatagatttaatgct |
| | | tcattaggtacctaccatgatttgctaaaaattattaaagataaagattttttggataatgaaga |
| | | aaatgaagatatcttagaggatattgttttaacattgaccttatttgaagatagggagatgatt |
| | | gaggaaagacttaaaacatatgctcacctctttgatgataaggtgatgaaacagcttaaac |
| | | gtcgccgttatactggttggggacgtttgtctcgaaaattgattaatggtattagggataagc |
| | | aatctggcaaaacaatattagattttttgaaatcagatggttttgccaatcgcaattttatgcag |
| | | ctgatccatgatgatagtttgacatttaaagaagacattcaaaaagcacaagtgtctggaca |
| | | aggcgatagtttacatgaacatattgcaaatttagctggtagccctgctattaaaaaaggtat |
| | | tttacagactgtaaaagttgttgatgaattggtcaaagtaatggggggcataagccagaa |
| | | aatatcgttattgaaatggcacgtgaaaatcagacaactcaaaagggccagaaaaattcg |
| | | cgagagcgtatgaaacgaatcgaagaaggtatcaaagaattaggaagtcagattcttaaa |
| | | gagcatcctgttgaaaatactcaattgcaaaatgaaaagctctatctctattatctccaaaatg |
| | | gaagagacatgtatgtggaccaagaattagatattaatcgtttaagtgattatgatgtcgatg |
| | | ccattgttccacaaagtttccttaaagacgattcaatagacaataaggtcttaacgcgttctg |
| | | ataaaaatcgtggtaaatcggataacgttccaagtgaagaagtagtcaaaaagatgaaaa |
| | | actattggagacaacttctaaacgccaagttaatcactcaacgtaagtttgataatttaacga |
| | | aagctgaacgtggtggtttgagtgaacttgataaagctggttttatcaaacgccaattggttg |
| | | aaactcgccaaatcactaagcatgtggcacaaattttggatagtcgcatgaatactaaatac |
| | | gatgaaaatgataaacttattcgagaggttaaagtgattaccttaaaatctaaattagtttctg |
| | | acttccgaaaagatttccaattctataaagtacgtgagattaacaattaccatcatgcccatg |
| | | atgcgtatctaaatgccgtcgttggaactgctttgattaagaaatatccaaaacttgaatcgg |
| | | agtttgtctatggtgattataaagtttatgatgttcgtaaaatgattgctaagtctgagcaagaa |
| | | ataggcaaagcaaccgcaaaatatttcttttactctaatatcatgaacttcttcaaaacagaa |
| | | attacacttgcaaatggagagattcgcaaacgccctctaatcgaaactaatggggaaactg |
| | | gagaaattgtctgggataaagggcgagattttgccacagtgcgcaaagtattgtccatgcc |
| | | ccaagtcaatattgtcaagaaaacagaagtacagacaggcgaattctccaaggagtcaat |
| | | tttaccaaaaagaaattcggacaagcttattgctcgtaaaaaagactgggatccaaaaaaa |
| | | tatggtggttttgatagtccaacggtagcttattcagtcctagtggttgctaaggtggaaaaa |
| | | gggaaatcgaagaagttaaaatccgttaaagagttactagggatcacaattatggaaaga |
| | | agttcctttgaaaaaaatccgattgactttttagaagctaaaggatataaggaagttaaaaaa |
| | | gacttaatcattaaactacctaaatatagtctttttgagttagaaaacggtcgtaaacggatgc |
| | | tggctagtgccggagaattacaaaaaggaaatgagctggctctgccaagcaaatatgtga |
| | | attttttatatttagctagtcattatgaaaagttgaagggtagtccagaagataacgaacaaa |
| | | aacaattgtttgtggagcagcataagcattatttagatgagattattgagcaaatcagtgaatt |
| | | ttctaagcgtgttattttagcagatgccaatttagataaagttcttagtgcatataacaaacata |
| | | gagacaaaccaatacgtgaacaagcagaaaatattattcatttatttacgttgacgaatcttg |
| | | gagctcccgctgcttttaaatattttgatacaacaattgatcgtaaacgatatacgtctacaaa |
| | | agaagttttagatgccactcttatccatcaatccatcactggtctttatgaaacacgcattgatt |
| | | tgagtcagctaggaggtgactga (SEQ ID NO: 6) |
| PcfxA | promoter | atacaaagaaaattcgacaaactgttatttttctatctatttatttgggtgggaaactttagttat |
| | | gtacctttgtcggc (SEQ ID NO: 7) |
| P1 | promoter + | gataaagtttggaagataaagctaaaagttcttatctttgcagtccgaaataaagacatataa |
| | RBS | aagaaaagacacc (SEQ ID NO: 8) |
| PBT1311 | promoter + | tgatctggaagaagcaatgaaagctgctgttaagtctccgaatcaggtattgttcctgacag |
| | RBS | gtgtattcccatccggtaaacgcggatactttgcagttgatctgactcaggaataaattataa |
| | | attaaggtaagaagattgtaggataagctaatgaaatagaaaaaggatgccgtcacacaa |
| | | cttgtcggcattcttttttgttttattagttgaaaatatagtgaaaaagttgcctaaatatgtatgt |
| | | taacaaattatttgtcgtaactttgcactccaaatctgtttttaacatatggcacta (SEQ ID |
| | | NO: 9) |
| Pcfxa(O1) | promoter | tacaaagaaaattcgacaaactgttatttttctatctatttatttgaattgtgagcggataacaat |
| | | tacctttgtcggcaattgtgagcggataacaatt (SEQ ID NO: 10) |
| Pcfxa(Otta) | promoter | tacaaagaaaattcgacaaactgttatttttctatctatttatttgaattttaagcgcttaaaattta |
| | | cctttgtcggcaattttaagcgcttaaaatt (SEQ ID NO: 11) |
| Pcfxa(Ogac) | promoter | tacaaagaaaattcgacaaactgttatttttctatctatttatttgaattgacagcgctgtcaattt |
| | | acctttgtcggcaattgacagcgctgtcaatt (SEQ ID NO: 12) |
| Pcfxa(Ottg) | promoter | tacaaagaaaattcgacaaactgttatttttctatctatttatttgaattttgagcgctcaaaattt |
| | | acctttgtcggcaattttgagcgctcaaaatt (SEQ ID NO: 13) |
| Pcfxa(Oagg) | promoter | tacaaagaaaattcgacaaactgttatttttctatctatttatttgaattaggagcgctcctaattt |
| | | acctttgtcggcaattaggagcgctcctaatt (SEQ ID NO: 14) |
| Pcfxa(Osym) | promoter | tacaaagaaaattcgacaaactgttatttttctatctatttatttgaattgtgagcgctcacaattt |
| | | acctttgtcggcaattgtgagcgctcacaatt (SEQ ID NO: 15) |
| nano4 | sgRNA | gacagaacgatgcgctgaatgttttagagctagaaatagcaagttaaaataaggctagtcc |
| | | gttatcaacttgaaaaagtggcaccgagtcggtgctttttt (SEQ ID NO: 16) |
| BOinu4 | sgRNA | cctgacatcacattaccagtgttttagagctagaaatagcaagttaaaataaggctagtccg |
| | | ttatcaacttgaaaaagtggcaccgagtcggtgctttttt (SEQ ID NO: 17) |
| BUinu6 | sgRNA | gtcaactcaatgcgtaaagtgttttagagctagaaatagcaagttaaaataaggctagtccg |
| | | ttatcaacttgaaaaagtggcaccgagtcggtgctttttt (SEQ ID NO: 18) |
| BTamy3 | sgRNA | acaacattggcaccgataacgttttagagctagaaatagcaagttaaaataaggctagtcc |
| | | gttatcaacttgaaaaagtggcaccgagtcggtgctttttt (SEQ ID NO: 19) |
| HH | 5′ ribozyme | nnnnnnctgatgagtccgtgaggacgaaacgagtaagctcgtc |
| ribozyme | | (SEQ ID NO: 20) |
| ribozyme | 3′ ribozyme | ggccggcatggtcccagcctcctcgctggcgccggctgggcaacatgcttcggcatggc |
| | | gaatgggac (SEQ ID NO: 21) |
| nanoTerm | terminator | gcactctaatcgttatcggagtgcttttagattactaatcaaattgcttcta |
| | | (SEQ ID NO: 22) |
| L3S2P55 | terminator | ctcggtaccaaagacgaaCaataagacgctgaaaagcgtcttttttcgttttggtcc |
| | | (SEQ ID NO: 23) |
| L3S2P21 | terminator | ctcggtaccaaattccagaaaagaggcctcccgaaaggggggccttttttcgttttggtcc |
| | | (SEQ ID NO: 24) |
| TAN DBD | partial gene | gtgaaaccagtaacgttatacgatgtcgcagagtatgccggtgtctctaccgcgaccgttt |
| | | ccaac (SEQ ID NO: 25) |
| GKR DBD | partial gene | gtgaaaccagtaacgttatacgatgtcgcagagtatgccggtgtctctggaaagaccgttt |
| | | cccgc (SEQ ID NO: 26) |
| HQN DBD | partial gene | gtgaaaccagtaacgttatacgatgtcgcagagtatgccggtgtctctcatcagaccgtttc |
| | | caat (SEQ ID NO: 27) |
| KSL DBD | partial gene | gtgaaaccagtaacgttatacgatgtcgcagagtatgccggtgtctctaaaagcaccgtttc |
| | | cctg (SEQ ID NO: 28) |

a. For the HH ribozyme, the first 6 bp are the reverse complement of the 6 bp directly downstream of the ribozyme.
b. The DBD sequences include the first 22 residues of the gene, ending at the “R” of the “YQR” motif.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A construct comprising a plurality of nucleic acid sequences encoding
a first group of one or more regulatory core domains;
a second group of one or more regulatory core domains;
one or more DNA binding domains, wherein the first group of the one or more regulatory core domains and the second group of the one or more regulatory core domains are each linked to one of the DNA binding domains; and
one or more DNA operator elements, wherein the one or more DNA operator elements are each specifically recognized by one of the DNA binding domains.

2. The construct of claim 1, further comprising SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or variants thereof.

3. The construct of claim 1, wherein the plurality of nucleic acid sequences comprises any combination of SEQ ID NO: 1, SEQ ID NO: 2, SEQ ID NO: 3, SEQ ID NO: 4, SEQ ID NO: 5, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or variants thereof.

4. The construct of claim 1, wherein the first group of one or more regulatory core domains comprises at least one repressor or at least one anti-repressor, or a combination thereof.

5. The construct of claim 1, wherein the second group of one or more regulatory core domains comprise at least one repressors, at least one anti-repressors, or a combination thereof.

6. The construct of claim 1, wherein the first group of one or more regulatory core domains are specifically recognized by a first agent.

7. The construct of claim 6, wherein the first agent is isopropyl-β-D-1-thiogalactopyranoside.

8. The construct of claim 1, wherein the second group of one or more regulatory core domains are specifically recognized by a second agent.

9. The construct of claim 8, wherein the second agent is D-ribose.

10. The construct of claim 1, wherein the first and second group of the one or more regulatory core domains are linked to a same DNA binding domain.

11. The construct of claim 1, wherein the first and second group of the one or more regulatory core domains are linked to different DNA binding domains.

12. The construct of claim 1, wherein the construct comprises a plurality of nucleic acid sequences encoding
a first group of two regulatory core domains;
a second group of two regulatory core domains;
three DNA binding domains, wherein the first group of the regulatory core domains and second group of the regulatory core domains are each linked to one of the three DNA binding domains; and
three DNA operator elements that are each specifically recognized by one of the three DNA binding domains.

13. The construct of claim 1, further comprising a nucleic acid sequence encoding a reporter.

14. The construct of claim 1, further comprising a nucleic acid sequence encoding a dead Cas9 endonuclease (dCas9) and a single guide RNA (sgRNA).

15. The construct of claim 1, wherein the nucleic acid sequence encodes any combination of SEQ ID NO: 6, SEQ ID NO: 16, SEQ ID NO: 17, SEQ ID NO: 18, SEQ ID NO: 19, or variants thereof.

16. The construct of claim 1, further comprising SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, or variants thereof.

17. A cell comprising a construct comprising a plurality of nucleic acid sequences encoding
a first group of one or more regulatory core domains;
a second group of one or more regulatory core domains;
one or more DNA binding domains, wherein the first group of the one or more regulatory core domains and the second group of the one or more regulatory core domains are each linked to one of the DNA binding domains; and
one or more DNA operator elements, wherein the one or more DNA operator elements are each specifically recognized by one of the DNA binding domains.

18. The cell of claim 17, wherein the cell is a bacterial cell.

19. The cell of claim 19, wherein the bacterial cell is a species of Bacteroides genus selected from B. thetaiotaomicron (Bt), B. fragilis (Bf), B. vulgatus (Bv), B. ovatus (Bo), or B. uniformis (Bu).

20. A method of modifying a gastrointestinal tract microbiome in a subject, comprising
administering to the subject an effective amount of a cell comprising a construct comprising a plurality of nucleic acid sequences encoding
a first group of one or more regulatory core domains;
a second group of one or more regulatory core domains;
one or more DNA binding domains, wherein the first group of the one or more regulatory core domains and the second group of the one or more regulatory core domains are each linked to one of the DNA binding domains; and
one or more DNA operator elements, wherein the one or more DNA operator elements are each specifically recognized by one of the DNA binding domains.
